(19) United States
(12) Patent Application Publication
(10) Pub. No.: US 2025/0367302 A1
(43) Pub. Date: Dec. 4, 2025

(54) SMALL MOLECULE DRUG-OLIGONUCLEOTIDE CONJUGATE AND USE THEREOF

(71) Applicants: SHANGHAI JIAO TONG UNIVERSITY, Shanghai (CN); ZHEJIANG YIMOU BIOLOGY SCIENCE AND TECHNOLOGY CO., LTD, Anji County Huzhou (CN)

(72) Inventors: Chuan ZHANG, Shanghai (CN); Ruihua ZHU, Shanghai (CN); Yangyang HUANG, Shanghai (CN)

(21) Appl. No.: 18/877,987

(22) PCT Filed: Jun. 21, 2023
(86) PCT No.: PCT/CN2023/101555

§ 371 Date: Dec. 20, 2024

(30) Foreign Application Priority Data
Jun. 21, 2022 (CN) 202210703857.4

(51) Int. Cl. A61K 47/55; A61K 47/54; A61P 17/06; A61P 27/02; A61P 29/00

(52) U.S. Cl. CPC A61K 47/55; A61K 47/549; A61P 17/06; A61P 27/02; A61P 29/00

(57) ABSTRACT

Disclosed is a small molecule drug-oligonucleotide conjugate and use thereof, which belongs to the technical field of biomedical technology. In order to solve the problems of poor water solubility and difficulty in drug delivery of the existing small molecule immunomodulatory drug, the small molecule immunomodulatory drug is coupled with an oligonucleotide drug.

### CROSS REFERENCE TO RELATED APPLICATION

The present application is a national stage application of International Patent Application No. PCT/CN2023/101555, filed on Jun. 21, 2023, which claims priority to Chinese Patent Application No. 202210703857.4 filed to the China National Intellectual Property Administration (CNIPA) on Jun. 21, 2022 and entitled “SMALL MOLECULE DRUG-OLIGONUCLEOTIDE CONJUGATE AND USE THEREOF”, which is incorporated herein by reference in its entirety.

### REFERENCE TO SEQUENCE LISTING

A computer readable XML file entitled “Sequence Listing”, that was created on Dec. 17, 2024, with a file size of 8,3536 bytes, contains the sequence listing for this application, has been filed with this application, and is hereby incorporated by reference in its entirety.

### TECHNICAL FIELD

The present disclosure belongs to the technical field of biomedical technology, and specifically relates to a small molecule drug-oligonucleotide conjugate and use thereof.

### BACKGROUND

In recent years, there has been an explosive growth in the study on mechanisms of inflammatory response and the relationship between inflammation and disease. A large number of studies have enabled people to have a deeper understanding of the mechanisms of inflammatory response and how inflammation affects the occurrence and development of many diseases. Based on the research results obtained, it is now believed that inflammation may be a major driving factor in various diseases such as psoriasis, Sjögren's syndrome, xerophthalmia, uveitis, keratitis, conjunctivitis, atopic dermatitis, rheumatoid arthritis, inflammatory enteritis, Crohn's disease, heart disease, diabetes, cancer, asthma, inflammatory bowel disease, and Alzheimer's disease. In the common inflammatory response process, histamine molecules released by ruptured or activated mast cells, circulating basophils, and platelets, together with vascular cell adhesion molecule-1 (VCAM-1), endothelial cell adhesion molecule-1 (E-selectin), and intercellular adhesion molecule-1 (ICAM-1) in the blood circulation, collectively cause vasodilation, resulting in a significant increase in vascular permeability (Cardiovasc Hematol Disord Drug Targets 2008, 8, 252-260). The increase in vascular permeability may lead to local edema, allowing substances such as macrophages, complement system proteins, arachidic acid, kinins, cytokines, and platelet-activating factors to enter the blood vessels ([J]Allergy Clin Immunol 2010, 125, S3-S23), thereby promoting the occurrence and development of inflammation. Among these, cytokines, such as tumor necrosis factor, interleukins, lymphokines, monokines, interferons, colony-stimulating factors, as well as transforming growth factors produced by monocytes, T cells, platelets, and endothelial cells are all involved in inflammation-related responses. In addition, when components of the innate immune system, including monocytes (such as macrophages and dendritic cells) and neutrophils, are activated, inflammatory responses are enhanced.

Innate immune system recognizes bacteria and other damages through the recognition of specific ligands by transmembrane Toll-like receptors (TLRs). For example, stimulation of TLR4 on macrophages with lipopolysaccharide (LPS) triggers the synthesis of inflammatory factors such as TNF-α and IL-1β, and then stimulates the production of immunoregulatory cytokine IL-12, which is essential for adaptive immune responses. Once macrophages are activated by inflammatory factors, their lifespan increases and they produce large amounts of pro-inflammatory cytokines, including TNF-α, IL-1, IL-6, IL-12/IL-23, IL-18, and high mobility group box protein 1 (HMGB1). These cytokines will further amplify the inflammatory response and trigger the adaptive immune response. Circulating cytokines interact with specific receptors on different cell types and activate Janus kinase-signal transducer and activator of transcription (JAK-STAT), nuclear factor kappa B (NF-κB), and transforming growth factor β (TGF-β) signaling pathways, leading to inflammatory responses such as cell adhesion, increased permeability, and apoptosis, as well as increased reactive oxygen species (ROS).

The common characteristics of inflammation-related diseases are the activation of related signaling pathways and the overexpression of multiple inflammatory cytokines after the disruption of immune balance, which in turn trigger sustained inflammatory response and cause destructive effects on the body. Based on the understanding of the mechanisms of disease-induced inflammatory responses and the immune activation process, researchers are committed to developing different types of drugs for inflammation regulation with the aim of ultimately curing disease. Existing inflammation regulation drugs mainly include small molecule drugs with immunomodulatory functions, antibody drugs targeting cytokines and receptors involved in inflammatory responses, and gene drugs that regulate the expression of genes related to inflammatory signaling pathways. Commonly used small molecule drugs with immunomodulatory functions include calcineurin inhibitors, mammalian target of rapamycin (mTOR) inhibitors, glucocorticoids, and vitamin D analogs, which can be produced on a large scale at a low cost, and some small molecule drugs can also be made into oral preparations. However, the small molecule drugs generally have the disadvantages of poor water solubility, low bioavailability, and strong side effects, so they are not suitable for long-term use. Antibody drugs are usually highly specific, fast-acting, and excellent in immune regulatory effects, but they generally require injection for administration and show high production and treatment costs. In addition, long-term systemic administration can easily lead to drug resistance and other adverse reactions, with significant toxic side effects, such as reduced immunity, increased risk of infection and cancer.

Functional oligonucleotide molecules targeting inflammatory response-related genes for immune regulation (siRNA and antisense oligonucleotide (ASO)) can serve as a novel drug with low immunogenicity and a wide range of targets. Such drugs target both intracellular and extracellular targets, are not prone to drug resistance, and can regulate upstream signaling pathways, providing more possibilities for immune regulation and disease treatment. However, due to the large molecular weight and negative charge of nucleic acid drugs, it is difficult for sole nucleic acid drugs to cross the cell membrane barrier, presenting delivery difficulties. In order to achieve the effective delivery of nucleic acid drugs and exert their gene regulatory functions, it is generally necessary to introduce specific vectors to help deliver functional nucleic acids to target tissues and cells. Although viral vectors have high transfection efficiency, they still face a series of problems in actual application, such as immunogenicity, insertion mutations, and complex preparation, which hinder their clinical transformation. In addition to the viral vectors, non-viral vectors such as cationic liposomes and cationic polymers are also commonly used for the loading and delivery of nucleic acid drugs. However, cationic delivery vectors generally face high cytotoxicity and difficult quality control. Although each type of drug has its advantages, the use of a single strategy to regulate a certain target or block a certain cytokine may lead to a compensatory increase in other proinflammatory cytokines due to the complexity of inflammatory responses, making it difficult to achieve satisfactory therapeutic effects.

### SUMMARY

In view of this, a purpose of the present disclosure is to provide a small molecule drug-oligonucleotide conjugate and use thereof. In the present disclosure, the small molecule drug-oligonucleotide conjugate includes a small molecule drug that regulates immune response and a functional oligonucleotide molecule. A combination of the above two components can simultaneously act on different inflammation-related signal pathways and then produce a synergistic effect, thereby achieving a better therapeutic effect.

The present disclosure provides a small molecule drug-oligonucleotide conjugate, the small molecule drug-oligonucleotide conjugate is prepared by covalent coupling of a small molecule drug with an immunomodulatory function and a functional oligonucleotide molecule capable of regulating expression of an inflammation-related gene.

In some embodiments, the covalent coupling of the small molecule drug and the functional oligonucleotide molecule is implemented through a chemical linker.

In some embodiments, the small molecule drug acts on an immune-related signaling pathway and regulates an immune response; in some embodiments, the small molecule drug is selected from one or more of a calcineurin inhibitor, a glucocorticoid, a mammalian target of rapamycin (mTOR) inhibitor, and a vitamin D analog.

In some embodiments, the inflammation-related gene is selected from one or more of a tumor necrosis factor-a gene, an interleukin 1b gene, an interleukin 17 gene, an interleukin 23 gene, an NFKBIZ gene, an inflammasome NLRP3 gene, a JAK gene, and a PDE4 gene.

In some embodiments, the functional oligonucleotide molecule is selected from one of a double-stranded small interfering RNA (siRNA), a microRNA (miRNA), and a single-stranded antisense oligonucleotide (ASO).

In some embodiments, when the functional oligonucleotide molecule is the double-stranded siRNA or the miRNA, the small molecule drug is covalently coupled to a 3′ end of a sense strand of the functional oligonucleotide molecule; and when the functional oligonucleotide molecule is the single-stranded ASO, the small molecule drug is covalently coupled to a 3′ end or a 5′ end of the single-stranded ASO.

In some embodiments, the number of the small molecule drugs covalently coupled to each functional oligonucleotide molecule is in a range of 1 to 40.

In some embodiments, the small molecule drug is covalently coupled to a terminal of the functional oligonucleotide molecule, or the small molecule drug is covalently coupled to a side chain base or a phosphate backbone of an extended sequence at a 3′ end or a 5′ end of the functional oligonucleotide molecule;

in some embodiments, when the small molecule drug is covalently coupled to the terminal of the functional oligonucleotide molecule, the small molecule drug-oligonucleotide conjugate has a chemical structure shown in Formula 1 to Formula 14:

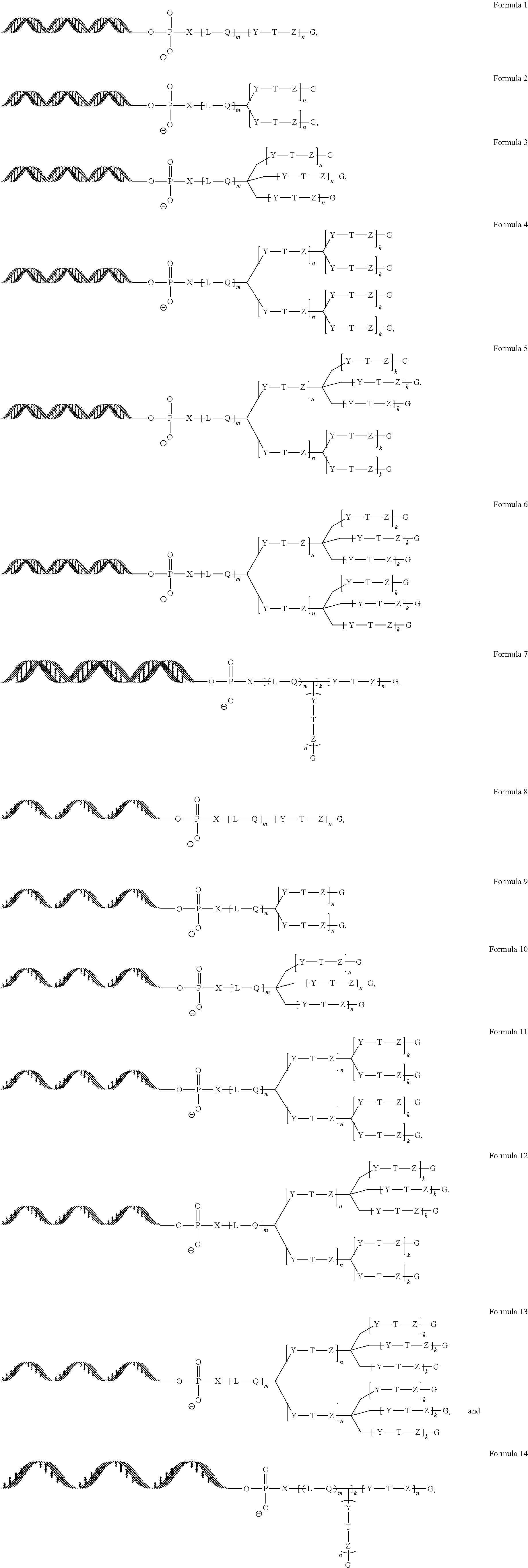

where

in Formula 1 to Formula 14, L and T are independently absent, or selected from the group consisting of —(CH₂)h- and a group formed by substituting any one or more alkylene groups in —(CH₂)h- with a group A; h is 0 to 15; the group A is selected from the group consisting of —O—, —S—, —C(O)—, —C(O)O—, —C(O)NH—, —CH(R^{C})—, —C(R′)(R″)—, —NH—, —N(R^{N})—, —S—S—, —C(R′)═C(R″)—, —C═C—,

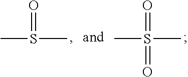

R^{C}, R^{N}, R′, and R″ in the group A represent that any one or more hydrogen atoms on a designated atom are substituted by a group B on condition that a normal valence of the designated atom is not exceeded and a stable compound is generated by substitution, and the designated atom is selected from the group consisting of a carbon atom and a nitrogen atom; the group B is selected from the group consisting of C1-C6 alkyl, C2-C6 alkenyl, C2-C6 alkynyl, cyano, hydroxyl, oxo, carboxyl, cycloalkyl, cycloalkenyl, heterocyclyl, heteroaryl, aryl, keto, alkoxycarbonyl, aryloxycarbonyl, heteroaryloxycarbonyl, and a halogen; the halogen is selected from the group consisting of F, Cl, Br, and I; and (O) in the group A represents a carbonyl oxygen atom;

in Formula 1 to Formula 14, Q, Y, and Z are independently absent, or selected from the group consisting of —O—, —S—, —C(O)—, —NH—, —CH₂—, —C(O)NH—, —NHC(O)—, —C(O)O—, —OC(O)—, —OC(O)O—, —OC(O)NH—, —NHC(O)O—, and

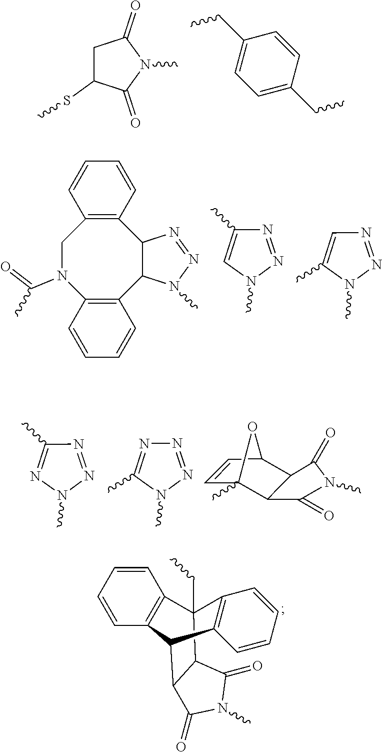

(O) in the groups Q, Y, and Z represents a carbonyl oxygen atom; [symbol] and represents a ligation site;

in Formula 1 to Formula 14, G represents a small molecule immunomodulatory drug; m, n, and k are independently 1 to 15; and X represents O or S;

in some embodiments, when the small molecule drug is covalently coupled to the side chain base or the phosphate backbone of the extended sequence at the 3′ end or the 5′ end of the functional oligonucleotide molecule, the small molecule drug-oligonucleotide conjugate has a chemical structure shown in Formula 15 to Formula 20:

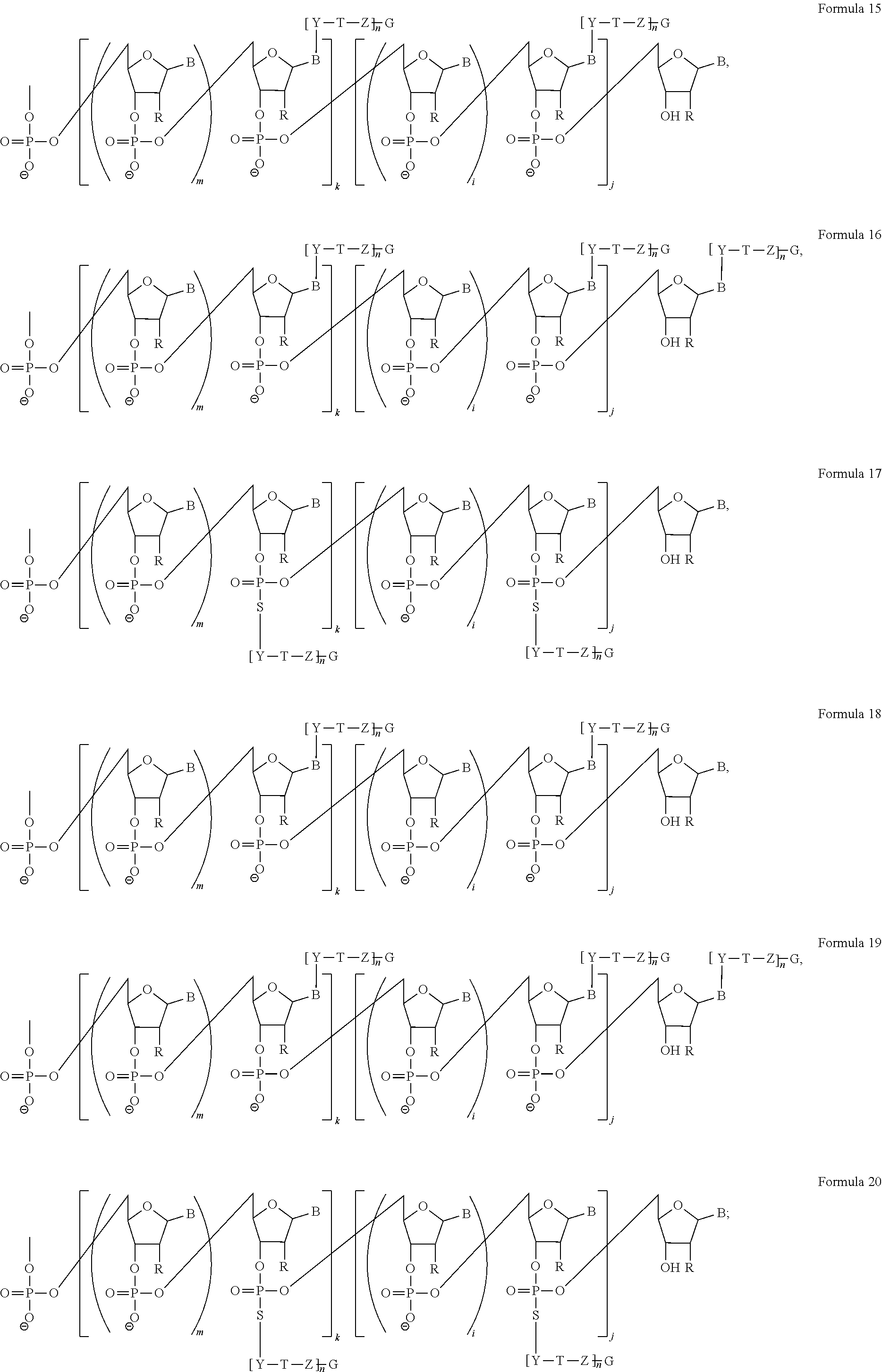

where,

in Formula 15 to Formula 20, T is absent, or selected from the group consisting of —(CH₂)h- and a group formed by substituting any one or more alkylene groups in —(CH₂)h- with a group A; h is 0 to 15; the group A is selected from the group consisting of —O—, —S—, —C(O)—, —C(O)O—, —C(O)NH—, —CH(R^{C})—, —C(R′)(R″)—, —NH—, —N(R^{N})—, —S—S—, —C(R′)═C(R″)—, —C═C—,

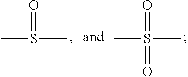

R^{C}, R^{N}, R′, and R″ in the group A represent that any one or more hydrogen atoms on a designated atom are substituted by a group B on condition that a normal valence of the designated atom is not exceeded and a stable compound is generated by substitution, and the designated atom is selected from the group consisting of a carbon atom and a nitrogen atom; the group B is selected from the group consisting of C1-C6 alkyl, C2-C6 alkenyl, C2-C6 alkynyl, cyano, hydroxyl, oxo, carboxyl, cycloalkyl, cycloalkenyl, heterocyclyl, heteroaryl, aryl, keto, alkoxycarbonyl, aryloxycarbonyl, heteroaryloxycarbonyl, and a halogen; the halogen is selected from the group consisting of F, Cl, Br, and I; and (O) in the group A represents a carbonyl oxygen atom;

in Formula 15 to Formula 20, Y and Z are independently absent, or selected from the group consisting of O, S, C(O), NH, CH₂, C(O)NH, NHC(O), C(O) O, OC(O), OC(O) O, OC(O)NH, NHC(O) O, and

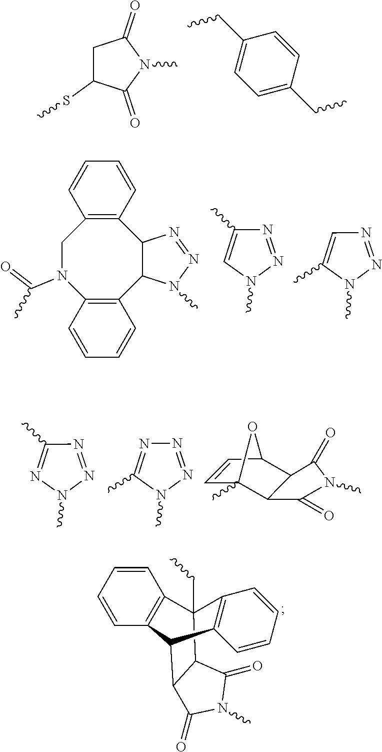

(O) in the groups Y and Z represents a carbonyl oxygen atom; and [symbol] represents a ligation site;

in Formula 15 to Formula 20, G represents an immunomodulatory inhibitor; n is 1 to 15; m and i are independently 0 to 5, and k and j are independently 1 to 20; R represents H; and B represents a nucleic acid base.

The present disclosure further provides use of the small molecule drug-oligonucleotide conjugate in preparation of a drug for treating an inflammation-related disease.

In some embodiments, the inflammation-related disease includes xerophthalmia, psoriasis, Sjögren's syndrome, uveitis, keratitis, conjunctivitis, atopic dermatitis, rheumatoid arthritis, inflammatory bowel disease, and Crohn's disease.

The present disclosure provides a small molecule drug-oligonucleotide conjugate, which is prepared by covalent coupling of a small molecule drug with an immunomodulatory function and a functional oligonucleotide molecule capable of regulating expression of an inflammation-related gene. In order to solve the problems of poor water solubility and difficulty in drug delivery of the existing small molecule immunomodulatory drug, in the present disclosure, the small molecule immunomodulatory drug is coupled with an oligonucleotide drug. An excellent hydrophilicity of the oligonucleotide drug can improve solubility properties of the small molecule drug, improve tissue distribution of the drug in vivo, and promote drug delivery and absorption. Moreover, the hydrophobic small molecule drug can in turn promote entry of the oligonucleotide drug into cells, thereby enhancing its ability to regulate target genes. Furthermore, co-delivery of the small molecule drug and the functional oligonucleotide molecule achieves coordinated regulation of different targets for an inflammatory response, thereby achieving better disease treatment effects and realizing safe and efficient medication. In addition, the two drug molecules assist each other, can realize the preparation of a novel drug delivery system without additional carriers, and can efficiently deliver the functional oligonucleotide molecule and small molecule immunomodulatory drug at the same time. This process achieves synergistically regulating inflammatory responses, thereby providing a better solution for the treatment of various inflammatory-related diseases.

### BRIEF DESCRIPTION OF THE DRAWINGS

FIG. 1 shows a schematic diagram of the preparation routes of azide-modified cyclosporin A (CsA-N₃) and monocyclosporin A-NFKBIZ ASO covalent conjugate (CsA-ASO_{NFKBIZ});

FIG. 2 shows the ¹H NMR spectrum of chloromethyl carbonate cyclosporin A (CsA-C1);

FIG. 3 shows the ¹³C NMR spectrum of chloromethyl carbonate cyclosporin A (CsA-Cl);

FIG. 4 shows the MALDI-TOF mass spectrum of chloromethyl carbonate cyclosporin A (CsA-C1);

FIG. 5 shows the ¹H NMR spectrum of azidohexanoic acid carbonate-modified cyclosporin A (CsA-N₃);

FIG. 6 shows the MALDI-TOF mass spectrum of azidohexanoic acid carbonate-modified cyclosporin A (CsA-N₃);

FIG. 7 shows the gel electrophoresis of a coupling product of CsA-N₃ and DBCO-modified ASO, where the efficient synthesis of CsA-ASO_{NFKBIZ} conjugate is confirmed by 10% denatured polyacrylamide gel electrophoresis detection;

FIGS. 8A-8B show the cellular uptake of CsA-ASO_{NFKBIZ}-h conjugate;

FIG. 9 shows the mRNA expression level of the target gene NFKBIZ detected by real-time fluorescence quantitative PCR after cells are treated with CsA-ASO_{NFKBIZ}-h and control sample;

FIGS. 10A-10C show the effect evaluation of CsA-ASO_{NFKBIZ}-m nanomicelles in treating xerophthalmia; where FIG. 10A shows the sodium fluorescein staining of mouse eyes at 0, 7, and 14 d of treatment; FIG. 10B shows the sodium fluorescein scoring data analysis of mouse eyes at 0, 7, and 14 d of treatment; and FIG. 10C shows the results of a phenol red cotton thread tear test of mice at 0, 7, and 14 d of treatment;

FIG. 11 shows the 10% denatured polyacrylamide gel electrophoresis and 1% agarose gel electrophoresis of 2CsA-ASO_{NFKBIZ} nanomicelles;

FIG. 12 shows the mRNA expression level of the target gene NFKBIZ detected by real-time fluorescence quantitative PCR after cells are treated with 2CsA-ASO_{NFKBIZ}-h and control sample;

FIGS. 13A-13C show the effect evaluation of 2CsA-ASO_{NFKBIZ}-m nanomicelles in treating xerophthalmia; where FIG. 13A shows the sodium fluorescein staining of mouse eyes at 0, 7, and 14 d of treatment; FIG. 13B shows the sodium fluorescein scoring data analysis of mouse eyes at 0, 7, and 14 d of treatment; and FIG. 13C shows the results of a phenol red cotton thread tear test of mice at 0, 7, and 14 d of treatment;

FIG. 14 shows the characterization of 3CsA-ASO_{NFKBIZ} conjugate and self-assembled nanomicelles thereof, where the left side shows the results of 10% denatured polyacrylamide gel electrophoresis, and a right side shows the results of 0.5% agarose gel electrophoresis;

FIG. 15 shows the mRNA expression level of the target gene NFKBIZ detected by real-time fluorescence quantitative PCR after cells are treated with 3CsA-ASO_{NFKBIZ}-h and control sample;

FIGS. 16A-16C show the effect evaluation of 3CsA-ASO_{NFKBIZ}-m nanomicelles in treating xerophthalmia; where FIG. 16A shows the sodium fluorescein staining of mouse eyes at 0, 7, and 14 d of treatment; FIG. 16B shows the sodium fluorescein scoring data analysis of mouse eyes at 0, 7, and 14 d of treatment; and FIG. 16C shows the results of a phenol red cotton thread tear test of mice at 0, 7, and 14 d of treatment;

FIG. 17 shows a schematic diagram of the synthesis of aminotris [(2-propynyloxy)methyl]methane;

FIG. 18 shows the ¹H NMR spectrum of Boc-aminotris [(2-propynyloxy)methyl]methane;

FIG. 19 shows a schematic diagram of the synthesis of CsA3-ASO_{NFKBIZ} conjugate;

FIG. 20 shows characterization of trialkynyl-modified ASO_{NFKBIZ} conjugate by 20% denatured polyacrylamide gel electrophoresis;

FIG. 21 shows characterization of CsA₃-ASO_{NFKBIZ} nanomicelles by 0.5% agarose gel electrophoresis;

FIGS. 22A-22B show the hydrated particle size (FIG. 22A) and morphology (FIG. 22B) of CsA₃-ASO_{NFKBIZ} nanomicelles;

FIG. 23 shows the mRNA expression level of a target gene NFKBIZ detected by real-time fluorescence quantitative PCR after cells are treated with CsA₃-ASO_{NFKBIZ-h} nanomicelles and control sample;

FIGS. 24A-24C show the effect evaluations of CsA₃-ASO_{NFKBIZ-m} nanomicelles in treating xerophthalmia; where FIG. 24A shows the sodium fluorescein staining of mouse eyes at 0 and 14 d of treatment; FIG. 24B shows the sodium fluorescein scoring data analysis of mouse eyes at 0 and 14 d of treatment; and FIG. 24C shows the results of a phenol red cotton thread tear test of mice at 0 and 14 d of treatment;

FIGS. 25A-25B show the 15% denatured polyacrylamide gel electrophoresis results of CsA-SiRNA_{NFKBIZ}, 2CSA-siRNA_{NFKBIZ}, and 3CsA-siRNA_{NFKBIZ} coupled molecules, and the 0.5% agarose gel electrophoresis results of 2CsA-siRNA_{NFKBIZ} and 3CSA-siRNA_{NFKBIZ} nanomicelles;

FIG. 26 shows expression levels of IκB-(protein in human corneal epithelial cells after being treated with CsA-siRNA_{NFKBIZ-h} conjugate, 2CsA-siRNA_{NFKBIZ-h} nanomicelles, 3CsA-siRNA_{NFKBIZ-h} nanomicelles, and control sample;

FIG. 27 shows the mRNA expression levels of NFKBIZ gene detected by real-time fluorescence quantitative PCR after treatment of human ocular epithelial cells with CsA-siRNA_{NFKBIZ}-h conjugate, 2CsA-siRNA_{NFKBIZ-h} nanomicelles, 3CsA-siRNA_{NFKBIZ-h} nanomicelles, and control sample;

FIGS. 28A-28C show the effect evaluations of CsA-siRNA_{NFKBIZ-m} conjugate, 2CsA-siRNA_{NFKBIZ-m} nanomicelles, and 3CsA-siRNA_{NFKBIZ-m} nanomicelles in treating xerophthalmia; where FIG. 28A shows the sodium fluorescein staining of mouse eyes at 0, 7, and 14 d of treatment;

FIG. 28B shows the sodium fluorescein scoring data analysis of mouse eyes at 0, 7, and 14 d of treatment; and FIG. 28C shows the results of a phenol red cotton thread tear test of mice at 0, 7, and 14 d of treatment;

FIG. 29 shows the synthesis steps of carboxyethyl bromo-CsA;

FIG. 30 shows the LC-MS spectrum of carboxyethyl bromo-CsA;

FIG. 31 shows the 10% denatured polyacrylamide gel electrophoresis and 1% agarose gel electrophoresis of 20CsA-ASO_{NFKBIZ} nanomicelles;

FIG. 32 shows the mRNA expression level of a gene NFKBIZ detected by real-time fluorescence quantitative PCR after are treated with 20CsA-ASO_{NFKBIZ-h} nanomicelles and control sample;

FIGS. 33A-33B show the effect evaluation of 20CsA-ASO_{NFKBIZ-m} nanomicelles in treating xerophthalmia; where FIG. 33A shows the sodium fluorescein scoring data analysis of mouse eyes at 0 and 14 d of treatment; and FIG. 33B shows the results of a phenol red cotton thread tear test of mice at 0 and 14 d of treatment;

FIGS. 34A-34D show the expression levels of various inflammatory mRNAs in human corneal epithelial cells treated with different target 3CsA-siRNA nanomicelles and control sample detected by real-time fluorescence quantitative PCR;

FIGS. 35A-35D show the expression levels of various inflammatory factors in human corneal epithelial cells treated with different target 3CsA-siRNA nanomicelles and control sample detected by ELISA;

FIGS. 36A-36B show the effect evaluations of 3CsA-siRNA nanomicelles in treating xerophthalmia; where FIG. 36A shows the sodium fluorescein scoring data analysis of mouse eyes at 0 and 14 d of treatment; and FIG. 36B shows the results of a phenol red cotton thread tear test of mice at 0 and 14 d of treatment;

FIGS. 37A-37C show the expression levels of various inflammatory targets in human corneal epithelial cells treated with different target 3CsA-siRNA nanomicelles and control sample detected by real-time fluorescence quantitative PCR;

FIGS. 38A-38B show the effect evaluations of 3CsA-siRNA nanomicelles in treating xerophthalmia; where FIG. 38A shows the sodium fluorescein scoring data analysis of mouse eyes at 0 and 14 d of treatment; and FIG. 38B shows the results of a phenol red cotton thread tear test of mice at 0 and 14 d of treatment;

FIGS. 39A-39D show the expression levels of various inflammatory mRNAs in human corneal epithelial cells treated with different target 3CsA-ASO nanomicelles and control sample detected by real-time fluorescence quantitative PCR;

FIGS. 40A-40D show the expression levels of various inflammatory factors in human corneal epithelial cells treated with different target of 3CsA-ASO nanomicelles and control sample detected by ELISA;

FIGS. 41A-41B show the effect evaluation of 3CsA-ASO nanomicelles in treating xerophthalmia; where FIG. 41A shows the sodium fluorescein scoring data analysis of mouse eyes at 0 and 14 d of treatment; and FIG. 41B shows the results of a phenol red cotton thread tear test of mice at 0 and 14 d of treatment;

FIGS. 42A-42C shows expression levels of various inflammatory mRNAs in human corneal epithelial cells treated with different target 20CsA-ASO nanomicelles and control sample detected by real-time fluorescence quantitative PCR;

FIGS. 43A-43B show the effect evaluation of 20CsA-ASO nanomicelles in treating xerophthalmia; where FIG. 43A shows the sodium fluorescein scoring data analysis of mouse eyes at 0 and 14 d of treatment; and FIG. 43B shows the results of a phenol red cotton thread tear test of mice at 0 and 14 d of treatment;

FIG. 44 shows a schematic diagram of the synthesis of RAP-Bz-Br;

FIGS. 45A-45B show the ¹H NMR spectrum (FIG. 45A) and ¹³C NMR spectrum (FIG. 45B) of TK-Bz-Br;

FIGS. 46A-46B show the ¹H NMR spectrum (FIG. 46A) and ¹³C NMR spectrum (FIG. 46B) of RAP-31-O-TMS-42-Bz-Br;

FIGS. 47A-47B show the successful preparation of 10RAP-ASO conjugate and 10RAP-nanomicelles characterized by 15% denatured polyacrylamide gel electrophoresis (FIG. 47A) and 1% agarose gel electrophoresis (FIG. 47B);

FIGS. 48A-48B show the hydrated particle size (FIG. 48A) and TEM morphology (FIG. 48B) of 10RAP-ASO_{NFKBIZ} nanomicelles;

FIG. 49 shows the mRNA expression level of NFKBIZ detected by real-time fluorescence quantitative PCR (RT-qPCR);

FIGS. 50A-50B show the effect evaluations of 10RAP-ASO_{NFKBIZ-m} nanomicelles in treating xerophthalmia; where FIG. 50A shows the sodium fluorescein scoring data analysis of mouse eyes at 0, 7, and 14 d of treatment; and FIG. 50B shows the results of a phenol red cotton thread tear test of mice at 0, 7, and 14 d of treatment;

FIG. 51 shows the mRNA expression levels of gene NFKBIZ in HaCaT cells after co-incubation with CsA₃-ASO_{NFKBIZ-h} nanomicelles and control sample detected by RT-PCR;

FIGS. 52A-52C show the secretions of different inflammatory factors in HaCaT cells after treatment with CsA₃-ASO_{NFKBIZ-h} nanomicelles and control sample detected by ELISA;

FIG. 53 shows a model phenotype of the mouse back on the 7th day of modeling;

FIG. 54 shows photos of skin lesions on the back of psoriasis-like model mice on days 0 and 7 after treatment with smear administration;

FIGS. 55A-55B show the changes in ear and back skin thickness of psoriasis-like model mice after smear administration;

FIGS. 56A-56D show the scores of erythema, scaling, and skin thickening at the back lesions of psoriasis-like model mice after smear administration and the psoriasis lesion area severity index score;

FIG. 57 shows a schematic diagram of the synthesis of carboethyl bromo-triamcinolone acetonide (TA-Br);

FIG. 58 shows the ¹H NMR spectrum of TA-Br;

FIG. 59 shows the ¹³C NMR spectrum of TA-Br;

FIG. 60 shows the LC-MS spectrum of TA-Br;

FIG. 61 shows the successful preparation of 10TA-ASO_{NFKBIZ} conjugate characterized by 15% denatured polyacrylamide gel electrophoresis;

FIG. 62 shows the successful preparation of 10TA-ASO_{NFKBIZ} nanomicelles characterized by 1% agarose gel electrophoresis;

FIG. 63 shows the mRNA expression levels of gene NFKBIZ in HaCaT cells after co-incubation with 10TA-ASO_{NFKBIZ-h} nanomicelles and control sample detected by RT-PCR;

FIGS. 64A-64B show the changes in ear and back skin thickness of psoriasis-like model mice after smear administration;

FIGS. 65A-65D show the scores of erythema, scaling, and skin thickening at the back lesions of psoriasis-like model mice before and after treatment and the psoriasis lesion area severity index score;

FIG. 66 shows photos of skin lesions on the back of psoriasis-like model mice on days 0 and 7 after treatment;

FIG. 67 shows a schematic diagram of the synthesis of Cal-Bz-Br;

FIG. 68 shows the ¹H NMR spectrum of DTPA-Bz-Br;

FIG. 69 shows the ¹H NMR spectrum of Cal-Bz-Br;

FIG. 70 shows the LC-MS spectrum of Cal-Bz-Br;

FIG. 71 shows the successful preparation of 10Cal-ASO_{NFKBIZ} conjugate characterized by 15% denatured polyacrylamide gel electrophoresis;

FIG. 72 shows the successful preparation of 10Cal-ASO_{NFKBIZ} nanomicelles characterized by 1% agarose gel electrophoresis;

FIGS. 73A-73B show the hydrated particle (FIG. 73A) and TEM morphology (FIG. 73B) of 10Cal-ASO_{NFKBIZ} nanomicelles;

FIG. 74 shows a critical micelle concentration of 10Cal-ASO_{NFKBIZ} nanomicelles;

FIG. 75 shows the mRNA expression levels of gene NFKBIZ in HaCaT cells after co-incubation with 10Cal-ASO_{NFKBIZ-h} nanomicelles and control sample detected by RT-PCR;

FIGS. 76A-76B show the changes in skin thickness of psoriasis-like model mice during treatment;

FIGS. 77A-77D show the scores of erythema, scaling, and skin thickening at the back lesions of psoriasis-like model mice before and after treatment and the psoriasis lesion area severity index score; and

FIG. 78 shows photos of skin lesions on the back of psoriasis-like model mice on days 0 and 7 after treatment.

### DETAILED DESCRIPTION OF THE EMBODIMENTS

The present disclosure provides a small molecule drug-oligonucleotide conjugate, which is prepared by covalent coupling a small molecule drug with an immunomodulatory function and a functional oligonucleotide molecule capable of regulating expression of an inflammation-related gene.

In the present disclosure, the covalent coupling of the small molecule drug and the functional oligonucleotide molecule is preferably conducted through a chemical linker.

The small molecule drug preferably acts on an immune-related signaling pathway and regulates an immune response; the small molecule drug is preferably selected from one or more of a calcineurin inhibitor, a glucocorticoid, a mammalian target of rapamycin (mTOR) inhibitor, and a vitamin D analog. The calcineurin inhibitor preferably includes CsA, tacrolimus, sirolimus, pimecrolimus, and their analogs; the rapamycin derivative preferably includes Temsirolimus (CCI-779) and Everolimus (RAD001); the glucocorticoid preferably includes triamcinolone acetonide (TA), dexamethasone, betamethasone, methylprednisolone, cortisone, hydrocortisone, prednisone acetate, prednisolone acetate, prednisone and their analogs; the vitamin D analog preferably includes calcipotriol (Cal), calcitriol, calcifediol, alfacalcidol, and paricalcitol.

In the present disclosure, the inflammation-related gene is one or more selected from the group consisting of a tumor necrosis factor-α (TNF-α) gene, an interleukin 1b (IL-1b) gene, an interleukin 17 (IL-17) gene, an interleukin 23 (IL-23) gene, an NFKBIZ gene, an inflammasome NLRP3 gene, a JAK gene, and a PDE4 gene. Corresponding functional oligonucleotide molecules for regulating the expression of the key genes of the inflammatory response are designed, and each gene has its corresponding functional oligonucleotide molecule for regulating the expression of the mRNA corresponding to the gene.

In the present disclosure, the functional oligonucleotide molecule is selected from one of a double-stranded siRNA, a miRNA, and a single-stranded ASO. The functional oligonucleotide molecule used can be an unmodified functional oligonucleotide molecule or a functional oligonucleotide molecule with stability enhancement modification; the stability enhancement modification preferably includes PS, 2-position OMe, MOE, and F-generation modifications.

In the present disclosure, the combinations between the small molecule drugs and the functional oligonucleotide molecules are various and not mutually restricted. One small molecule drug can be coupled with multiple functional oligonucleotide molecules, while one functional oligonucleotide molecule can also be coupled with multiple small molecule drugs to achieve different application purposes.

In the present disclosure, when the functional oligonucleotide molecule is the double-stranded siRNA or the miRNA, the small molecule drug is covalently coupled to a 3′ end of a sense strand of the functional oligonucleotide molecule; and when the functional oligonucleotide molecule is the single-stranded ASO, the small molecule drug is covalently coupled to a 3′ end or a 5′ end of the single-stranded ASO.

The small molecule drug is covalently coupled to the terminal of the functional oligonucleotide molecule or the small molecule drug is covalently coupled to a side chain base or a phosphate backbone of an extended sequence at a 3′ end or a 5′ end of the functional oligonucleotide molecule. The number of small molecule drugs covalently coupled to each functional oligonucleotide molecule is preferably any integer from 1 to 40. When the small molecule drug is covalently coupled to the terminal of the functional oligonucleotide molecule, the number of small molecule drugs covalently coupled to each functional oligonucleotide molecule is preferably any integer from 1 to 6; when the small molecule drug is covalently coupled to the side chain base or phosphate backbone of the extended sequence at the 3′ end or 5′ end of the functional oligonucleotide molecule, the number of small molecule drugs covalently coupled to each functional oligonucleotide molecule is preferably any integer from 1 to 20.

In the present disclosure, when the small molecule drug is covalently coupled to the terminal of the functional oligonucleotide molecule, the small molecule drug-oligonucleotide conjugate has a chemical structure shown in Formula 1 to Formula 14:

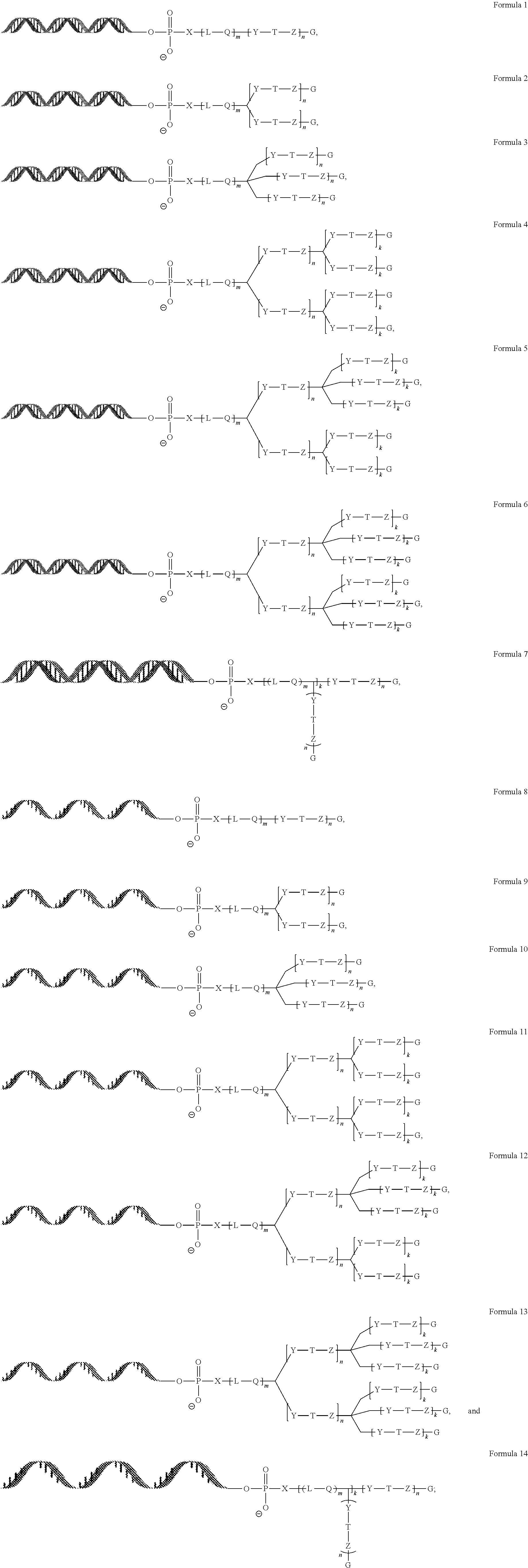

where

in Formula 1 to Formula 14, L and T are independently absent, or selected from the group consisting of —(CH₂)h- and a group formed by substituting any one or more alkylene groups in —(CH₂)h- with a group A; h is 0 to 15; the group A is selected from the group consisting of —O—, —S—, —C(O)—, —C(O)O—, —C(O)NH—, —CH(R^{C})—, —C(R′)(R″)—, —NH—, —N(R^{N})—, —S—S—, —C(R′)═C(R″)—, —C═C—,

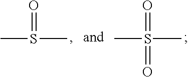

R^{C}, R^{N}, R′, and R″ in the group A represent that any one or more hydrogen atoms on a designated atom are substituted by a group B on condition that a normal valence of the designated atom is not exceeded and a stable compound is generated by substitution, and the designated atom is selected from the group consisting of a carbon atom and a nitrogen atom; the group B is selected from the group consisting of C1-C6 alkyl, C2-C6 alkenyl, C2-C6 alkynyl, cyano, hydroxyl, oxo, carboxyl, cycloalkyl, cycloalkenyl, heterocyclyl, heteroaryl, aryl, keto, alkoxycarbonyl, aryloxycarbonyl, heteroaryloxycarbonyl, and a halogen; the halogen is selected from the group consisting of F, Cl, Br, and I; and (O) in the group A represents a carbonyl oxygen atom;

in Formula 1 to Formula 14, Q, Y, and Z are independently absent, or selected from the group consisting of —O—, —S—, —C(O)—, —NH—, —CH₂—, —C(O)NH—, —NHC(O)—, —C(O)O—, —OC(O)—, —OC(O)O—, —OC(O)NH—, —NHC(O)O—, and

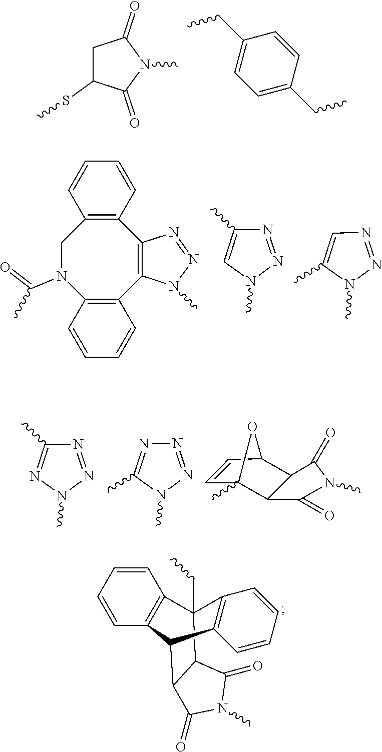

(O) in the groups Q, Y, and Z represents a carbonyl oxygen atom; and [symbol] represents a ligation site;

in Formula 1 to Formula 14, G represents a small molecule immunomodulatory drug; m, n, and k are independently 1 to 15; and X represents O or S;

preferably, when the small molecule drug is covalently coupled to the side chain base or the phosphate backbone of the extended sequence at the 3′ end or the 5′ end of the functional oligonucleotide molecule, the small molecule drug-oligonucleotide conjugate has a chemical structure shown in Formula 15 to Formula 20:

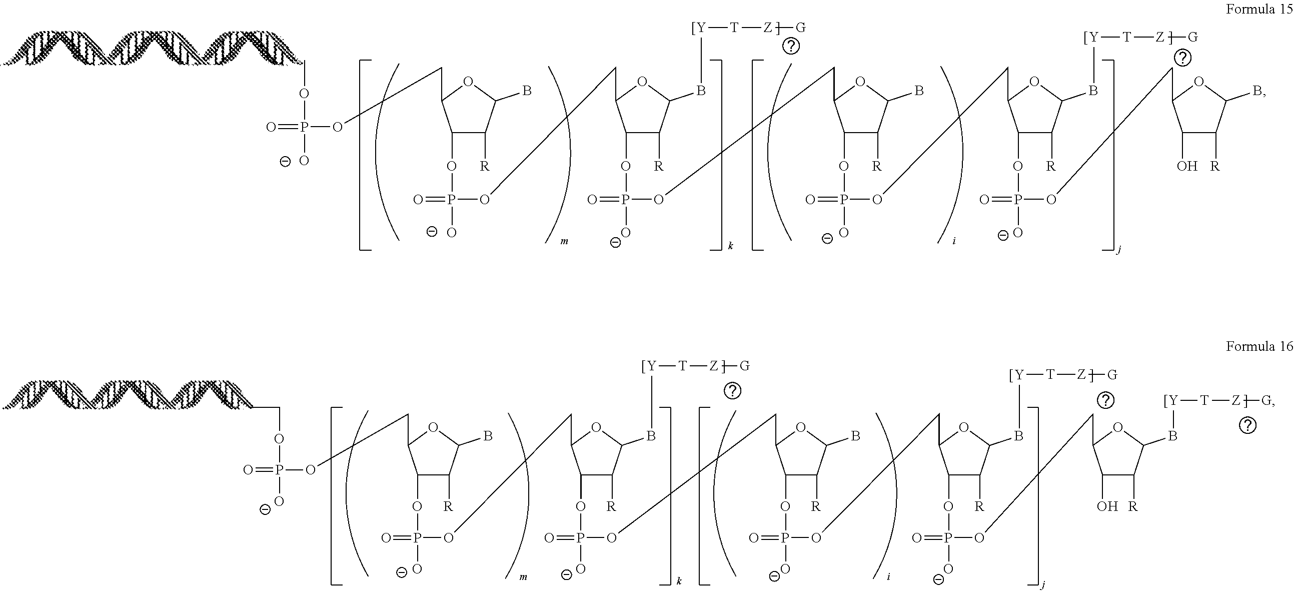

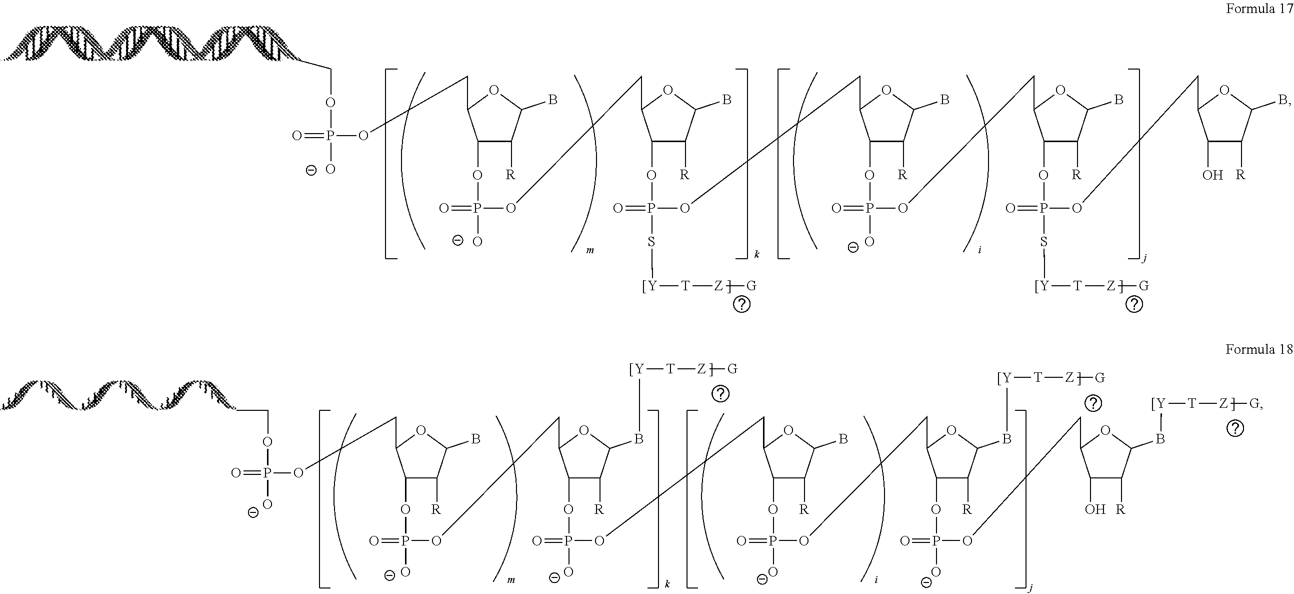

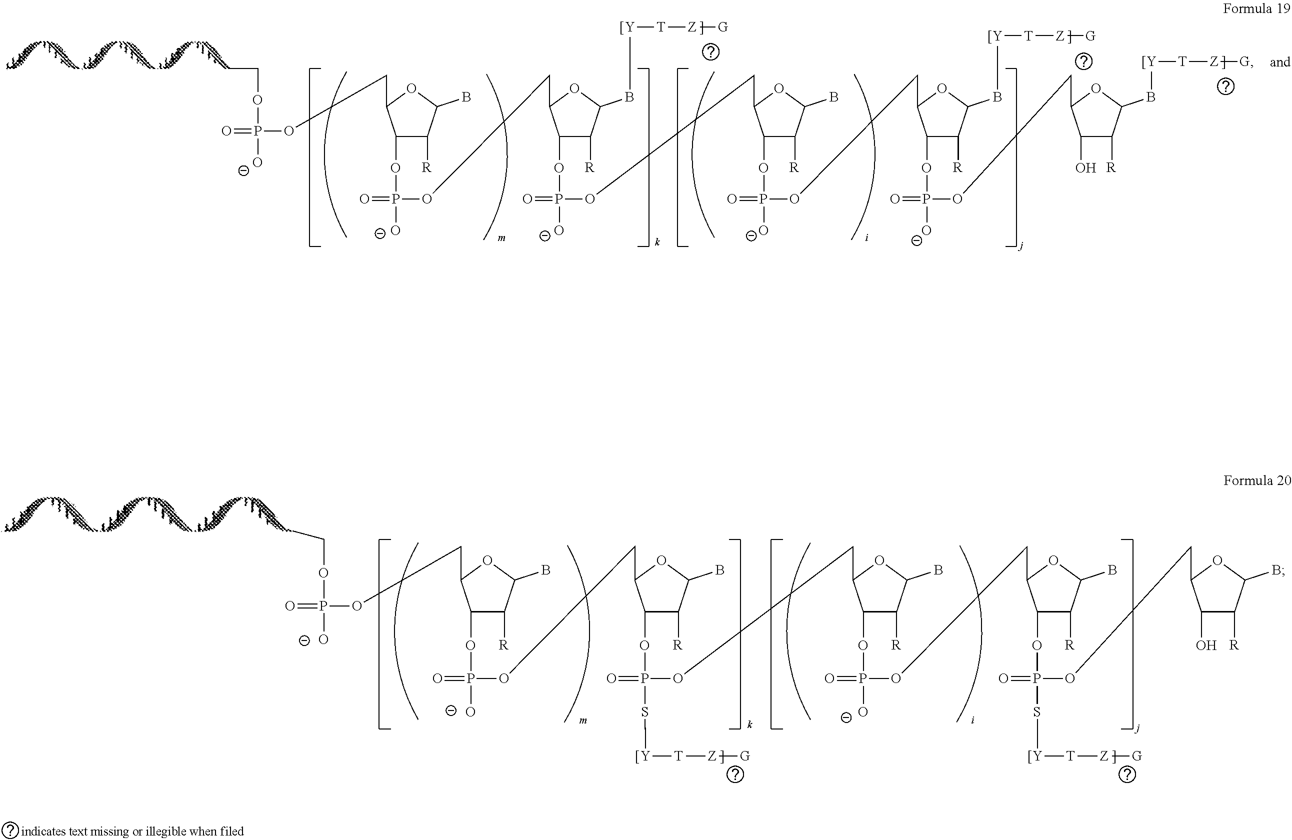

where

in Formula 15 to Formula 20, T is absent, or selected from the group consisting of —(CH₂)h- and a group formed by substituting any one or more alkylene groups in —(CH₂)h- with a group A; h is 0 to 15; (O) in the group A represents a carbonyl oxygen atom; the group A is selected from the group consisting of —O—, —S—, —C(O)—, —C(O)O—, —C(O)NH—, —CH(R^{C})—, —C(R′)(R″)—, —NH—, —N(R^{N})—, —S—S—, —C(R′)═C(R″)—, —C═C—,

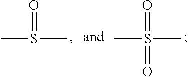

R^{C}, R^{N}, R′, and R″ in the group A represent that any one or more hydrogen atoms on a designated atom are substituted by a group B on condition that a normal valence of the designated atom is not exceeded and a stable compound is generated by substitution, and the designated atom is selected from the group consisting of a carbon atom and a nitrogen atom; the group B is selected from the group consisting of C1-C6 alkyl, C2-C6 alkenyl, C2-C6 alkynyl, cyano, hydroxyl, oxo, carboxyl, cycloalkyl, cycloalkenyl, heterocyclyl, heteroaryl, aryl, keto, alkoxycarbonyl, aryloxycarbonyl, heteroaryloxycarbonyl, and a halogen; the halogen is selected from the group consisting of F, Cl, Br, and I; and (O) in the group A represents a carbonyl oxygen atom;

in Formula 15 to Formula 20, Y and Z are independently absent or selected from the group consisting of O, S, C(O), NH, CH₂, C(O)NH, NHC(O), C(O) O, OC(O), OC(O) O, OC(O)NH, NHC(O) O, and

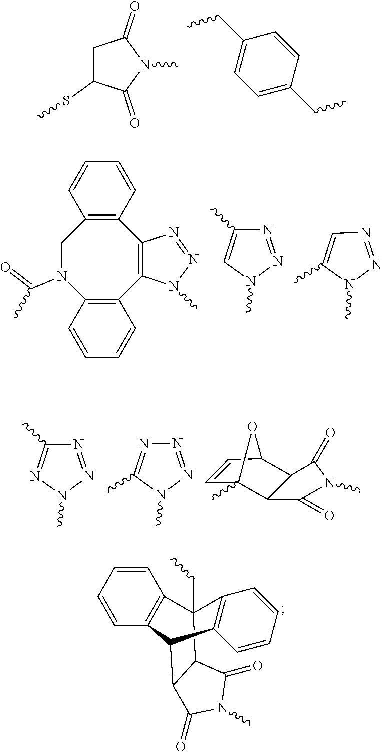

(O) in the groups Y and Z represents a carbonyl oxygen atom; and [symbol] represents a ligation site;

in Formula 15 to Formula 20, G represents an immunomodulatory inhibitor; n is 1 to 15; m and i are independently 0 to 5, and k and j are independently 1 to 20; R represents H; and B represents a nucleic acid base.

The present disclosure further provides a preparation method of the small molecule drug-oligonucleotide conjugate, including the following steps:

covalent coupling a small molecule drug with an immunomodulatory function and a functional oligonucleotide molecule capable of regulating expression of an inflammation-related gene to obtain the small molecule drug-oligonucleotide conjugate. In the present disclosure, there is no particular limitation on the specific process of covalently coupling the small molecule drug and the functional oligonucleotide molecule, and the conventional coupling process in the art can be used.

In the present disclosure, a reaction type involved in the covalent coupling preferably includes one or more of esterification, amidation, click chemistry, electrophilic substitution, nucleophilic substitution and Diels-Alder reaction.

The present disclosure further provides use of the small molecule drug-oligonucleotide conjugate in preparation of a drug for treating an inflammation-related disease.

In the present disclosure, the inflammation-related disease is one or more selected from the group consisting of xerophthalmia, psoriasis, Sjögren's syndrome, uveitis, keratitis, conjunctivitis, atopic dermatitis, rheumatoid arthritis, inflammatory bowel disease, and Crohn's disease.

In the present disclosure, a hydrophilic property of the functional oligonucleotide molecule can promote the dissolution of hydrophobic small molecule drugs with immunomodulatory function in aqueous solution, such that the drugs can be well dispersed in the buffer and efficiently taken up by cells without using other cosolvents. The increase in solubility can reduce the concentration used during administration and reduce the toxic side effects of drug molecules. At the same time, the modification of hydrophobic small molecule drugs also imparts a certain degree of hydrophobicity to the functional oligonucleotide molecules. When there is a higher number of hydrophobic drugs, the small molecule drug-oligonucleotide conjugate can be further assembled into micelles, thereby promoting the cell uptake of small molecule drug-oligonucleotide conjugate and overcoming the difficulty of nucleic acid drugs entering cells. The small molecule drug-oligonucleotide conjugate simultaneously includes a small molecule drug that regulates immune response and a functional oligonucleotide molecule. The combination of the above two components can simultaneously act on different inflammation-related signal pathways and then produce a synergistic effect small molecule drug, thereby achieving a better therapeutic effect to solve the problem of poor efficacy of small molecule immunomodulatory drugs or oligonucleotide drugs used alone, and then achieving better therapeutic effects.

In an example of the present disclosure, a CsA-NFKBIZ ASO conjugate (CsA-ASO_{NFKBIZ}) is formed by covalently coupling a single cyclosporine A (CsA) molecule to an ASO (ASO_{NFKBIZ}) targeting the nuclear factor KB inhibitor ((NFKBIZ) gene. The CsA-NFKBIZ ASO conjugate can be used to prepare a drug for treating xerophthalmia. The construction of CsA-ASO_{NFKBIZ} covalent conjugate can achieve the synergistic treatment of xerophthalmia with small molecule immunomodulatory drugs and nucleic acid drugs. CsA, as a commonly used immunity inhibitor, can inhibit the generation and release of proinflammatory cytokines by inhibiting the activity of calcineurin, and has been used clinically for the symptoms and signs of xerophthalmia. ASO targeting the NFKBIZ gene (ASO_{NFKBIZ}) can effectively knock down the expression of NFKBIZ gene in corneal epithelial cells, further reducing the level of cellular inflammation and regulating immune balance. The small molecule drug and nucleic acid drug in the CsA-ASO_{NFKBIZ} covalent conjugate may act on different targets in the inflammatory signaling pathway to achieve synergistic treatment of xerophthalmia, resulting in better therapeutic effects. The strong hydrophilicity of nucleic acid molecules endows the small molecule drug CsA high water solubility after coupling, allowing it to be efficiently taken up by cells without the help of other additives, thereby exerting its efficacy more quickly, reducing the dosage concentration during treatment, and thus minimizing the possible toxic side effects of the drug. Moreover, the prepared CsA-ASO_{NFKBIZ} covalent conjugate can enhance its interaction with cells due to the presence of hydrophobic CsA. Compared to unmodified ASO, the CsA-ASO_{NFKBIZ} conjugate can be better internalized by cells, thereby better exerting its gene regulatory effect. Finally, dual inflammatory regulatory effects of CsA and ASO achieve desirable xerophthalmia treatment effects.

In an example of the present disclosure, a small molecule drug-ASO conjugate (2CsA-ASO_{NFKBIZ}) is constructed by covalent coupling of two CsA molecules with NFKBIZ ASO (ASO_{NFKBIZ}), and its amphiphilicity promotes forming a nanomicelle assembly structure through self-assembly for co-delivery of CsA and NFKBIZ ASO; the 2CsA-ASO_{NFKBIZ} can be used to prepare drugs for treating xerophthalmia. The small molecule drug CsA and ASO drugs are used to synergistically treat xerophthalmia, and act on different targets of the inflammatory signaling pathway to synergistically reduce the level of cellular inflammation. The hydrophilic characteristics of nucleic acids can endow immune-modulating small molecule drugs better water solubility, and the hydrophobicity of CsA can enhance the interaction between the coupling product and cells, thereby improving the efficiency of drug delivery. Furthermore, compared to the CsA-ASO_{NFKBIZ} covalent conjugate prepared by coupling a single CsA molecule with an ASO targeting the NFKBIZ gene, which can be dissolved in aqueous solution in a single molecule form, the 2CsA-ASO_{NFKBIZ} covalent conjugate formed by coupling two CsA small molecules exhibits amphiphilicity due to the increase in the number of hydrophobic CsA molecules, allowing it assemble into a nanomicelle structure in the solution. The center of this assembled nanomicelle is a hydrophobic CsA molecule, and its outer shell is a water-soluble ASO molecule. The assembled nanomicelles can efficiently transfect cells without any transfection reagents, achieving efficient co-delivery of CsA and ASO, and thereby achieving a desirable synergistic therapeutic effect for xerophthalmia.

In an example of the present disclosure, a triple CsA-NFKBIZ ASO conjugate (3CsA-ASO_{NFKBIZ}) is prepared by modifying three CsA molecules to target the NFKBIZ gene. The amphiphilicity of the conjugate promotes forming a 3CsA-ASO_{NFKBIZ} nanomicelle assembly structure through self-assembly for the co-delivery of CsA and the ASO targeting the NFKBIZ gene, thereby being used for the synergistic treatment of xerophthalmia. The small molecule drug and the nucleic acid drug act simultaneously on different targets of the inflammatory signaling pathway to synergistically reduce the level of cellular inflammation. The hydrophilicity of nucleic acids can endow the immunomodulatory small molecule drug better water solubility, and the hydrophobicity of CsA can enhance the interaction between the conjugate product and cells, thereby improving the utilization efficiency of the drug. Compared to the ASO conjugates of a single or two CsA molecules with ASO targeting the NFKBIZ gene, the drug-nucleic acid conjugate 3CsA-ASO_{NFKBIZ} with 3 CSA small molecules has stronger amphiphilicity after coupling with nucleic acids and is easier to assemble into nanomicelles in the solution, such that the 3CsA-ASO_{NFKBIZ} nanomicelles are easier to interact with cells, achieving a better synergistic treatment effect for xerophthalmia.

In an example of the present disclosure, a CsA-ASO conjugate (CsA₃-ASO_{NFKBIZ}) formed by covalently coupling a CsA trimer molecule with an ASO targeting NFKBIZ (ASO_{NFKBIZ}) is constructed, and spherical nanomicelles formed by their self-assembly are explored for the synergistic treatment of xerophthalmia. Three CsA molecules are grafted onto the terminal of ASO through click chemistry, and its purification and preparation methods are simple and controllable. The strong hydrophobicity of the three CsA molecules makes the conjugate have strong amphiphilicity, allowing the prepared conjugated product to self-assemble into a stable nanomicelle structure in aqueous solution, with a low critical micelle concentration. The spherical nucleic acid micelle nanostructure can be efficiently taken up by cells, realizing the simultaneous co-delivery of CsA and ASO. As an immunomodulator and anti-inflammatory drug, CsA can inhibit the generation and release of pro-inflammatory cytokines and increase the release level of anti-inflammatory cytokines, effectively improving the symptoms and signs of xerophthalmia caused by long-term inflammation in clinical practice. At the same time, ASO can knock down the expression of inflammation-related gene NFKBIZ, further inhibiting the inflammatory response of xerophthalmia. Through synergistic regulation, the inflammatory response is inhibited, the immune balance is regulated, and better xerophthalmia treatment effects are achieved.

In an example of the present disclosure, single, double, and triple CsA are covalently coupled to siRNA targeting the NFKBIZ gene (siRNA_{NFKBIZ}) to form drug-nucleic acid conjugates, and the prepared conjugates or their assembled nanostructures are used to further achieve synergistic treatment of xerophthalmia. In the present disclosure, the small molecule drug and nucleic acid drug synergistically treat xerophthalmia. CsA is an immunomodulator and anti-inflammatory drug that can inhibit the generation and release of pro-inflammatory cytokines, and can effectively improve the symptoms and signs of xerophthalmia caused by long-term inflammation in clinical practice. At the same time, NFKBIZ siRNA can knock down the expression of NFKBIZ in corneal epithelial cells, further reducing the level of cell inflammation. The hydrophilic characteristics of siRNA can endow CsA drugs better water solubility, reducing the concentration used during systemic application, and thus reducing toxic side effects. At the same time, the hydrophobic immunomodulatory small molecule drug CsA makes the conjugate of the above two easier to interact with cells, improving the RNA drug's ability to enter cells, thereby achieving a better treatment effect for xerophthalmia. This example is intended to expand the class of drug-nucleic acid conjugates in the treatment of xerophthalmia.

In an example of the present disclosure, a drug-ASO conjugate is constructed by modifying multiple CsA molecules by phosphorothioate grafting to target the NFKBIZ gene (ASO_{NFKBIZ}), and its amphiphilicity promotes self-assembly to form nanomicelles for synergistic treatment of xerophthalmia. This structure can not only achieve the synergistic treatment of xerophthalmia by CsA and ASO_{NFKBIZ}, but also enhance the water solubility of CsA and the stability of nucleic acid drugs, improving the ability of drug-nucleic acid conjugates to interact with cells. At the same time, the ASO_{NFKBIZ} structure grafted with 20 CsA molecules has a smaller CMC value when assembled into nanomicelles, carries more therapeutic small molecule drugs, and can achieve better therapeutic effects.

In an example of the present disclosure, small molecule drug-nucleic acid conjugates and their self-assembled nanomicelles are prepared by covalently coupling and modifying three CsA molecules with siRNAs targeting IL-17 (siRNA_{IL-1}), IL-1B (siRNA_{IL-1β}), IL-23 (siRNA_{IL-23}), and TNF-α (siRNA_{TNF-α}) are constructed for the synergistic treatment of xerophthalmia. Triple CsA and siRNA drug-nucleic acid conjugate nanomicelles targeting different inflammatory factors are prepared for the synergistic treatment of xerophthalmia. The selected gene targets are different inflammatory factors secreted extracellularly by cells, which further verifies the multi-target feasibility of this structure in the treatment of xerophthalmia.

In an example of the present disclosure, small molecule drug-nucleic acid conjugates and their self-assembled nanomicelles prepared by covalently coupling and modifying three CsA molecules with siRNAs targeting NLRP3 (siRNA_{NLRP3}), JAK1 (siRNA_{JAK1}), and PDE4 (siRNA_{PDE4}) are constructed for the synergistic treatment of xerophthalmia. A conjugate of siRNA targeting key genes related to intracellular inflammation and CsA molecules is prepared, and its amphiphilicity promotes assembling into nanomicelles for the synergistic treatment of xerophthalmia. Different inflammatory gene targets within the cell are selected to further verify the multi-target feasibility of this structure in the treatment of xerophthalmia.

In an example of the present disclosure, self-assembled nanomicelles of drug nucleic acid conjugates co-delivered by 3CsA and ASO_{IL-17}, 3CsA and ASO_{IL-18}, 3CsA and ASO_{IL-23}, and 3CsA and ASO_{TNF-α} are constructed for the synergistic treatment of xerophthalmia. Triple CsA and ASO drug-nucleic acid conjugate nanomicelles targeting different inflammatory factors are prepared for the synergistic treatment of xerophthalmia. The selected gene targets are different inflammatory factors secreted extracellularly by cells, which further verifies the multi-target feasibility of this structure in the treatment of xerophthalmia.

In an example of the present disclosure, self-assembled nanomicelles of drug-nucleic acid conjugates co-delivered by 20CsA and ASO_{NLRP3}, 20CSA and ASO_{JAK1}, and 20CsA and ASO_{PDE4} are constructed for the synergistic treatment of xerophthalmia. The 20PS-modified ASO is grafted with multiple CsA small molecules to increase the drug loading while reducing the therapeutic dose, thus improving the safety of treatment. At the same time, multiple intracellular inflammatory therapeutic targets in xerophthalmia are selected to further explore the feasibility of multi-targets of inflammatory drug-nucleic acid conjugates in the treatment of xerophthalmia.

In an example of the present disclosure, a small molecule drug-ASO conjugate (10RAP-ASO_{NFKBIZ}) obtained by modifying ASO targeting NFKBIZ gene with 10 rapamycin molecules (RAP) and its self-assembled nanomicelles are constructed for the synergistic treatment of xerophthalmia. The rapamycin-ASO conjugate constructed by grafting multiple rapamycin molecules on the nucleic acid backbone at a terminal of the ASO sequence has a high drug loading capacity. The hydrophobicity of rapamycin molecules and the hydrophilicity of nucleic acid molecules endow the rapamycin-ASO conjugate with amphiphilic characteristics, which can self-assemble in aqueous solution to form nanomicelle structures. The nanomicelle structure can be effectively taken up by cells without other carrier materials, thereby achieving efficient co-delivery of rapamycin and ASO; the rapamycin-ASO conjugate can achieve combined treatment of xerophthalmia by regulating different inflammation-related signal pathways. Rapamycin, as an mTOR inhibitor, can effectively inhibit the mTOR signal transduction pathway to achieve inflammation inhibition. At the same time, ASO targeting the NFKBIZ gene can effectively knock down the expression of the NFKBIZ gene, further inhibiting the inflammatory response associated with xerophthalmia. Through the synergistic action of the above two, the inflammatory response is inhibited, thereby achieving a desirable treatment effect of xerophthalmia.

In an example of the present disclosure, a small molecule drug-ASO conjugate formed by covalent coupling of CsA trimer (CsA₃) and ASO targeting NFKBIZ (ASO_{NFKBIZ}) and a spherical nanomicelle formed by self-assembly are constructed for the synergistic treatment of psoriasis. Three CsA molecules are grafted onto the terminal of ASO through click chemistry, and the purification and preparation methods are simple and controllable. The strong hydrophobicity of the three CsA molecules imparts strong amphiphilicity of the conjugate, allowing it to self-assemble into a stable nanomicelle structure in aqueous solution without a carrier. The prepared spherical micelle nucleic acid nanostructure can be efficiently taken up by cells, realizing the simultaneous co-delivery of CsA and ASO. CsA, as an immunomodulator and anti-inflammatory drug, can inhibit the generation and release of pro-inflammatory cytokines and increase the release level of anti-inflammatory cytokines. At the same time, ASO can knock down the expression of the inflammation-related gene NFKBIZ, further inhibiting the inflammatory response of psoriasis. Through synergistic regulation, the inflammatory response is inhibited, the immune balance is regulated, and better psoriasis treatment effects are achieved.

In an example of the present disclosure, a small molecule drug-ASO conjugate modified with 10 TA molecules targeting the NFKBIZ gene and the nanomicelle nanostructure formed by their assembly are constructed for the synergistic treatment of psoriasis. The TA-ASO conjugate constructed by grafting multiple TA molecules at the terminal of the ASO sequence has a high drug loading capacity. The hydrophobicity of TA and the hydrophilicity of nucleic acid molecules can endow the TA-ASO conjugate amphiphilicity, allowing it to self-assemble into a nanomicelle structure in an aqueous solution. The nanomicelle structure can achieve efficient co-delivery of TA and ASO without other carrier materials, and can be effectively taken up by cells. The combined treatment of psoriasis is achieved by regulating different inflammation-related signal pathways. TA, as a glucocorticoid, has anti-inflammatory, anti-itching, and vasoconstriction effects, and shows strong and lasting anti-inflammatory and anti-allergic effects in the treatment of skin diseases. ASO targeting the NFKBIZ gene can effectively knock down NFKBIZ protein expression, further inhibiting the psoriasis-related inflammatory responses. Through the synergistic action of the above two, the inflammatory response is inhibited, thereby achieving a desirable psoriasis treatment effect.

In an example of the present disclosure, a small molecule drug-ASO conjugate (10Cal-ASO_{NFKBIZ}) prepared by covalently coupling 10 Cal molecules with an ASO targeting the NFKBIZ gene and the nanomicelles formed by their assembly are constructed for the synergistic treatment of psoriasis. The Cal-ASO conjugate constructed by grafting multiple Cal molecules at the terminal of the ASO sequence has a high drug loading capacity. The hydrophobicity of Cal and the hydrophilicity of nucleic acid molecules can endow the TA-ASO conjugate amphiphilicity, allowing it to self-assemble into a nanomicelle structure in an aqueous solution. The nanomicelle structure can achieve efficient co-delivery of Cal and ASO without other carrier materials, and can be effectively taken up by cells. The combined treatment of psoriasis is achieved by regulating different inflammation-related signaling pathways, where the Cal is an analog of vitamin D3, and can inhibit the proliferation of skin cells (keratinocytes) and induce their differentiation, thereby correcting the hyperplasia and differentiation abnormalities of psoriasis lesions. ASO targeting the NFKBIZ gene can effectively knock down NFKBIZ protein expression, further inhibiting psoriasis-related inflammatory responses. Through the synergistic action of the above two, the inflammatory response is inhibited, thereby achieving a desirable psoriasis treatment effect.

Unless otherwise specified, the present disclosure has no special requirements for sources of the used raw materials, and commercially-available products well known to those skilled in the art may be adopted.

The present disclosure will be further described below with reference to specific examples. It should be understood that these examples are only intended to describe the present disclosure, rather than to limit the scope of the present disclosure. Therefore, improvement and adjustment made by those skilled in the art based on the above contents of the present disclosure still belong to the protection scope of the present disclosure.

### Example 1 Synergistic Treatment of Xerophthalmia with Conjugate of CsA and ASO Targeting Nuclear Factor KB Inhibitor ξ (NFKBIZ)

### 1.1 Synthesis of Azidohexanoic Acid Carbonate-Modified CsA (CsA-N₃)

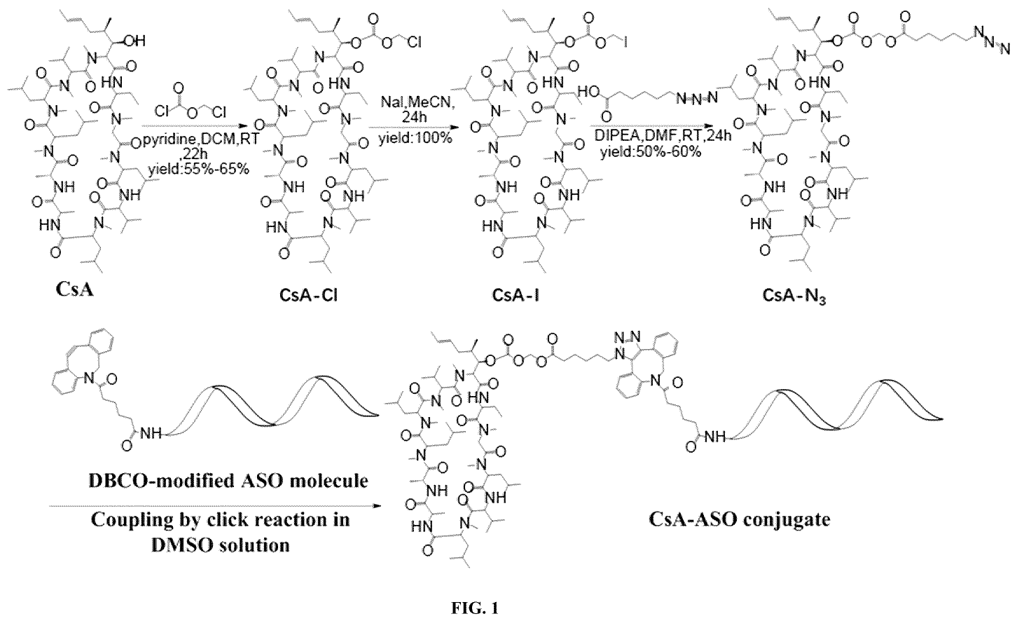

In this example, the preparation routes of azidohexanoic acid carbonate-modified CsA (CsA-N₃) and CsA-ASO_{NFKBIZ} covalent conjugates were shown in FIG. 1.

### 1.1.1 Synthesis of Chloromethyl Carbonate CsA (CsA-C1)

Under nitrogen protection, CsA (1,000 mg) was placed in a dry 50 mL round-bottom flask, 10 mL of anhydrous dichloromethane (DCM) was added and stirred until dissolved, the reaction bottle was placed in an ice bath, and then 592 μL of chloromethyl chloroformate was added dropwise, and then 336 μL of anhydrous pyridine was added dropwise, and a resulting mixture was naturally restored to room temperature and stirred to allow reaction for 24 h. After the reaction was completed, 40 mL of anhydrous DCM was added to dilute the reaction solution, and the mixture was washed 3 times with 50 mL of saturated sodium bicarbonate solution and one time with 50 mL of saturated sodium chloride solution. The organic phase was collected and dried over anhydrous sodium sulfate. The organic phase was concentrated by vacuum distillation and then separated and purified by silica gel column chromatography with an eluent of petroleum ether/ethyl acetate. Finally, 550 mg of a white solid was obtained, namely the CsA-Cl with a yield of 60%.

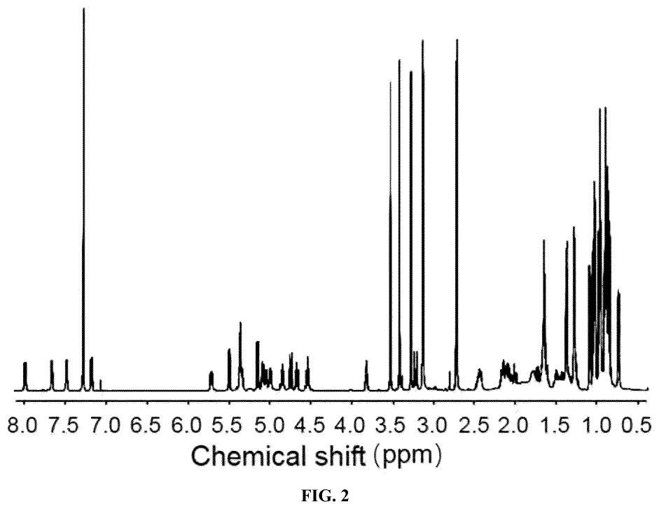

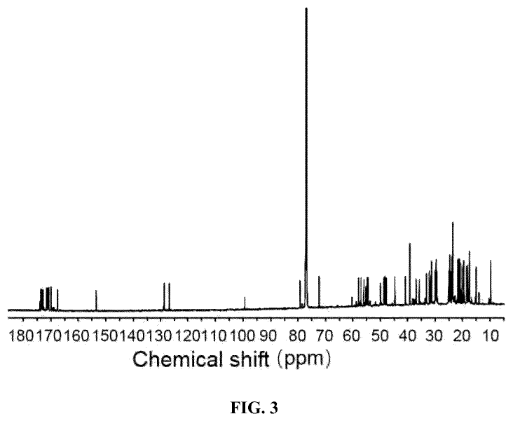

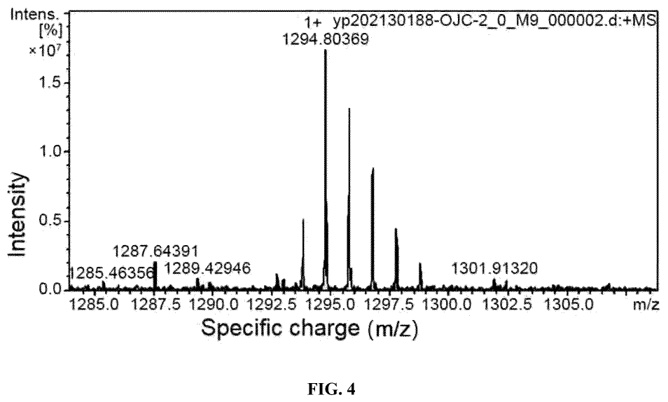

The ¹H NMR spectrum of CsA-Cl was shown in FIG. 2, and a solvent used in the nuclear magnetic spectrum test was CDCl₃. The attribution of each proton peak was as follows: δ (ppm): 8.46 (1H, d), 7.91 (1H, d), 7.46 (1H, d), 7.37 (1H, d), 5.72 (1H, d), 5.62 (1H, dd), 5.47 (1H, d), 5.31 (2H, m), 5.21 (2H, m), 5.06 (2H, m), 5.00 (1H, q), 4.88 (1H, t), 4.77 (1H, t), 4.70 (1H, t), 4.60 (1H, d), 4.33 (1H, t), 4.05 (1H, q), 3.39 (3H, s), 3.25 (3H, s), 3.16 (3H, s), 3.13 (1H, s), 3.01 (3H, d), 2.60 (3H, d), 2.34 (1H, m), 2.06 (4H, m), 2.00 (4H, m), 1.92 (2H, m), 1.81 (3H, m), 1.66 (6H, m), 1.53 (4H, d), 1.42 (2H, m), 1.34 (1H, m), 1.26 (3H, d), 1.20 (3H, d), 1.14 (1H, dq), 1.05 (3H, d), 1.04 (1H, d), 0.98 (3H, t), 0.94 (3H, d), 0.91 (3H, d), 0.89 (3H, d), 0.88 (3H, d), 0.86 (3H, d), 0.82 (9H, m), 0.81 (3H, d), 0.76 (3H, d). The ¹³C NMR spectrum of CsA-Cl was shown in FIG. 3, and a solvent used in the nuclear magnetic spectrum test was CDCl₃. The attribution of the characteristic carbon contained in the product was as follows: 173.7, 173.4, 173.1, 172.8, 171.6, 171.2, 170.9, 170.8, 170.0, 169.9, 167.6, 153.5. The theoretical molecular weight of CsA-Cl was 1,293.80, and the molecular weight actually measured by matrix-assisted laser desorption ionization time-of-flight mass spectrometry (MALDI-TOF) was 1,294.80, which was attributed to the molecular ion peak [M+1]⁺ of CsA-Cl, as shown in FIG. 4.

### 1.1.2 Synthesis of Iodomethyl Carbonate CsA (CsA-I)

Under nitrogen protection, CsA-Cl (500 mg) was placed in a dry 25 mL round-bottom flask, 1 mL of anhydrous DCM was added and stirred to dissolve, DCM was removed by vacuum distillation under nitrogen flow, 5 mL of anhydrous acetonitrile was added to dissolve, sodium iodide (500 mL) was added to the reaction solution, and the reaction was conducted at 40° C. for 24 h. After the reaction was completed, acetonitrile was removed by vacuum distillation, and then 50 mL of anhydrous DCM was added. The mixture was washed 2 times with 50 mL of saturated sodium chloride solution, the organic phase was collected, and then dried over anhydrous sodium sulfate. The solvent was removed by vacuum distillation and then vacuum dried in a vacuum drying oven (40° C.) to obtain CsA-I, which was directly used in the next step.

### 1.1.3 Synthesis of Azidohexanoic Acid Carbonate-Linked CsA (CsA-N₃)

Under nitrogen protection, the prepared CsA-I (200 mg) was placed in a 10 mL round-bottom flask and dissolved with 1 mL of anhydrous DMF. 40 mg of 6-azidohexanoic acid was dissolved in 1 mL of anhydrous DMF and added dropwise to CsA-I. 60 μL of anhydrous N,N-diisopropylethylamine (DIPEA) was added and the mixture was reacted at room temperature for 24 h. After the reaction was completed, DMF was removed by vacuum distillation, and the reaction product was dissolved in 50 mL of ethyl acetate, washed 2 times with 50 mL of 10% sodium metabisulfite solution (w/v), washed 1 time with a saturated sodium chloride solution, and then the organic phase was collected, dried over anhydrous sodium sulfate, and the organic phase was filtered and concentrated by vacuum distillation, and then separated and purified by silica gel column chromatography with an eluent of petroleum ether/ethyl acetate (1:2), and finally about 120 mg of a white solid product (Rf: 0.2) was collected, namely the azidohexanoic acid carbonate-modified CsA.

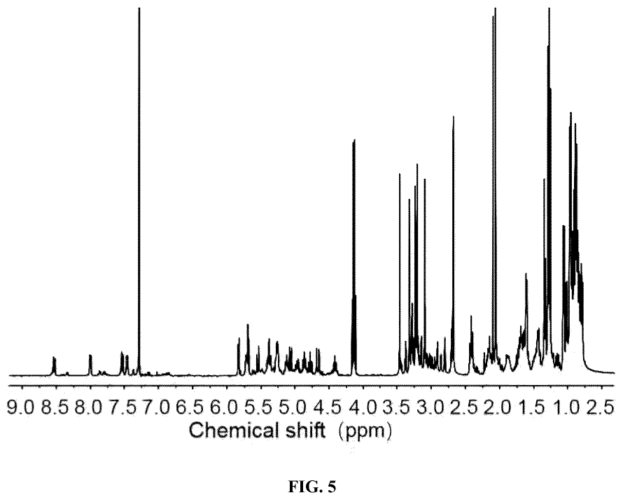

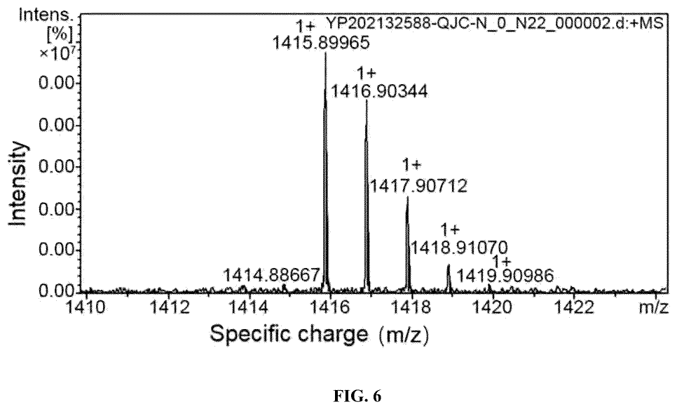

The ¹H NMR spectrum of CsA-N₃ was shown in FIG. 5, and a solvent used in the nuclear magnetic spectrum test was CDCl₃. The attribution of each proton peak was as follows: δ (ppm): 8.54 (1H, d), 7.98 (1H, d), 7.52 (1H, d), 7.46 (1H, d), 5.82 (1H, d), 5.70 (1H, dd), 5.68 (1H, d), 5.54 (1H, d), 5.37 (2H, m), 5.25 (2H, m), 5.11 (2H, m), 5.06 (1H, d), 4.97 (1H, t), 4.85 (1H, t), 4.77 (1H, t), 4.66 (1H, d), 4.40 (1H, t), 3.46 (3H, s), 3.31 (3H, s), 3.28 (2H, d), 3.23 (3H, s), 3.20 (3H, s), 3.09 (1H, s), 2.67 (3H, d), 2.41 (1H, t), 2.12 (4H, m), 2.06 (4H, m), 1.99 (2H, m), 1.87 (3H, m), 1.66 (6H, m), 1.60 (4H, d), 1.42 (2H, m), 1.33 (3H, d), 1.26 (3H, d), 1.20 (3H, d), 1.14 (1H, dq), 1.05 (7H, d), 1.00 (3H, d), 0.96 (3H, t), 0.94 (12H, m), 0.91 (3H, d), 0.89 (3H, d), 0.88 (3H, d), 0.86 (3H, d), 0.82 (9H, m), 0.81 (3H, d), 0.76 (3H, d). The theoretical molecular weight of azidohexanoic acid carbonate-modified CsA was 1,415.89, and the actual measured molecular weight was 1,416.90, which was attributed to the molecular ion peak [M+1]⁺ of azidohexanoic acid carbonate-modified CsA, as shown in FIG. 6.

### 1.2 Synthesis of CsA and NFKBIZ ASO Covalent Conjugate (CSA-ASO_{NFKBIZ})

In this example, the ASO targeting the NFKBIZ gene was purchased from Shanghai Sangon Biotech Co., Ltd., and dibenzocyclooctyne (DBCO) modification was introduced at the 3′ end of the ASO to enable efficient coupling with the azide group modified on CsA-N₃ through a click reaction. The 3′ end DBCO group modification was represented by -DBCO-3′ in all the following examples, and its modified structure was shown below:

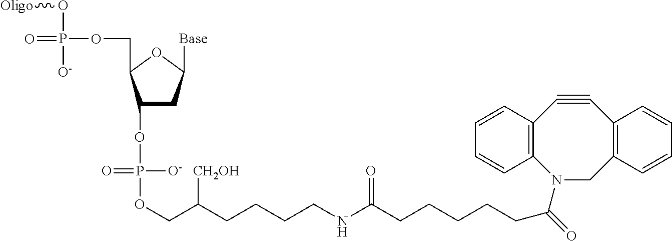

The Nucleic Acid Sequences Used in this Example were as Follows:

the sequence of the ASO targeting human NFKBIZ gene (DBCO-ASO_{NFKBIZ-h}) was:

(SEQ ID NO: 1)
Human: 5′-ATCAGACAACGAATCGGGC-DBCO-3′;

the sequence of the ASO targeting mouse NFKBIZ gene (DBCO-ASO_{NFKBIZ-m}) was:

(SEQ ID NO: 2)
Mouse: 5′-AATACTGGTACATTGACGCC-DBCO-3′.

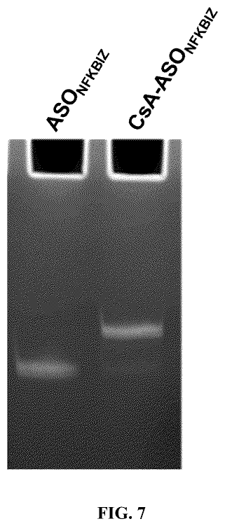

The synthesis method of covalently modifying NFKBIZ ASO with a single CsA molecule to prepare a covalent conjugate (CsA-ASO_{NFKBIZ}) included: 0.2 mg of CsA-N₃ (130 nmol) was dissolved in 0.13 mL of DMSO, 5 OD of DBCO-ASO_{NFKBIZ} (26 nmol) was added, and a reaction was conducted by shaking at 50° C. for 24 h. 5 mL of water was added, and the excess CsA-N₃ in the reaction was removed by extraction with ethyl acetate, and then the aqueous solution was concentrated and evaporated to dryness to obtain the CsA-ASO_{NFKBIZ} conjugate molecule. The successful coupling of CsA and ASO was verified by 10% denatured polyacrylamide gel, as shown in FIG. 7. There was a high coupling efficiency of single CsA and DBCO-ASO_{NFKBIZ}, about 95%.

### 1.3 Cellular Uptake of CsA-ASO_{NFKBIZ-h} Conjugate

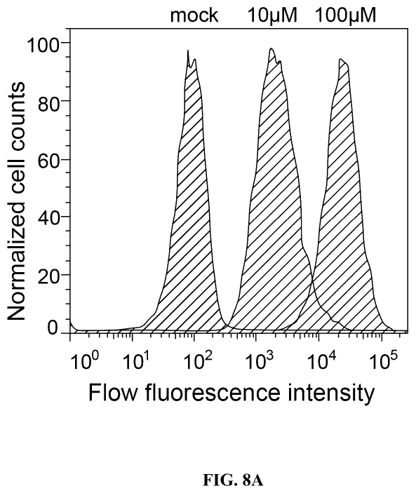

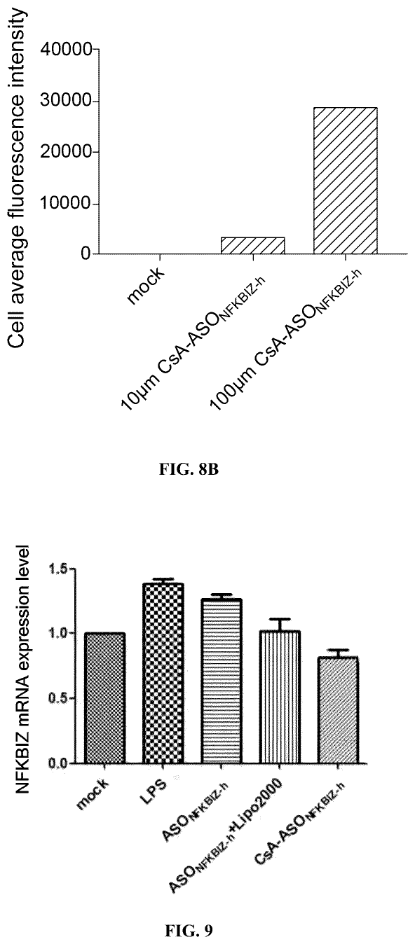

Human corneal epithelial cells (HCECs) were inoculated at a density of 5×10⁴ cells/well in a 12-well plate and cultured overnight. After the medium was removed, 0.5 mL of fresh medium containing 10 μM or 100 μM CsA-ASO_{NFKBIZ-h} conjugate with FITC fluorescence was added and incubated with HCECs for 6 h, while a well plate without conjugate was used as a blank control (Mock group). After the incubation, the supernatant medium was discarded, and then the cells were digested with trypsin and washed 2 to 3 times with phosphate-buffered saline (PBS, pH=7.2). After the cells were collected, flow cytometric analysis was conducted using a flow cytometer, and experimental results were shown in FIGS. 8A-8B. The results showed that the CsA-ASO_{NFKBIZ-h} conjugate could be effectively taken up by cells, and the cells showed obvious fluorescence enhancement signals after incubation. The average fluorescence intensity of cells after incubation with 100 UM CsA-ASO_{NFKBIZ}-h conjugate was approximately 10 times that after co-incubation with 10 μM, demonstrating that the CsA-ASO_{NFKBIZ-h} conjugate had a desirable intracellular effect.

### 1.4 Regulatory Effect of CsA-ASO_{NFKBIZ-h} Conjugate on Target Gene NFKBIZ

HCECs were inoculated in a 6-well plate at a density of 1×10⁵ cells/well and cultured until adherence. The cells were then stimulated with 100 ng/mL lipopolysaccharide (LPS) overnight. After removing the supernatant, 1 mL of Opti-MEM medium containing ASO_{NFKBIZ-h}, ASO_{NFKBIZ-h}+Lipofectamine2000 (Lipo2000), and CsA-ASO_{NFKBIZ-h} (all containing 10 UM ASO) were added, respectively. Cells without any treatment were used as a blank reference (Mock). After incubation at 37° C. for 6 h, the cells were transferred into DMEM medium and incubated for another 48 h. The cells were collected and RNA was extracted for RT-qPCR to analyze the expression level of the NFKBIZ mRNA in HCECs, and experimental results were shown in FIG. 9. The expression level of NFKBIZ mRNA in HCECs after LPS stimulation was about 1.3 times that of normal cells. ASO_{NFKBIZ-h} had a certain inhibitory effect on the expression of NFKBIZ mRNA in human ocular epithelial cells after LPS stimulation without transfection reagent, but it was still about 1.25 times that of normal cells. After ASO_{NFKBIZ-h} was transfected with Lipo2000, its expression level was reduced to that of normal cells. After adding CsA-ASO_{NFKBIZ-h} for co-incubation, the expression of NFKBIZ mRNA was reduced to about 0.8 times that of normal cells. This indicated that the knockdown ability of the CsA-ASO_{NFKBIZ-h} conjugate on NFKBIZ mRNA was better than that of the unmodified ASO_{NFKBIZ-h}.

### 1.5 Therapeutic Effect of CsA-ASO_{NFKBIZ-m} Conjugate on Xerophthalmia

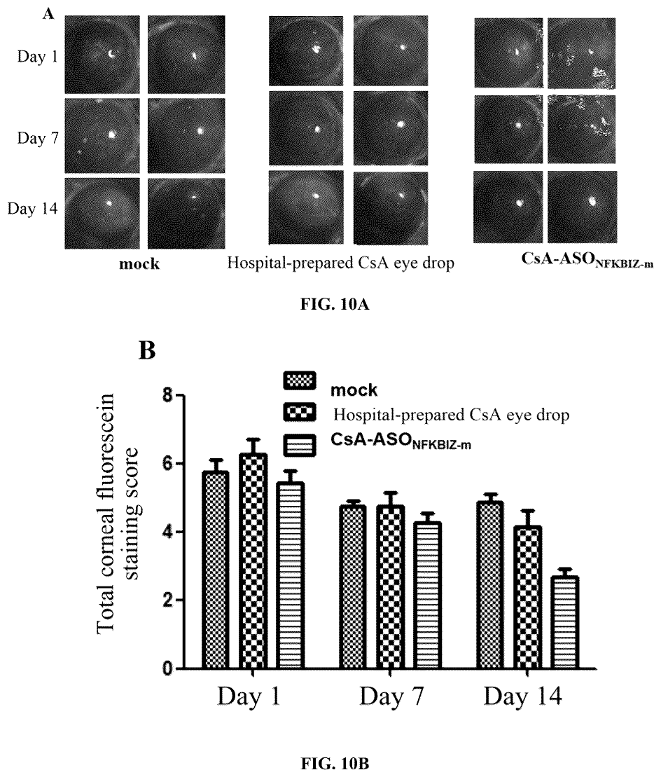

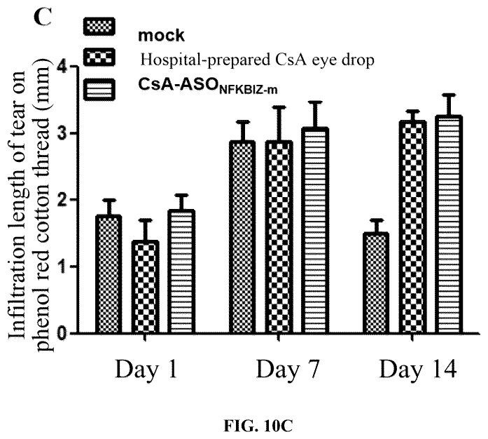

The ocular xerophthalmia model was established in 6-8 weeks old mice (C57BL/6, Suzhou Xishan Biotechnology Co., Ltd.) with 0.2% benzalkonium chloride. 5 μL of 0.2% benzalkonium chloride was dripped into each eye of the mice, once in the morning and once in the evening every day, for two consecutive weeks. After successful modeling, the hospital-prepared CsA eye drops (400 UM CsA, referred to as hospital-prepared CsA eye drops or CsA eye drops, provided by the Eye, Ear, Nose and Throat Hospital Affiliated to Fudan University School of Medicine, Shanghai) and CsA-ASO_{NFKBIZ-m} (400 UM CsA) were used for eye drops treatment, 5 μL per eye, once in the morning and once in the evening. Sodium fluorescein staining and phenol red cotton thread tear secretion test were conducted on days 0, 7, and 14 of treatment to evaluate the therapeutic effect. The experimental results were shown in FIGS. 10A-10C.

The results of sodium fluorescein staining showed that the eye score of mice in the hospital-prepared CsA eye drops treatment group decreased on the 7th day; and after 14 d of treatment, the score dropped from slightly above 6 points to about 5 points, and the phenol red cotton thread infiltration length increased from 1.5 mm to about 3 mm. The sodium fluorescein score of mice in the CsA-ASO_{NFKBIZ-m} treatment group dropped from 5.5 points to about 3 points after 14 d, and the phenol red cotton thread infiltration length increased from 1.8 mm to about 3.2 mm. Compared with the pathology group and the simple hospital-prescribed CsA eye drops group, CsA-ASO_{NFKBIZ-m} could better achieve the delivery of small molecule drugs and nucleic acid drugs. The above two could synergistically inhibit ocular surface inflammation in mice with xerophthalmia, showing a better therapeutic effect on xerophthalmia in mice.

### Example 2 Synergistic Treatment of Xerophthalmia with Covalent Conjugate of Double CsA-Modified NFKBIZ ASO (2CSA-ASO_{NFKBIZ}) and its Assembly Structure

### 2.1 Preparation and Characterization of 2CsA-ASO_{NFKBIZ} Conjugate and its Self-Assembled Nanomicelles

In this example, the ASO targeting the NFKBIZ gene was purchased from Shanghai Sangon Biotech Co., Ltd., and 2 dibenzocyclooctyne (DBCO) modifications were introduced at the 3′ end of the ASO to enable efficient coupling with the azide group modified on CsA-N₃ through a click reaction. The first DBCO modification was the same as in Example 1 and was located at the 3′ end of the nucleic acid sequence; the second DBCO modification was close to the 3′ end and was a modification of the middle T base of the nucleic acid sequence. In all the following examples, the T base containing the middle modified DBCO was represented by the symbol/iDBCOdT/, and its structure was shown below:

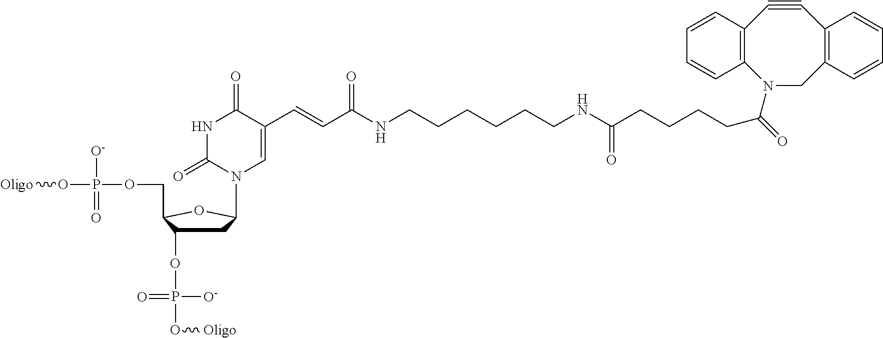

The nucleic acid sequences used in this example were as follows:

the sequence of the ASO targeting human NFKBIZ gene (2DBCO-ASO_{NFKBIZ-h}) was:

(SEQ ID SEQ: 3)
Human: 5′-ATCAGACAACGAATCGGGC/iDBCOdT/TT-DBCO-3′;

the sequence of the ASO targeting mouse NFKBIZ gene (2DBCO-ASO_{NFKBIZ-m}) was:

(SEQ ID SEQ: 4)
Mouse: 5′-AATACTGGTACATTGACGCC/iDBCOdT/TT-DBCO-3′.

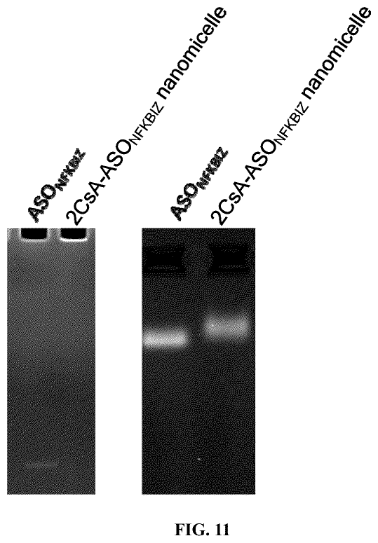

The synthesis method of 2CsA-ASO_{NFKBIZ} conjugate was as follows: 0.4 mg of CSA-N₃ (260 nmol) prepared in Example 1 was dissolved in 0.13 mL of DMSO, 5 OD of 2DBCO-ASO_{NFKBIZ} (26 nmol) was added, and the mixture was reacted by shaking at 50° C. for 24 h. After adding 5 mL of water, the excess CsA-N₃ in the reaction was extracted with ethyl acetate, and the 2CsA-ASO_{NFKBIZ} conjugate molecule was obtained after concentration and evaporation, and redissolved with 50 μL of DMSO solution and then dropped into 500 μL of PBS for self-assembly to form 2CsA-ASO_{NFKBIZ} nanomicelles. The DMSO was then removed by dialyzing in PBS solution to obtain the final self-assembled nucleic acid nanomicelles. The successful grafting coupling of CsA and ASO and the successful assembly of nanomicelles were verified by 10% denatured polyacrylamide gel electrophoresis and 1% agarose gel electrophoresis, as shown in FIG. 11. The efficiency of CsA grafting 2DBCO-ASO_{NFKBIZ} was high, with a yield of more than 95%. At the same time, the size of the 2CsA-ASO_{NFKBIZ} nanomicelles formed after assembly was relatively uniform, appearing as a single band in the 1% agarose gel electrophoresis diagram.

### 2.2 Regulatory Effect of 2CsA-ASO_{NFKBIZ-h} Nanomicelles on the Target NFKBIZ Gene

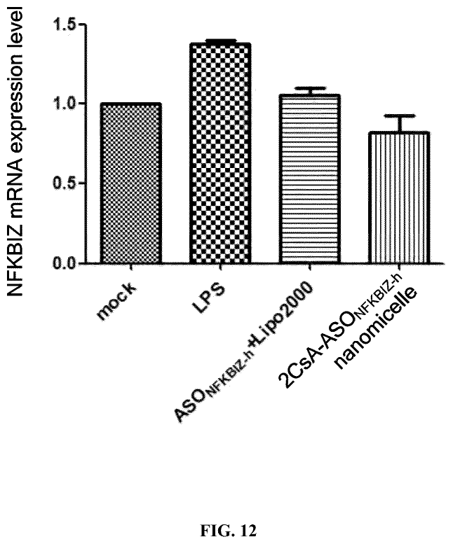

HCECs were inoculated in a 6-well plate at a density of 1×10⁵ cells/well and cultured until adherence. The cells were then stimulated with 100 ng/mL LPS overnight. After removing the supernatant, 1 mL of Opti-MEM medium containing ASO_{NFKBIZ-h}+Lipo2000 complex and 2CsA-ASO_{NFKBIZ-h} nanomicelles (all containing 10 UM ASO) were added, respectively. After incubation at 37° C. for 6 h, the cells were transferred into DMEM medium and incubated for another 48 h. The cells were collected and RNA was extracted for RT-qPCR to analyze the expression level of the NFKBIZ mRNA in HCECs, and experimental results were shown in FIG. 12.

It can be seen from FIG. 12 that the expression of NFKBIZ mRNA in HCECs stimulated by LPS increased to about 1.4 times that of normal cells, and ASO_{NFKBIZ-h} after Lipo2000 transfection could downregulate it to the level of normal cells. After co-incubation with 2CsA-ASO_{NFKBIZ-h} nanomicelles, the expression of NFKBIZ mRNA in LPS-stimulated HCECs decreased to about 0.8 times that of normal cells. In summary, compared to the single-targeted NFKBIZ ASO, the 2CsA-ASO_{NFKBIZ-h} nanomicelles had a stronger knockdown ability on the expression of NFKBIZ gene.

### 2.3 Therapeutic Effect of 2CsA-ASO_{NFKBIZ-m} Nanomicelles on Xerophthalmia

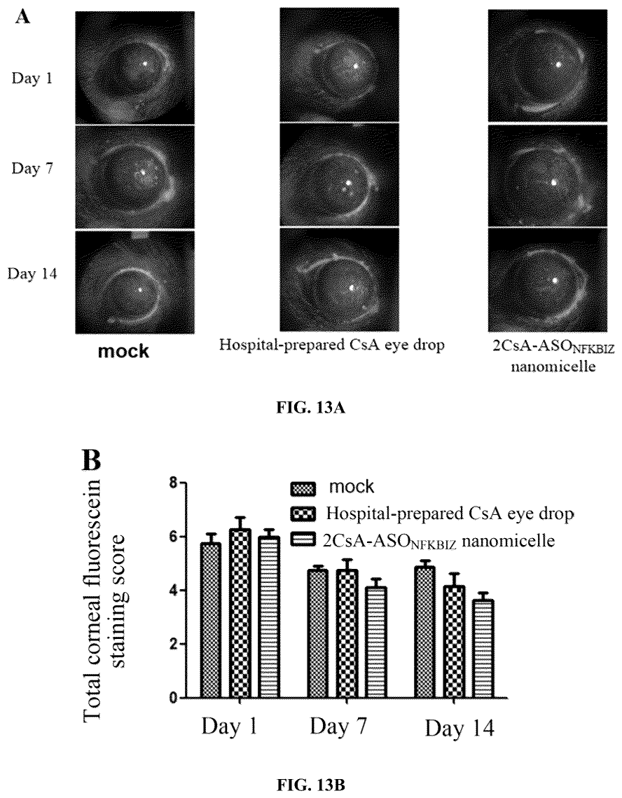

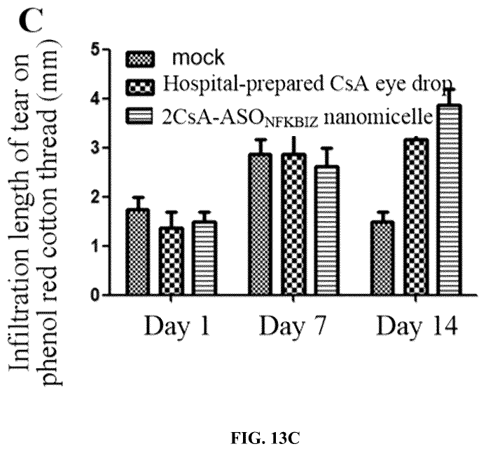

The ocular xerophthalmia model was established in 6-8 weeks old mice (C57BL/6, Suzhou Xishan Biotechnology Co., Ltd.) with 0.2% benzalkonium chloride. 5 μL of 0.2% benzalkonium chloride was dripped into each eye of the mice, once in the morning and once in the evening every day, for two consecutive weeks. After successful modeling, the hospital-prepared CsA eye drops (400 μM CsA, provided by the Eye, Ear, Nose and Throat Hospital Affiliated to Fudan University School of Medicine, Shanghai) and 2CsA-ASO_{NFKBIZ-m} (400 UM CsA) were used for eye drops treatment, 5 μL per eye, once in the morning and once in the evening. Sodium fluorescein staining and phenol red cotton thread tear secretion test were conducted on days 0, 7, and 14 of treatment to evaluate the therapeutic effect. The experimental results were shown in FIGS. 13A-13C.

The results of sodium fluorescein staining showed that after treatment with the hospital-prepared CsA eye drops, the mouse eye score dropped from 6 points to about 4 points, and the corresponding infiltration length of tear on phenol red cotton thread increased from 1.5 mm to about 3 mm. After treatment with 2CsA-ASO_{NFKBIZ-m} nanomicelles, the sodium fluorescein staining score of the mouse eyes dropped from 6 points to about 3.5 points, and the infiltration length of phenol red cotton thread increased from 1.5 mm to about 3.5 mm. The results of animal experiments showed that compared to the case group and the hospital-prescribed CsA eye drops treatment group, the 2CsA-ASO_{NFKBIZ-m} nanomicelles had a more significant therapeutic effect on xerophthalmia in mice. This is due to the spherical nucleic acid nanomicelles formed by self-assembly having better cell-entry ability; and the small molecule drugs and nucleic acid drugs being able to achieve synergistic treatment, thereby better inhibiting inflammation in mouse eye tissues and achieving the purpose of xerophthalmia treatment.

### Example 3 Synergistic Treatment of Xerophthalmia with Triple CsA-Modified NFKBIZ ASO Covalent Conjugate (3CsA-ASO_{NFKBIZ}) and its Assembly Structure

### 3.1 Preparation and Characterization of 3CsA-ASO_{NFKBIZ} Conjugate and its Self-Assembled Nanomicelles

In this example, the ASO targeting the NFKBIZ gene was purchased from Shanghai Sangon Biotech Co., Ltd., and 3 dibenzocyclooctyne (DBCO) modifications were introduced at the 3′ end of the ASO to enable efficient coupling with the azide group modified on CsA-N₃ through a click reaction. The first DBCO modifications was located at the 3′ end of the nucleic acid sequence; the other two were DBCO modification of the middle T base of the nucleic acid sequence, close to the 3′ end, and their sequences were as follows:

the sequence of the ASO targeting human NFKBIZ gene (3DBCO-ASO_{NFKBIZ-h}) was:

Human:
(SEQ ID SEQ: 5)
5′-ATCAGACAACGAATCGGGC/iDBCOdT/T/iDBCOdT/TT-
DBCO-3′;

the sequence of the ASO targeting mouse NFKBIZ gene (3DBCO-ASO_{NFKBIZ-m}) was:

Mouse:
(SEQ ID SEQ: 6)
5′-AATACTGGTACATTGACGCC/iDBCOdT/T/iDBCOdT/TT-
DBCO-3′.

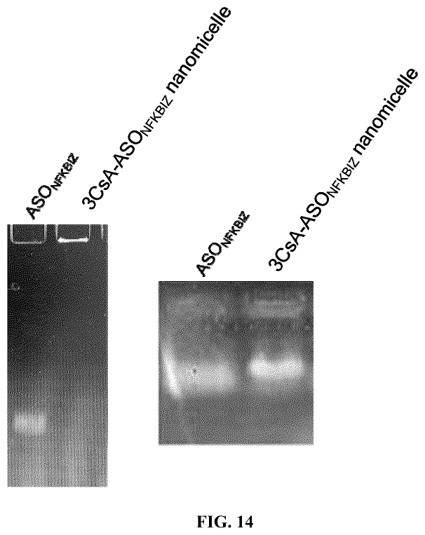

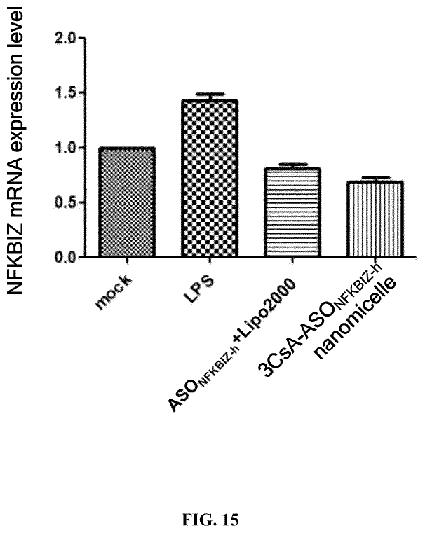

The synthesis method of 3CsA-ASO_{NFKBIZ} conjugate was as follows: 0.6 mg of CSA-N₃ (390 nmol) was dissolved in 0.13 mL of DMSO, 5 OD of DNA (26 nmol) was added, and the mixture was reacted by shaking at 50° C. for 24 h. After adding 5 mL of water, the excess CsA-N₃ in the reaction was extracted with ethyl acetate, and the 3CsA-ASO_{NFKBIZ} conjugate molecule was obtained after concentration and evaporation, and redissolved with 50 μL of DMSO solution and then dropped into 500 μL of PBS for self-assembly to form 3CsA-ASO_{NFKBIZ} nanomicelles. The DMSO was then removed by dialyzing in PBS solution to obtain the final self-assembled nucleic acid nanomicelles. The successful grafting coupling and the successful assembly were verified by 10% denatured polyacrylamide gel electrophoresis and 1% agarose gel electrophoresis, as shown in FIG. 14. There was a high efficiency of CsA in grafting 3DBCO-ASO_{NFKBIZ}, and a relatively uniform size of the assembled nanomicelles.

### 3.2 Regulatory Effect of 3CsA-ASO_{NFKBIZ-h} Nanomicelles on the Target NFKBIZ Gene

HCECs were inoculated in a 6-well plate at a density of 1×10⁵ cells/well and cultured until adherence. The cells were then stimulated with 100 ng/ml LPS overnight. After removing the supernatant, 1 mL of Opti-MEM medium containing ASO_{NFKBIZ-h}+Lipo2000 complex and 3CsA-ASO_{NFKBIZ-h} nanomicelles (all containing 10 UM ASO) were added, respectively. After incubation at 37° C. for 6 h, the cells were transferred into DMEM medium and incubated for another 48 h. The cells were collected and RNA was extracted for RT-qPCR to analyze the expression level of the NFKBIZ mRNA in HCECs, and experimental results were shown in FIG. 15.

It can be seen from FIG. 15 that the expression of NFKBIZ mRNA in HCECs stimulated by LPS increased to about 1.4 times that of normal cells, and ASO_{NFKBIZ-h} after Lipo2000 transfection could downregulate it to the level of normal cells. 3CsA-ASO_{NFKBIZ-h} nanomicelles down-regulated the NFKBIZ gene to about 0.75 times that of normal cells. Therefore, compared to a single ASO, 3CsA-ASO_{NFKBIZ-h} nanomicelles had a better ability to down-regulate the expression of the NFKBIZ gene.

### 3.3 Therapeutic Effect of 3CsA-ASO_{NFKBIZ-m} Nanomicelles on Xerophthalmia

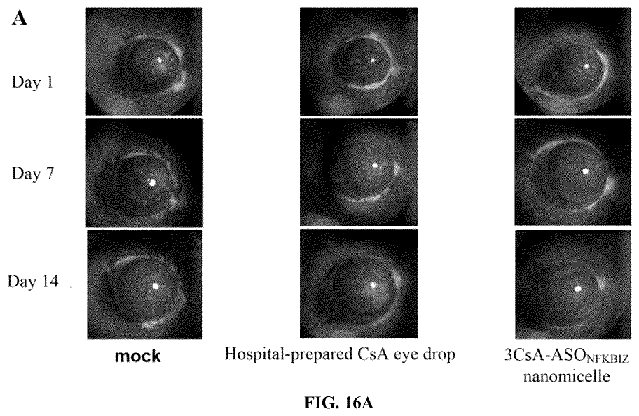

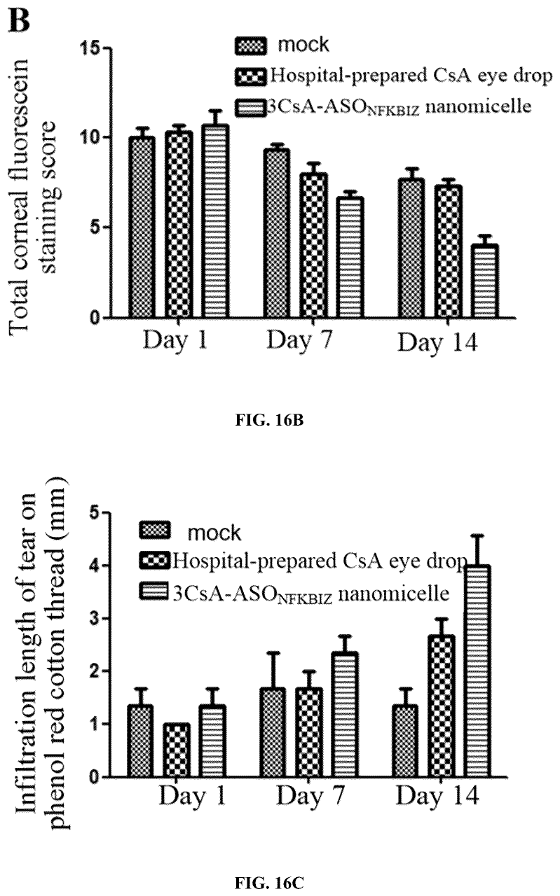

The ocular xerophthalmia model was established in 6-8 weeks old mice (C57BL/6, Suzhou Xishan Biotechnology Co., Ltd.) with 0.2% benzalkonium chloride. 5 μL of 0.2% benzalkonium chloride was dripped into each eye of the mice, once in the morning and once in the evening every day, for two consecutive weeks. After successful modeling, the hospital-prepared CsA eye drops (400 UM CsA, provided the Eye, Ear, Nose and Throat Hospital Affiliated to Fudan University School of Medicine, Shanghai) and 3CsA-ASO_{NFKBIZ-m} nanomicelles (133 UM ASO concentration, 400 UM CsA) were used for eye drops treatment, 5 μL per eye, once in the morning and once in the evening. Sodium fluorescein staining and phenol red cotton thread tear secretion test were conducted on days 0, 7, and 14 of treatment to evaluate the therapeutic effect. The experimental results were shown in FIGS. 16A-16C.

The results of sodium fluorescein staining showed that the eye score of mice treated with hospital-prepared CsA eye drops dropped from 10 points to about 8 points, and the infiltration length of phenol red cotton thread increased from 1 mm to about 2.5 mm. The sodium fluorescein score of mice after 3CsA-ASO_{NFKBIZ-m} nanomicelle treatment dropped from 10 points to about 4 points, and the phenol red cotton thread infiltration length increased from 1.3 mm to about 4 mm. The results of animal experiments showed that compared to the case group and the simple CsA eye drops group, the nanomicelles assembled from the conjugation of 3 CsA molecules with ASO targeting NFKBIZ had a more significant therapeutic effect on xerophthalmia in mice.

### Example 4 the Treatment of Xerophthalmia with CsA Trimer (CsA₃)-Modified NFKBIZ ASO covalent conjugate (CsA₃-ASO_{NFKBIZ}) and its Nanomicelle Assembly Structure

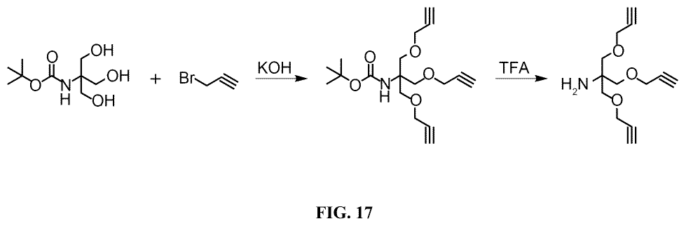

### 4.1 Synthesis of Linker, Aminotris [(2-Propynyloxy)Methyl]Methane, as Shown in FIG. 17

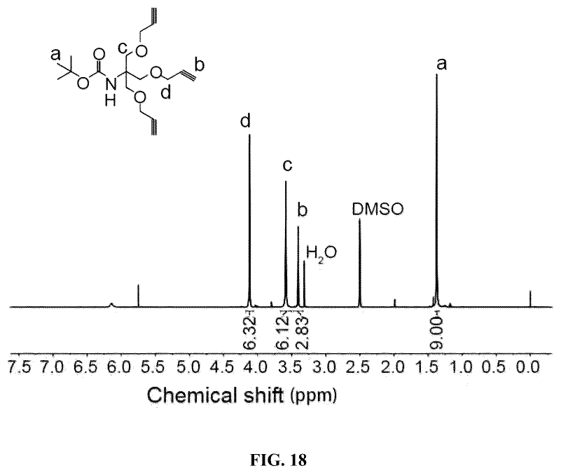

a) Synthesis of Boc-aminotris [(2-propynyloxy)methyl]methane: 1.0 g of Boc-aminotris (hydroxymethyl) methane (4.52 mmol, 1 eq.) was dissolved in 10 mL of anhydrous DMF. The mixture was stirred in an ice bath, and 3.2 g of propargyl bromide (27.12 mmol, 6 eq.) was added. 1.5 g of potassium hydroxide powder (27.12 mmol, 6 eq.) was added to the mixture in 3 batches within 15 min, the reaction solution was heated to 35° C., and the reaction was continued for 24 h under a nitrogen atmosphere. 100 mL of ethyl acetate was added to the reaction solution, and the solution was washed 3 times with 200 mL of deionized water. The organic phase of ethyl acetate was collected by extraction, dried over anhydrous sodium sulfate, and then the mixture was evaporated to dryness by rotary evaporation. The product was purified by silica gel column chromatography to obtain the Boc-aminotris [(2-propynyloxy)methyl]methane. The eluent was a mixed solution of n-hexane/ethyl acetate. The yield of Boc-aminotris(propargyl) methane was 64%. The NMR spectrum of the product and the peak attribution were shown in FIG. 18.
b) Synthesis of aminotris [(2-propynyloxy)methyl]methane: 0.5 g of the Boc-aminotris [(2-propynyloxy)methyl]methane was dissolved in 5 mL of anhydrous DCM. The mixture was placed in an ice-water bath and stirred, and 3 mL of trifluoroacetic acid (TFA) was slowly added dropwise to the mixture and continued stirring for about 2 h. After the reaction, the solvent was evaporated using a rotary evaporator, and then 15 mL of saturated sodium bicarbonate solution was added, and 50 mL of ethyl acetate was added for extraction 3 times, and the organic layers were collected and combined, washed with 15 mL of deionized water, the organic phase was collected again and dried over anhydrous sodium sulfate, and the solvent was evaporated by rotary evaporation to finally obtain the aminotris [(2-propynyloxy)methyl]methane with a yield of 98%.

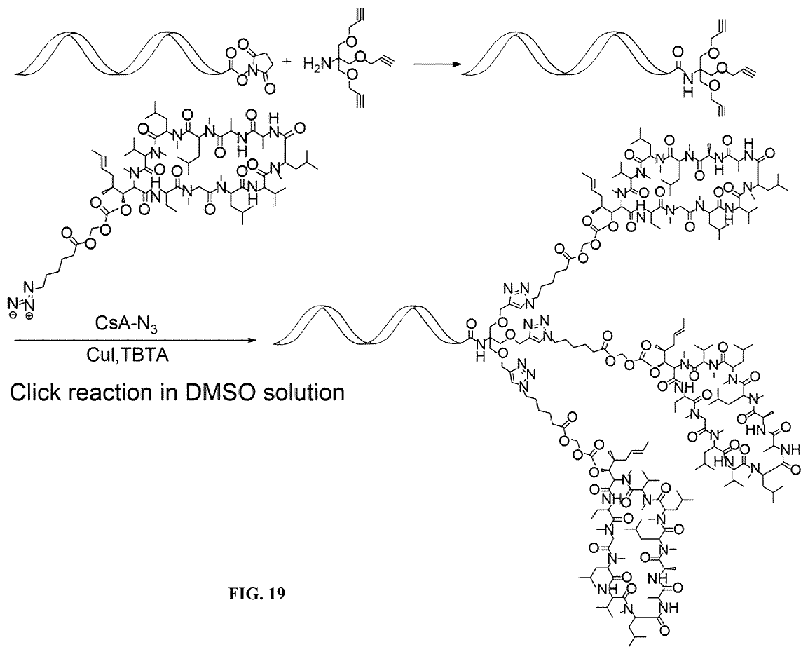

### 4.2 Synthesis of CsA Trimer-ASO Conjugate (CsA₃-ASO), as Shown in FIG. 19

In this example, the ASO targeting the NFKBIZ gene was purchased from Shanghai Sangon Biotech Co., Ltd., and N-hydroxysuccinimide ester (NHS ester) functional group was introduced at the 3′ end of the ASO, such that it could be coupled with the aminotris [(2-propynyloxy)methyl]methane to obtain ASO with three alkynyl groups at the 3′ end to enable efficient coupling with the azide group modified on CsA-N₃ through a click reaction. The 3′ end NHS group modification was represented by -NHS-3′ in all the following examples, and its modified structure was shown below:

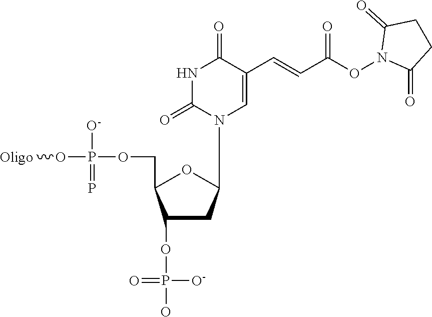

The nucleic acid sequences used in this example were as follows:

the sequence of the ASO targeting human NFKBIZ gene (NHS-ASO_{NFKBIZ-h}) was:

(SEQ ID NO: 7)
Human: 5′-ATCAGACAACGAATCGGGC-NHS-3′;

the sequence of the ASO targeting mouse NFKBIZ gene (NHS-ASO_{NFKBIZ-m}) was:

(SEQ ID NO: 8)
Mouse: 5′-AATACTGGTACATTGACGCC-NHS-3′.

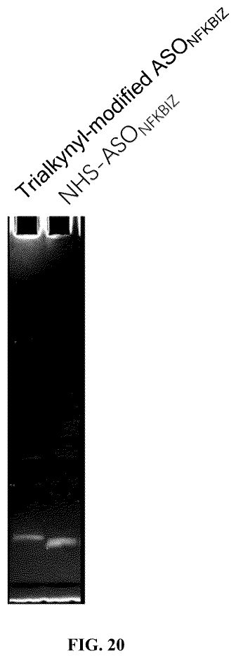

The synthesis method of CsA₃-ASO conjugate included:
a) 1.20 mg of the aminotris [(2-propynyloxy)methyl]methane (5.12 μmol, 50 eq.) was dissolved in 800 μL of DMSO, and 20 OD of NHS-modified ASO (NHS-ASO, 102.40 nmol, 1 eq.) was dissolved in 200 μL of 1×PBS buffer. The DMSO and 1×PBS buffer solution were fully mixed according to the volume ratio of 4:1 and stirred at room temperature overnight. After adding deionized water, the excess aminotris [(2-propynyloxy)methyl]methane in the reaction was removed by extraction with ethyl acetate, and the tripropargyl-modified ASO conjugate molecule was obtained after concentration and evaporation. The successful grafting of the conjugate molecules was verified by 20% denatured polyacrylamide gel electrophoresis at 600 V for 2 h in 1×TBE buffer, as shown in FIG. 20.

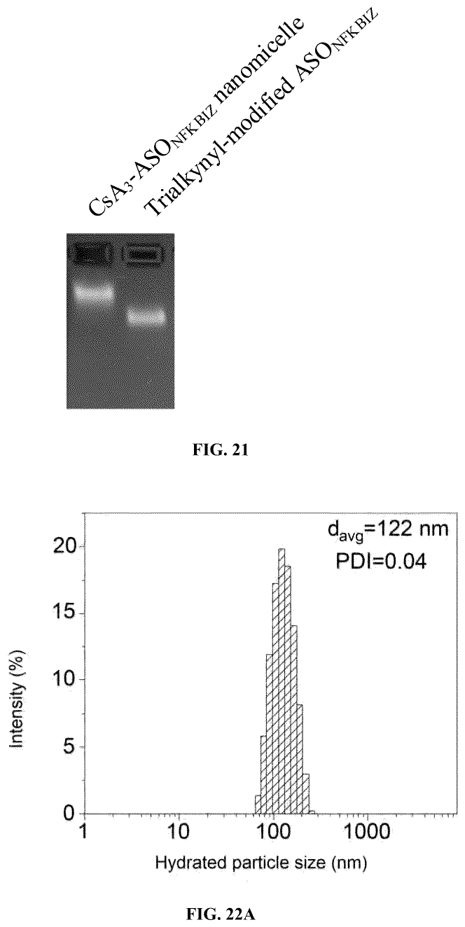

b) 1.09 mg of azidohexanoic acid carbonate-linked CsA (CsA-N₃, 0.77 μmol, 15 eq.) and 0.56 mg of Cul. TBTA (0.77 μmol, 15 eq.) were dissolved in 475 μL of DMF, and 10 OD of the tripropargyl-modified ASO conjugate molecule (51.20 nmol, 1 eq.) was dissolved in 25 μL of sterile water. After DMF and water were fully mixed at a volume ratio of 95:5, the reaction was conducted by stirring overnight under nitrogen protection. The product was concentrated in vacuum to a trace volume and purified by high-performance liquid chromatography (HPLC) to obtain the CsA trimer-ASO conjugate (CsA₃-ASO_{NFKBIZ}). The successful grafting was verified by 0.5% agarose gel electrophoresis running at 80 V for 20 min in 1×TAE buffer, as shown in FIG. 21.

4.3 Preparation and Characterization of CsA₃-ASO_{NFKBIZ} Nanomicelles

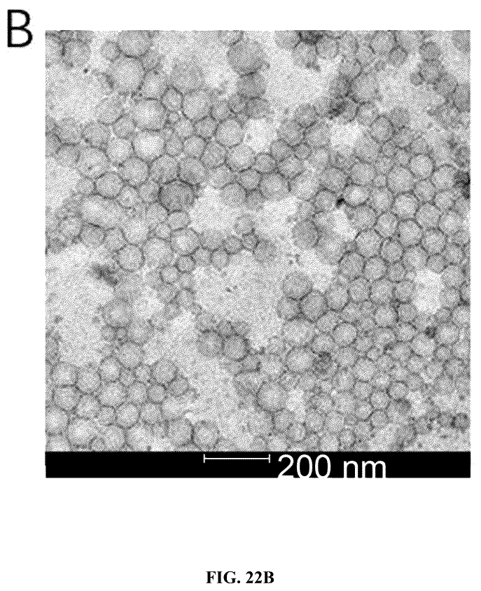

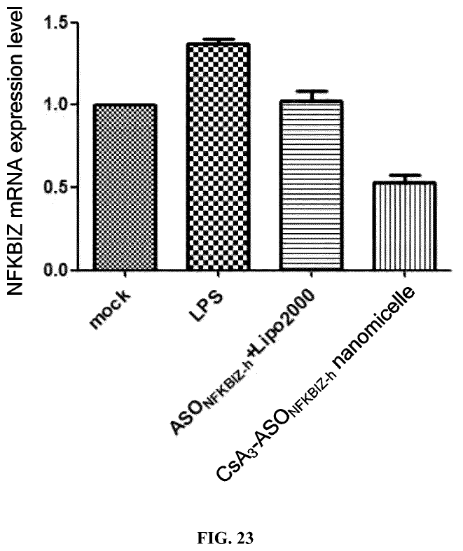

The CsA₃-ASO_{NFKBIZ} conjugate (10 OD ASO) was dissolved in 100 μL of DMSO and added dropwise to 300 μL of 1×PBS buffer with continuous stirring. The solution was dialyzed overnight in a dialysis bag to remove DMSO. CsA₃-ASO_{NFKBIZ} nanomicelles were obtained by centrifugation at 3,000 rpm for 5 min. The diameter and morphology of nanomicelles were characterized by DLS and TEM, as shown in FIGS. 22A-22B.

4.4 Regulatory Effect of CsA₃-ASO_{NFKBIZ-h} Nanomicelles on NFKBIZ Gene

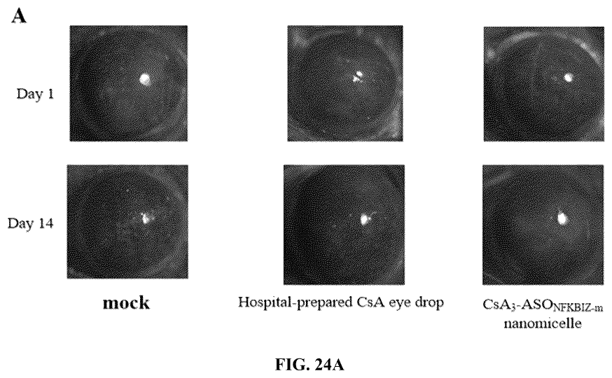

HCECs were inoculated in a 6-well plate at a density of 1×10⁵ cells/well and cultured until adherence. The cells were then stimulated with 100 ng/mL LPS overnight. 1 mL of Opti-MEM medium containing ASO_{NFKBIZ-h}+Lipo2000 complex and CsA₃-ASO_{NFKBIZ-h} nanomicelles (all containing 10 UM ASO) were added, respectively. After incubation at Ltd. 37° C. for 6 h, the cells were transferred into DMEM medium and incubated for another 48 h. After collected and RNA was extracted for RT-qPCR to evaluate the expression level of NFKBIZ mRNA in HCECs, and the experimental results were shown in FIG. 23.

It can be seen from FIG. 23 that the expression level of NFKBIZ mRNA in HCECs after LPS stimulation was about 1.5 times that of normal cells. After ASO was transfected with Lipo2000, its expression level was reduced to that of normal cells. After adding CsA₃-ASO_{NFKBIZ-h} nanomicelles for co-incubation, the expression of NFKBIZ mRNA was reduced to about 0.5 times that of normal cells. The CsA₃-ASO_{NFKBIZ-h} nanomicelles had a significant downregulation ability on NFKBIZ mRNA.

4.5 Therapeutic Effect of CsA₃-ASO_{NFKBIZ-m} Nanomicelles on Xerophthalmia

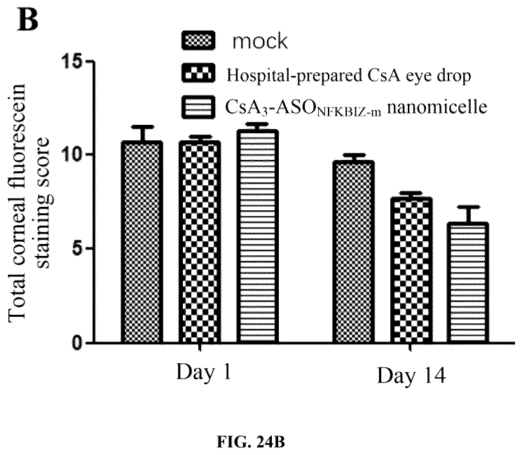

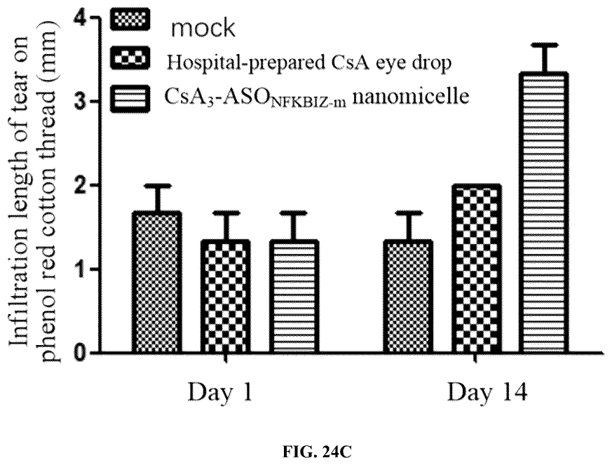

The ocular xerophthalmia model was established in 6-8 weeks old mice (C57BL/6, Suzhou Xishan Biotechnology Co., Ltd.) with 0.2% benzalkonium chloride. 5 μL of 0.2% benzalkonium chloride was dripped into each eye of the mice, once in the morning and once in the evening every day, for two consecutive weeks. After successful modeling, the hospital-prepared CsA eye drops (400 μM CsA, provided by the Eye, Ear, Nose and Throat Hospital Affiliated to Fudan University School of Medicine, Shanghai) and CsA₃-ASO_{NFKBIZ-m} nanomicelles (400 UM CsA) were used for eye drops treatment, 5 μL per eye, once in the morning and once in the evening. Sodium fluorescein staining and phenol red cotton thread tear secretion test were conducted on days 0 and 14 of treatment to evaluate the therapeutic effect. The experimental results were shown in FIGS. 24A-24C.

The results of sodium fluorescein staining showed that the eye score of mice treated with hospital-prepared CsA eye drops dropped from 10 points to about 7.5 points, and the infiltration length of phenol red cotton thread increased from 1.5 mm to about 2 mm. The sodium fluorescein score of mice in the CsA₃-ASO_{NFKBIZ-m} nanomicelles treatment group dropped from 10 points to about 6 points, and the phenol red cotton thread infiltration length increased from 1.5 mm to about 3.5 mm. The results of animal experiments showed that compared to the case group and the simple CsA eye drops group, the nanomicelles assembled from the conjugation of 3 CsA molecules with ASO targeting NFKBIZ had a more significant therapeutic effect on xerophthalmia in mice.

### Example 5 the Treatment of Xerophthalmia with Conjugates of Single, Double, and Triple CsA Molecules with siRNA Targeting NFKBIZ (nCsA-siRNA_{NFKBIZ}, n=1, 2, or 3)

5.1 Preparation and Characterization of Conjugates of Single, Double, and Triple CsA Molecules with siRNA_{NFKBIZ} (nCsA-siRNA_{NFKBIZ}, n=1, 2, or 3)

In this example, the siRNA targeting the NFKBIZ gene was purchased from Shanghai Sangon Biotech Co., Ltd., and n (n=1, 2, or 3) DBCO modifications were introduced at the 3′ end of the sense strand of siRNA to enable efficient coupling with the azide group modified on CsA-N₃ through a click reaction. The specific sequence information of the siRNA selected in this example was listed in detail below.

The siRNA sequences modifying 1, 2, and 3 DBCOs and targeting the human NFKBIZ gene were as follows:

DBCO-siRNA_{NFKBIZ-h}
Sense
(SEQ ID NO: 9)
5′-rGrCrCrCrGrArUrUrCrGrUrUrGrUrCrUrGrArUdTdT-
DBCO-3′;
Antisense
(SEQ ID NO: 10)
5′-rArUrCrArGrArCrArArCrGrArArUrCrGrGrGrCdTdT-3′;
2DBCO-siRNA_{NFKBIZ-h}
Sense
(SEQ ID NO: 11)
5′-rGrCrCrCrGrArUrUrCrGrUrUrGrUrCrUrGrArUdTdT/
iDBCOdT/dTdT-DBCO-3′;
Antisense
(SEQ ID NO: 12)
5′-rArUrCrArGrArCrArArCrGrArArUrCrGrGrGrCdTdT-3′;
3DBCO-siRNA_{NFKBIZ-h}
Sense
(SEQ ID NO: 13)
5′-rGrCrCrCrGrArUrUrCrGrUrUrGrUrCrUrGrArUdTdT/
iDBCOdT/dT/iDBCOdT/dTdT-DBCO-3
Antisense
(SEQ ID NO: 14)
5′-rArUrCrArGrArCrArArCrGrArArUrCrGrGrGrCdTdT-3′.

The siRNA sequences modifying 1, 2, and 3 DBCOs of the mouse NFKBIZ gene were as follows:

DBCO-siRNA_{NFKBIZ-m}
Sense
(SEQ ID NO: 15)
5′-rGrCrGrUrCrArArUrGrUrArCrCrArGrUrArUrUdTdT-
DBCO-3′
Antisense
(SEQ ID NO: 16)
5′-rArArUrArCrUrGrGrUrArCrArUrUrGrArCrGrCdCdT-3′
2DBCO-siRNA_{NFKBIZ-m}
Sense
(SEQ ID NO: 17)
5′-rGrCrGrUrCrArArUrGrUrArCrCrArGrUrArUrUdTdT/
iDBCOdT/dTdT-DBCO-3′
Antisense
(SEQ ID NO: 18)
5′-rArArUrArCrUrGrGrUrArCrArUrUrGrArCrGrCdCdT-3′
3DBCO-siRNA_{NFKBIZ-m}
Sense
(SEQ ID NO: 19)
5′-rGrCrGrUrCrArArUrGrUrArCrCrArGrUrArUrUdTdT/
iDBCOdT/dT/iDBCOdT/dTdT-DBCO-3′
Antisense
(SEQ ID NO: 20)
5′-rArArUrArCrUrGrGrUrArCrArUrUrGrArCrGrCrCdT-3′

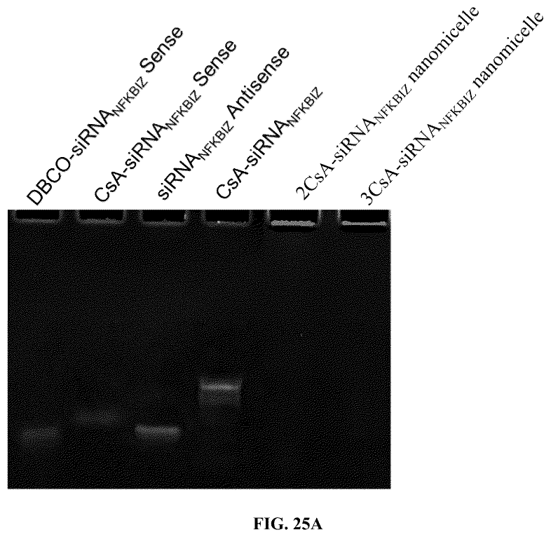

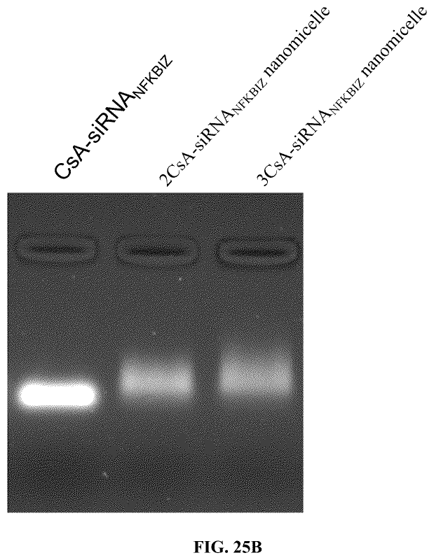

A synthesis method of the conjugate of CsA and siRNA included: 0.2 mg, 0.4 mg, and 0.6 mg of CsA-N₃ (140 nmol, 280 nmol, and 420 nmol) were dissolved in 0.14 mL of DMSO, and 5 OD of DBCO-siRNA_{NFKBIZ} Sense strand, 2DBCO-siRNA_{NFKBIZ} Sense strand, and 3DBCO-siRNA_{NFKBIZ} Sense strand (28 nmol each) were added, and then reacted by stirring at 37° C. for 24 h. 5 mL of water was added, and the excess CsA-N₃ in the reaction was removed by extraction with ethyl acetate, and then the aqueous solution was concentrated and evaporated to dryness to obtain the conjugate molecules of CsA-siRNA_{NFKBIZ} Sense strand, 2CsA-siRNA_{NFKBIZ} Sense strand, and 3CsA-siRNA_{NFKBIZ} Sense strand. The CsA-siRNA_{NFKBIZ} Sense strand and an equal amount of siRNA_{NFKBIZ} Antisense strand were mixed in 1×PBS, and the Sense strand and the Antisense strand were complementary paired to form a soluble CsA-siRNA_{NFKBIZ} conjugate double-stranded molecule. For CsA-siRNA_{NFKBIZ} Sense strand modified with two and three CsA molecules, 2CsA-siRNA_{NFKBIZ} Sense strand and 3CsA-siRNA_{NFKBIZ} Sense strand were redissolved in 50 μL DMSO and then dropped into 500 μL 1×PBS buffer for assembly. DMSO was removed by dialyzing in 1×PBS to obtain 2CsA-siRNA_{NFKBIZ} Sense strand nanomicelles and 3CsA-siRNA_{NFKBIZ} Sense strand nanomicelles, and complementary-paired with the added Antisense strand to form 2CsA-siRNA_{NFKBIZ} and 2CsA-SiRNA_{NFKBIZ} nanomicelles. The obtained product was characterized by polyacrylamide gel electrophoresis and agarose gel electrophoresis, and results were shown in FIGS. 25A-25B. The CsA-N₃ was successfully coupled to the siRNA_{NFKBIZ} Sense strand without affecting the subsequent complementary pairing and assembly with the corresponding Antisense strand.

5.2 Regulatory Effects of nCsA-siRNA_{NFKBIZ-h} (n=1, 2, 3) and its Assembled Structures on IκB-ξ Protein

nCsA-siRNA_{NFKBIZ-h} (n=1, 2, 3) nanomicelles were designed to reduce NFKBIZ mRNA levels in cells, thereby further reducing the expression at the protein level. In order to examine the expression level of IκB-(protein in HCECs and the down-regulatory effect of different drugs on it, Western blot was conducted for characterization. HCECs were inoculated in a 6-well plate at a density of 1×10⁵ cells/well and cultured until adherence. The cells were then stimulated with 100 ng/mL LPS overnight. After removing the supernatant, 1 mL of Opti-MEM medium containing siRNA_{NFKBIZ-h}+Lipo2000 complex and nCsA-siRNA_{NFKBIZ-h} (n=1, 2, 3) conjugates and nanomicelles (all containing 10 μM siRNA) were added, respectively. After incubation at 37° C. for 6 h, the cells were transferred into DMEM medium and incubated for another 48 h. The protein in the cells was extracted for Western blot analysis.

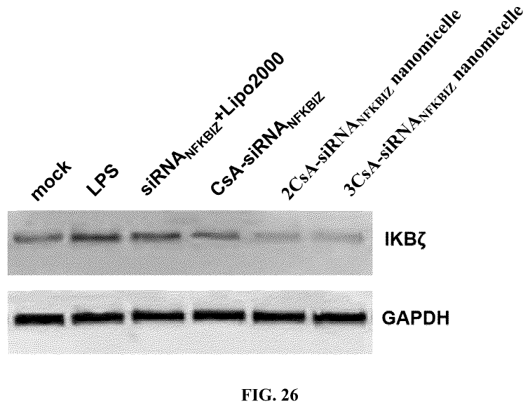

As shown in FIG. 26, the data showed that the expression level of IκB-protein in HCECs after LPS stimulation was about 1.5 times that of normal cells, the expression level of IκB-(protein after transfection of siRNA_{NFKBIZ-h} using Lipo2000 was about 1.2 times that of normal cells. After adding CsA-siRNA_{NFKBIZ-h} conjugate, 2CsA-siRNA_{NFKBIZ} nanomicelles, and 3CsA-siRNA_{NFKBIZ} nanomicelles, the expression level of IκB-C protein dropped to about 0.5 to 0.8 times that of normal cells. Therefore, nCsA-siRNA_{NFKBIZ-h} could effectively reduce the expression of IκB-(protein caused by inflammation after LPS stimulation, and the effect of reducing IκB-protein was better with the increase of the number of CsA modifications; and the effects of nCsA-siRNA_{NFKBIZ-h} (n=1, 2, 3) conjugates and nanomicelles were better than those of Lipo2000 transfected siRNA_{NFKBIZ-h} group.

5.3 Regulatory Effects of nCsA-siRNA_{NFKBIZ-h} (n=1, 2, 3) and its Assembled Structures on the NFKBIZ Gene

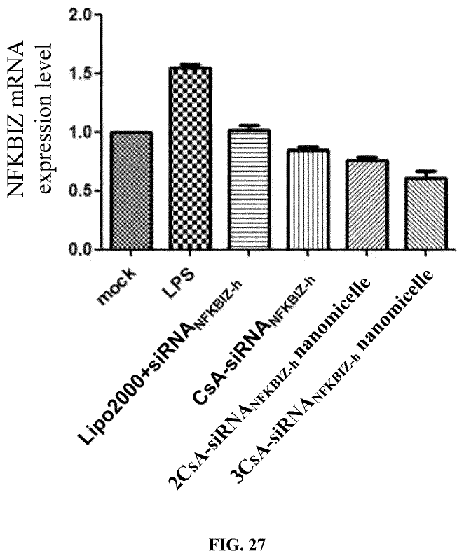

HCECs were inoculated in a 6-well plate at a density of 1×10⁵ cells/well and cultured overnight until adherence. The cells were then stimulated with 100 ng/ml LPS for 12 h. After removing the supernatant, 1 mL of Opti-MEM medium containing siRNA_{NFKBIZ-h}+Lipo2000 complex and nCsA-siRNA_{NFKBIZ-h} (n=1, 2, 3) conjugates and nanomicelles (all containing 10 μM siRNA) were added, respectively. After incubation at 37° C. for 6 h, the cells were transferred into DMEM medium and incubated for another 48 h. The cells were collected and RNA was extracted for RT-qPCR to evaluate the expression of NFKBIZ mRNA in HCECs, and experimental results were shown in FIG. 27.

Compared to the blank group (Mock group), CsA-siRNA_{NFKBIZ-h} significantly reduced the expression of NFKBIZ gene, and could reduce to the level before LPS stimulation. At the same time, the expression of NFKBIZ in HCECs after LPS stimulation increased to about 1.5 times that of normal cells. The siRNA_{NFKBIZ-h} grafted with 1, 2 and 3 CsA, with the increase of the number of CsA modifications, had an increased effect on reducing the NFKBIZ gene, and the expression of NFKBIZ in cells was reduced to about 0.5 to 0.7 times that of normal cells. This might be due to the hydrophobic effect of CsA enhancing the ability of siRNA_{NFKBIZ-h} to enter cells, and the synergistic effect of the above two had better inflammatory treatment ability.

5.4 Therapeutic Effects of nCsA-siRNA_{NFKBIZ-m} (n=1, 2, 3) and its Assembled Structures on Xerophthalmia

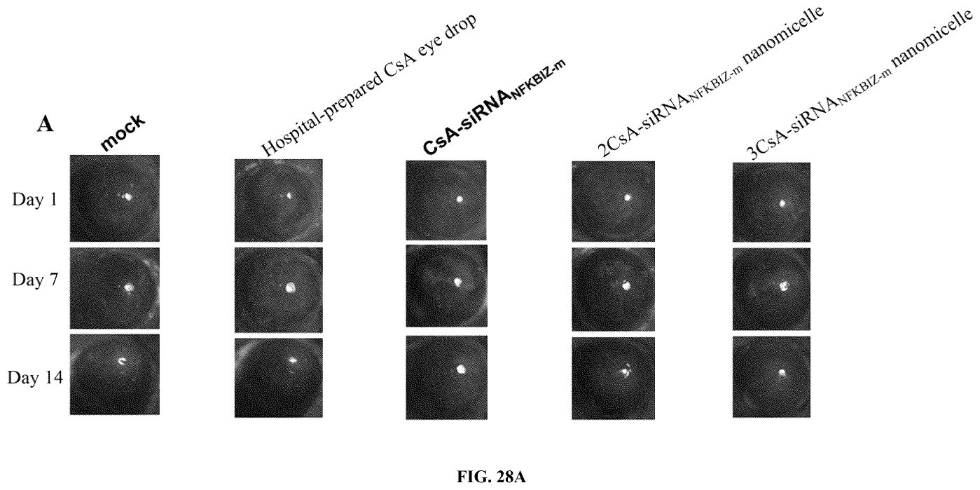

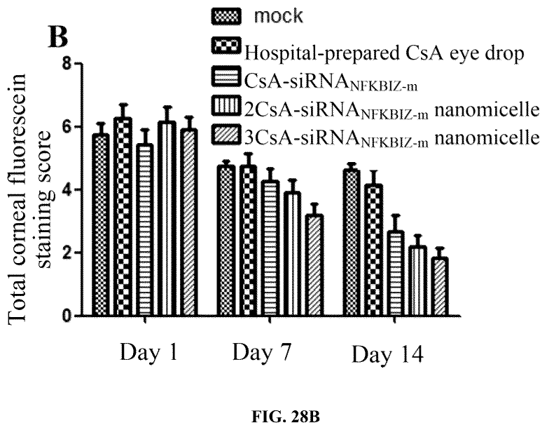

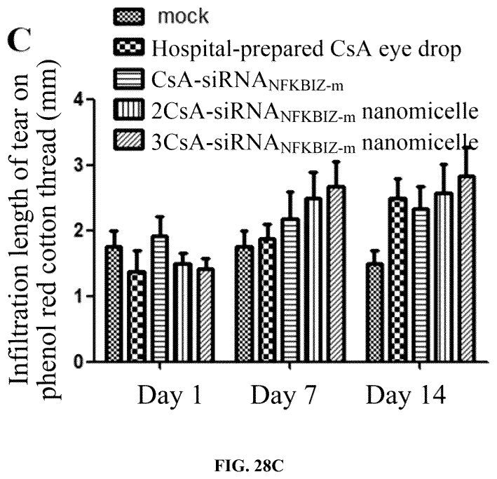

The ocular xerophthalmia model was established in 6-8 weeks old mice (C57BL/6, Suzhou Xishan Biotechnology Co., Ltd.) using 0.2% benzalkonium chloride. 5 μL of 0.2% benzalkonium chloride was dripped into each eye of the mouse, once in the morning and once in the evening every day for two weeks. After successful modeling, the hospital-prepared CsA eye drops (400 UM CsA, provided by the Eye, Ear, Nose and Throat Hospital Affiliated to Fudan University School of Medicine, Shanghai) and CsA-siRNA_{NFKBIZ-m} (400 UM CsA), 2CsA-siRNA_{NFKBIZ-m} nanomicelles (400 UM CsA), 3CsA-siRNA_{NFKBIZ-m} nanomicelles (400 UM CsA) were used for eye drops treatment, 5 μL per eye, once in the morning and once in the evening. Sodium fluorescein staining and phenol red cotton thread tear secretion test were conducted on days 0, 7, and 14 of treatment to evaluate the therapeutic effect. The experimental results were shown in FIGS. 28A-28C.

The results of sodium fluorescein staining showed that the eye score of mice treated with hospital-prepared CsA eye drops dropped from 6 points to about 4.5 points, and the infiltration length of tear on phenol red cotton thread increased from 1.5 mm to about 2.5 mm; the eye score of mice treated with CsA-siRNA_{NFKBIZ} dropped from 6 points to about 3 points, and the infiltration length of tear on phenol red cotton thread increased from 2 mm to about 2.5 mm; the eye score of mice treated with 2CsA-siRNA_{NFKBIZ} nanomicelles dropped from 6 points to about 2 points, and the infiltration length of tear on phenol red cotton thread increased from 1.5 mm to about 2.5 mm; the eye score of mice treated with 3CsA-siRNA_{NFKBIZ} nanomicelles dropped from 6 points to about 2 points, and the infiltration length of tear on phenol red cotton thread increased from 1.5 mm to about 2.5 mm. From the results of animal experiments, it could be seen that compared to the case group and the simple CsA eye drops group, the CsA-siRNA_{NFKBIZ-m} conjugate, 2CsA-siRNA_{NFKBIZ-m} nanomicelles, and 3CsA-siRNA_{NFKBIZ-m} nanomicelles had more significant therapeutic effects on xerophthalmia in mice. This is due to two factors: first, the synergistic treatment of small molecule drug and nucleic acid drug could better achieve inhibiting mouse eye cell inflammation; and second, the hydrophobic CsA endowed siRNA better cell-entry ability.

### Example 6 the Treatment of Xerophthalmia with Conjugates of Multiple CsA Molecules Grafted with NFKBIZ ASO and their Self-Assembled Nanomicelles

### 6.1 Synthesis and Characterization of Carbonylethyl Bromo-CsA (CsA-Br)

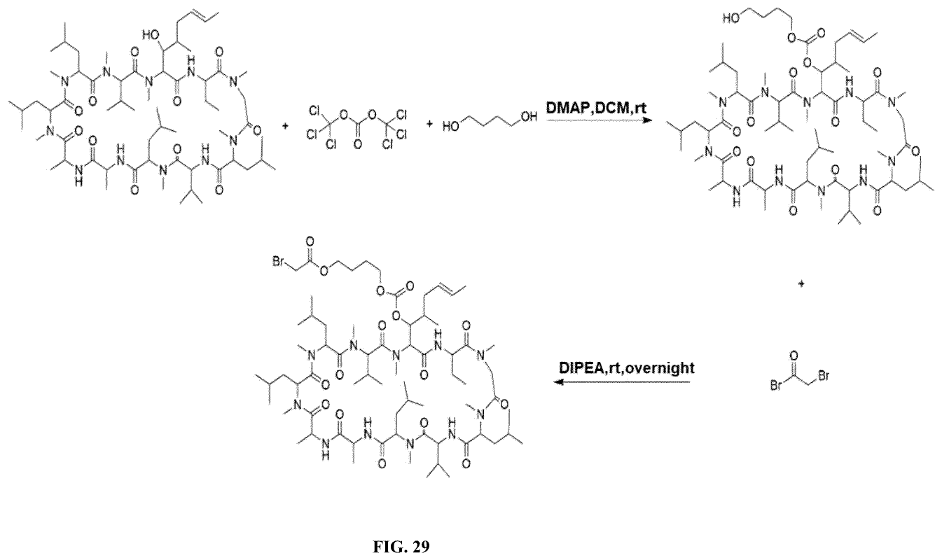

As shown in FIG. 29, CsA (100 mg) and triphosgene (9 mg) were placed in a flask, and the air was evacuated three times and then the mixture was dissolved in 16 mL of DCM under nitrogen protection. DMAP (31 mg) was dissolved in 1 mL of DCM and added dropwise to a resulting reaction solution. The mixture was reacted at room temperature for 30 min, and the solution turned milky white. Butanediol (72 mg) was dissolved in 1 mL of DCM and added dropwise to the reaction solution. The mixture was reacted at room temperature overnight, and the solution turned from milky white to colorless and transparent. After the reaction, the sample was washed three times with dilute hydrochloric acid solution and 1 time with saturated sodium chloride solution according to the amount of DMAP, and dried with anhydrous sodium sulfate powder for more than 2 h. The organic phase was concentrated by vacuum distillation and then separated and purified by silica gel column chromatography, with an eluent being a mixed solution of petroleum ether/ethyl acetate, to obtain a white solid, namely butanediol-modified CsA molecule.

CsA-butanediol (90 mg) and DIPEA (8 mg) were placed in a flask. After evacuating the air three times, 16 mL of DCM was added under nitrogen protection, and bromoacetyl bromide (16 mg) was dissolved in 1 mL of DCM and added dropwise to a resulting reaction solution, and reaction was allowed to react at room temperature overnight. After the reaction was completed, the product was washed three times with a dilute hydrochloric acid solution according to the amount of DIPEA, washed 1 time with a saturated sodium chloride solution, dried with anhydrous sodium sulfate powder for more than 2 h, and the organic phase was concentrated by vacuum distillation and then separated and purified by silica gel column chromatography, with an eluent being a mixed solution of petroleum ether/ethyl acetate, to finally obtain a yellow solid, namely the CsA-Br. ¹H NMR (700 MHZ, Chloroform-d) & 7.99 (d, J=9.7 Hz, 1H), 7.64 (d, J=7.5 Hz, 1H), 7.47 (d, J=8.3 Hz, 1H), 7.41 (s, 1H), 7.28 (s, 1H), 7.16 (d, J=7.9 Hz, 2H), 7.11 (s, 1H), 5.70 (d, J=6.6 Hz, 1H), 5.48 (d, J=6.3 Hz, 1H), 5.36-5.32 (m, 3H), 5.12 (d, J=10.9 Hz, 1H), 5.08-5.02 (m, 3H), 4.97 (dd, J=9.9, 5.9 Hz, 1H), 4.85-4.81 (m, 2H), 4.72 (d, J=13.9 Hz, 1H), 4.65 (dd, J=9.9, 8.4 Hz, 2H), 4.52 (t, J=7.3 Hz, 2H), 3.80 (t, J=6.5 Hz, 2H), 3.51 (s, 3H), 3.40 (s, 3H), 3.26 (s, 3H), 3.20 (s, 1H), 3.18 (s, 1H), 3.11 (d, J=1.6 Hz, 5H), 2.96 (s, 1H), 2.89 (s, 1H), 2.69 (d, J=5.8 Hz, 6H), 2.41 (dd, J=12.1, 7.7 Hz, 2H), 2.13 (d, J=11.1 Hz, 2H), 2.08-2.05 (m, 2H), 1.99 (s, 1H), 1.77 (s, 2H), 1.73-1.69 (m, 2H), 1.63 (d, J=7.4 Hz, 4H), 1.58 (s, 6H), 1.47 (d, J=6.5 Hz, 1H), 1.43 (s, 1H), 1.35 (d, J=7.3 Hz, 3H), 1.25 (d, J=6.7 Hz, 6H), 1.07 (d, J=6.5 Hz, 2H), 1.02 (dd, J=14.8, 6.6 Hz, 7H), 0.97-0.93 (m, 8H), 0.89-0.84 (m, 13H), 0.83 (d, J=6.6 Hz, 3H), 0.71 (d, J=6.1 Hz, 3H).

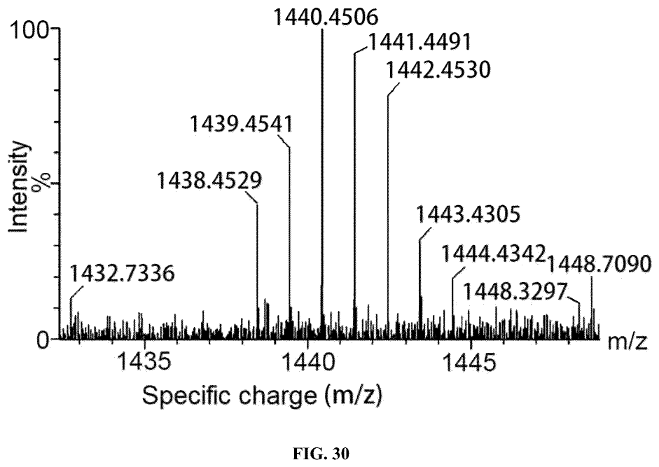

CsA-Br had theoretical m/z values of 1,437.8098 and 1,439.8077 due to the existence of isotopic ion peaks of bromine element. The m/z values measured by HPLC/quadrupole rod flight mass spectrometry were 1,438.4549 and 1,440.4506, which were attributed to the [M+H]⁺ peak of CsA-Br, confirming that the target product was successfully synthesized, as shown in FIG. 30.

6.2 Preparation and Characterization of 20CSA-ASO_{NFKBIZ} conjugates and 20CsA-ASO_{NFKBIZ} Nanomicelles

In this example, the ASO targeting the NFKBIZ gene was purchased from Shanghai Sangon Biotech Co., Ltd. The phosphate backbone of the ASO was modified with thio (PS) to enable ASO to undergo substitution with a bromocarbonylethyl group modified on the CsA-Br for efficient coupling. The sequence was as follows:

the sequence of the ASO targeting human NFKBIZ gene (20PS-ASO_{NFKBIZ-h}) was:

5′-ATCAGACAACGAATCGGGCTTTTTT*T*T*T*T*T*T*T*T*T*T*T
*T*T*T*T*T*T*T*T*T-3′

(* indicated a phosphorothioate group modification site, and indicated that the two nucleotide units before and after were ligated by a phosphorothioate group; in the following examples, unless otherwise specified, phosphorothioate modifications in the nucleic acid sequences were indicated by *, and the underlined TTTTT was a multiple T base spacer sequence connecting the 5′ end ASO and 3′ end for the drug modification sequence; the underlined parts of the sequences listed in the following examples all represented the spacer sequence ligating the functional small nucleic acid sequence and the drug modification sequence, which would not be repeated one by one)(SEQ ID NO: 21);

the sequence of the ASO targeting mouse NFKBIZ gene (20PS-ASO_{NFKBIZ-m}) was:

(SEQ ID NO: 22)
5′-AATACTGGTACATTGACGCCTTTTTT*T*T*T*T*
T*T*T*T*T*T*T*T*T*T*T*T*T*T*T*T-3′

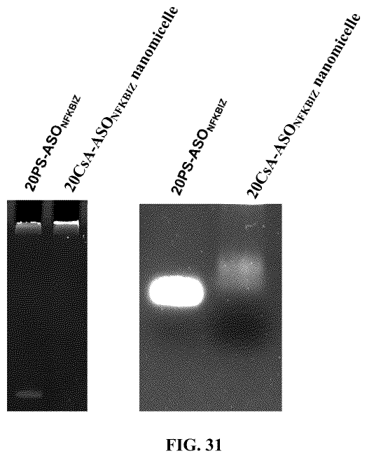

The synthesis method was as follows: 7.5 mg of CsA-Br (5 μmol) was dissolved in 0.063 mL of DMSO, and 5 OD of 20PS-ASO_{NFKBIZ} (12.5 nmol) was added, and the mixture was reacted by shaking at 50° C. for 24 h. 5 mL of water was added, and the excess CsA-Br in the reaction was removed by extraction with ethyl acetate, and then the aqueous solution was concentrated and evaporated to dryness to obtain the 20CsA-ASO_{NFKBIZ} conjugate molecule. The 20CsA-ASO_{NFKBIZ} conjugate was dissolved in 50 μL of DMSO, added dropwise to 0.5 mL of PBS, and stirred for 30 min. Then, the mixture was placed in a dialysis bag and dialyzed in PBS overnight to remove DMSO, thereby obtaining the 20CsA-ASO_{NFKBIZ} nanomicelle structure. The successful grafting coupling and the successful assembly were verified by 10% denatured polyacrylamide gel electrophoresis and 1% agarose gel electrophoresis. As shown in FIG. 31, the CsA-Br was successfully grafted onto 20PS-ASO_{NFKBIZ} to form a 20PS-ASO_{NFKBIZ} nanomicelle assembly with relatively uniform size.

### 6.3 Knockdown Effect of NFKBIZ Gene by 20CsA-ASO_{NFKBIZ-h} Nanomicelles

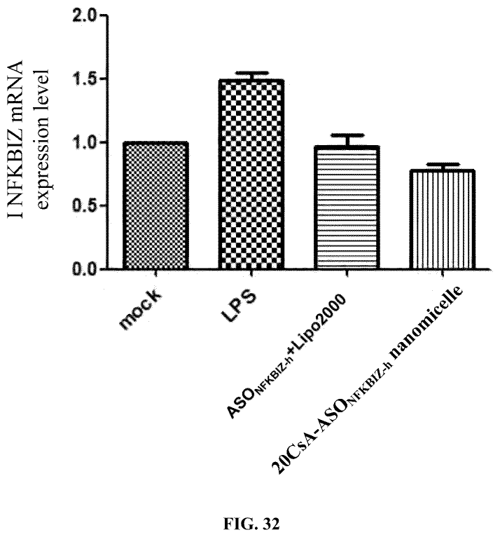

HCECs were inoculated in a 6-well plate at a density of 1×10⁵ cells/well and cultured overnight until adherence. The cells were then stimulated with 100 ng/ml LPS for 12 h. After removing the supernatant, 1 mL of Opti-MEM medium containing ASO_{NFKBIZ-h}+Lipofectamine2000 (Lipo2000) complex and 20CsA-ASO_{NFKBIZ-h} nanomicelles (all containing 10 UM ASO) were added, respectively. After incubation at 37° C. for 6 h, the cells were transferred into DMEM medium and incubated for another 48 h. The cells were collected and RNA was extracted for RT-qPCR to evaluate the expression of NFKBIZ mRNA in HCECs, and experimental results were shown in FIG. 32.

After LPS stimulation, the expression of NFKBIZ mRNA in HCECs increased to about 1.5 times that of normal cells, and ASO_{NFKBIZ-h} after Lipo2000 transfection could downregulate it to the level of normal cells. 20CsA-ASO_{NFKBIZ-h} nanomicelles down-regulated the NFKBIZ gene to about 0.75 times that of normal cells. Therefore, compared to a single ASO_{NFKBIZ-h}, 20CsA-ASO_{NFKBIZ-h} nanomicelles had a better ability to down-regulate the expression of the NFKBIZ gene.

### 6.4 Therapeutic Effect of 20CsA-ASO_{NFKBIZ-m} Nanomicelles on Xerophthalmia

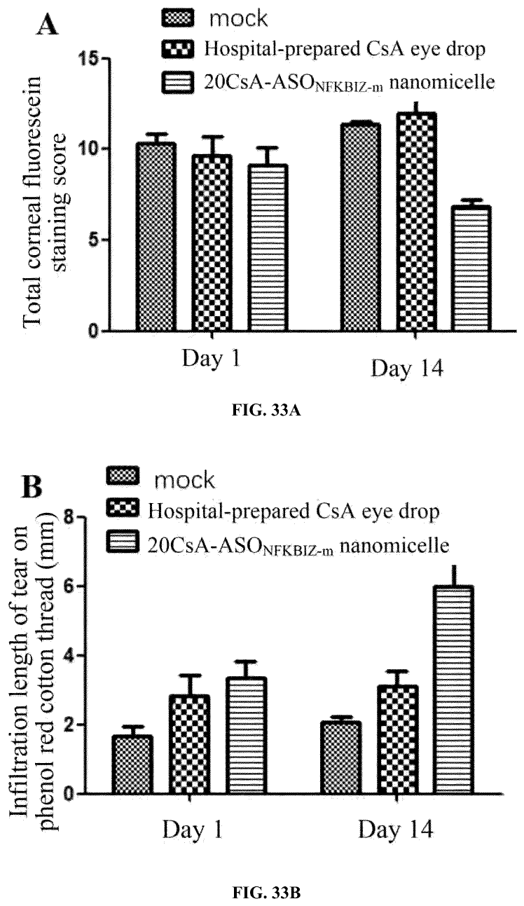

The ocular xerophthalmia model was established in 6-8 weeks old mice (C57BL/6, Suzhou Xishan Biotechnology Co., Ltd.) with 0.2% benzalkonium chloride. 5 μL of 0.2% benzalkonium chloride was dripped into each eye of the mice, once in the morning and once in the evening every day, for two consecutive weeks. After successful modeling, the hospital-prepared CsA eye drops (400 μM CsA, provided by the Eye, Ear, Nose and Throat Hospital Affiliated to Fudan University School of Medicine, Shanghai) and 20CsA-ASO_{NFKBIZ-m} nanomicelles (20 μM, 400 UM CsA) were used for eye drops treatment, 5 μL per eye, once in the morning and once in the evening. Sodium fluorescein staining and phenol red cotton thread tear secretion test were conducted on days 0 and 14 of treatment to evaluate the therapeutic effect. The experimental results were shown in FIGS. 33A-33B.

The results of sodium fluorescein staining showed that the eye score of mice treated with hospital-prepared CsA eye drops increased from 9 points to about 11 points, with no significant improvement, and the infiltration length of tear on phenol red cotton thread remained at about 2.5 mm without significant changes; after treatment with 20CsA-ASO_{NFKBIZ-m} nanomicelles, the sodium fluorescein score of mice eyes decreased from 9 points to about 7.5 points, and the infiltration length of tear on phenol red cotton thread increased from 3 mm to about 5.5 mm. Compared to hospital-prepared CsA eye drops, 20CsA-ASO_{NFKBIZ-m} nanomicelles showed significant therapeutic effect on xerophthalmia in mice within two weeks.

### Example 7 the Treatment of Xerophthalmia with Covalent Conjugates of CsA Molecule with siRNAs Targeting IL-17, IL-1β, IL-23, and TNF-α and their Self-Assembled Nanomicelles

7.1 Preparation of Conjugates of CsA and siRNAs with Different Targets and their Nanomicelles

In this example, the siRNA targeting genes of different inflammatory factors was purchased from Shanghai Sangon Biotech Co., Ltd., and 3 DBCO modifications were introduced at the 3′ end of the sense strand of siRNA to enable efficient coupling with the azide group modified on CsA-N₃ through a click reaction, and the sequences were as follows:

the siRNA sequences targeting different human inflammatory factor genes were:

IL-17 human (3DBCO-siRNA_{IL-17-h})
Sense
(SEQ ID NO: 23)
5′-rCrUrCrUrArArUrGrArGrUrUrUrArGrUrCrCrGrAdTd
T/iDBCOdT/dT/iDBCOdT/dTdT-DBCO-3′
Antisense
(SEQ ID NO: 24)
5′-rUrCrGrGrArCrUrArArArCrUrCrArUrUrArGrArGdT
dT-3′
IL-1β human (3DBCO-siRNA_{IL-1β-h})
Sense
(SEQ ID NO: 25)
5′-rArGrGrCrUrGrArUrCrUrGrUrUrGrCrCrGrUrAdTdT/
iDBCOdT/dT/iDBCOdT/dTdT-DBCO-3′
Antisense
(SEQ ID NO: 26)
5′-rUrArCrGrGrCrArArCrArGrArUrCrArGrCrCrUdTdG-3′
IL-23 human (3DBCO-siRNA_{IL-23-h})
Sense
(SEQ ID NO: 27)
5′-rCrArGrCrArArCrCrCrUrGrArGrUrCrCrCrUrAdTdT/
iDBCOdT/dT/iDBCOdT/dTdT-DBCO-3′
Antisense
(SEQ ID NO: 28)
5′-rUrArGrGrGrArCrUrCrArGrGrGrUrUrGrCrUrGdTdT-3′
TNF-α human (3DBCO-siRNA_{TNF-α-h})
Sense
(SEQ ID NO: 29)
5′-rGrArCrArArCrCrArArCrUrArGrUrGrGrUrGrCdTdT/
iDBCOdT/dT/iDBCOdT/dTdT-DBCO-3′
Antisense
(SEO ID NO: 30)
5′-rGrCrArCrCrArCrUrArGrUrUrGrGrUrUrGrUrCdTdT-3′

the siRNA sequences targeting different mouse inflammatory factor genes were:

IL-17 mouse (3DBCO-siRNA_{IL-17-m})
(SEQ ID NO: 31)
rArArGrArGrArUrCrCrUrGrGrUrCrCrUrGrArAdTdT/
iDBCOdT/dT/iDBCOdT/dTdT-DBCO-3′
Antisense
(SEQ ID NO: 32)
5′-rUrUrCrArGrGrArCrCrArGrGrArUrCrUrCrUrUdGdC-3′
IL-1β mouse (3DBCO-siRNA_{IL-1β-m})
Sense
(SEQ ID NO: 33)
5′-rGrGrArArGrGrCrArGrUrGrUrCrArCrUrCrArUdTdT/
iDBCOdT/dT/iDBCOdT/dTdT-DBCO-3′
Antisense
(SEQ ID NO: 34)
5′-rArUrGrArGrUrGrArCrArCrUrGrCrCrUrUrCrCdTdT-3′
IL-23 mouse (3DBCO-siRNA_{IL-23-m})
Sense
(SEQ ID NO: 35)
5′-
rArCrArArCrCrArUrCrArCrCrArCrArCrUrGrGrArUrArCr
GrGdTdT/iDBCOdT/dT/iDBCOdT/dTdT-DBCO-3′
Antisense
(SEQ ID NO: 36)
5′-rCrCrGrUrArUrCrCrArGrUrGrUrGrGrUrGrArUr
GrGrUrUrGrUdTdT-3′
TNF-α mouse (3DBCO-siRNA_{TNF-α-m})
Sense
(SEQ ID NO: 37)
5′-rGrUrCrUrCrArGrCrCrUrCrUrUrCrUrCrArUrUrCrCr
UdTdT/iDBCOdT/dT/iDBCOdT/dTdT-DBCO-3′
Antisense
(SEQ ID NO: 38)
5′-rArGrGrArArUrGrArGrArArGrArGrGrCrUrGrArGrAr
CdTdT-3′

The synthesis method of 3CsA-siRNA conjugates for different targets was as follows: 30 times the molar equivalent of CsA-N₃ was dissolved in DMSO, 5 OD of 3DBCO-siRNA Sense strands with different targets were added to make the reaction concentration 200 μM, and then placed at 37° C. for shaking reaction for 24 h. After adding water, the excess CsA-N₃ in the reaction was removed by extraction with ethyl acetate, and the mixture was concentrated and evaporated to dryness to obtain conjugate molecules of 3CsA-siRNA_{IL-17} Sense strand, 3CsA-siRNA_{IL-1β} Sense strand, 3CsA-siRNA_{IL-23} Sense strand, and 3CsA-siRNA_{TNF-α} Sense strand. The conjugate molecules were redissolved with 50 μL DMSO solution and then dropped into 500 μL PBS for assembly to form 3CsA-siRNA_{IL-17} Sense strand nanomicelles, 3CsA-siRNA_{IL-1β} Sense strand nanomicelles, 3CsA-siRNA_{IL-23} Sense strand nanomicelles, and 3CsA-siRNA_{TNF-α} Sense strand nanomicelles, and then dialyzed in PBS to remove DMSO. The nanomicelles were complementary-paired with the Antisense strand to obtain 3CsA-siRNA_{IL-17} nanomicelles, 3CsA-siRNA_{IL-1β} nanomicelles, 3CsA-siRNA_{IL-23} nanomicelles, and 3CsA-siRNA_{TNF-α} nanomicelle samples dispersed in PBS solution.

### 7.2 Regulatory Effects of 3CsA-siRNA Nanomicelles for Different Targets on Target Gene

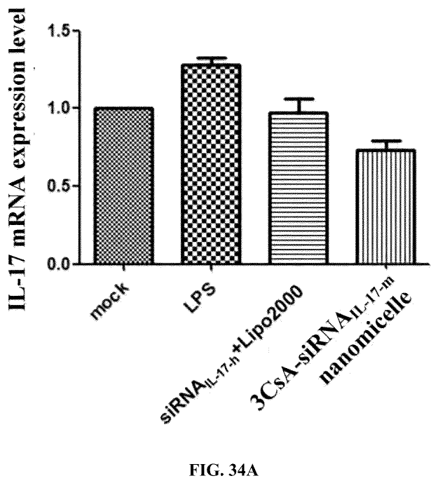

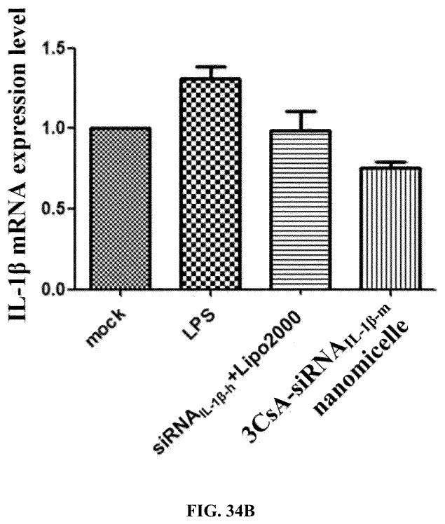

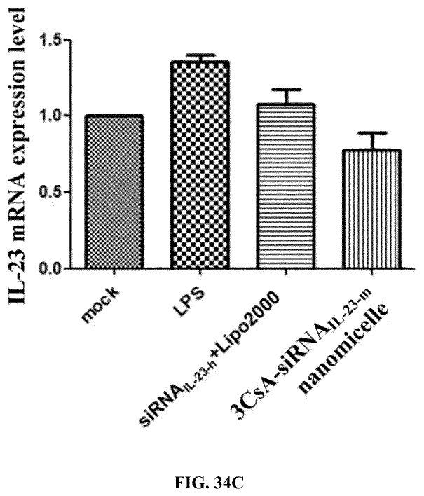

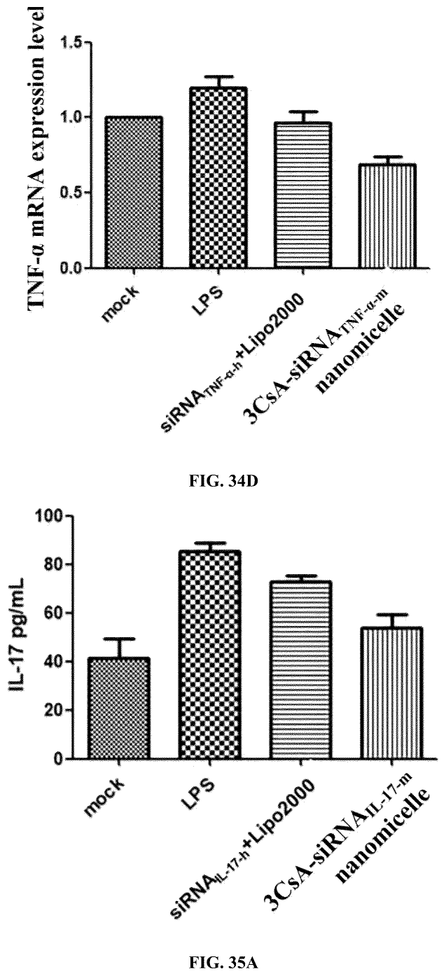

HCECs were inoculated in a 6-well plate at a density of 1×10⁵ cells/well and cultured overnight until adherence. The cells were then stimulated with 100 ng/ml LPS for 12 h. After removing the supernatant, 1 mL of Opti-MEM medium containing 3CsA-siRNA_{IL-17-h} nanomicelles, 3CSA-siRNA_{IL-1}β-h nanomicelles, 3CsA-siRNA_{IL-23-h} nanomicelles, and 3CsA-siRNA_{TNF-α-h} nanomicelles (all containing 10 μM siRNA) were added, respectively, while control group was 1 mL of Opti-MEM medium containing different target siRNA and Lipo2000 complex (the siRNA concentration was 10 μM). After incubation at 37° C. for 6 h, the cells were transferred into DMEM medium and incubated for another 48 h. The cells were collected and RNA was extracted for RT-qPCR to evaluate the expression of mRNA of different targets in HCECs. The experimental results were shown in FIGS. 34A-34D.

After LPS stimulation, the mRNA expression levels of different inflammatory factors were upregulated, which was about 1.2 to 1.4 times the expression level of each inflammatory mRNA in normal cells. After adding Lipo2000 to transfect siRNAs of different targets, the expression of different inflammatory factors was reduced to 0.8-1.1 times the normal cell level; after adding nanomicelle materials of different targets, the expression of different inflammatory factors was reduced to about 0.75 times the normal cell level. The data showed that 3CsA-siRNA nanomicelle materials with different targets had a desirable effect of downregulating inflammatory factor genes.

### 7.3 Inhibitory Effect of 3CsA-siRNA Nanomicelles Targeting Different Targets on the Expression of Related Inflammatory Factors

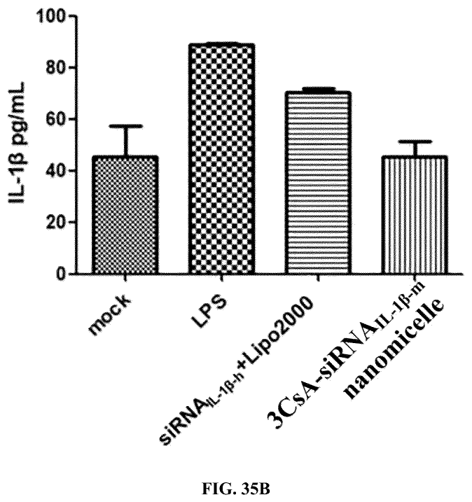

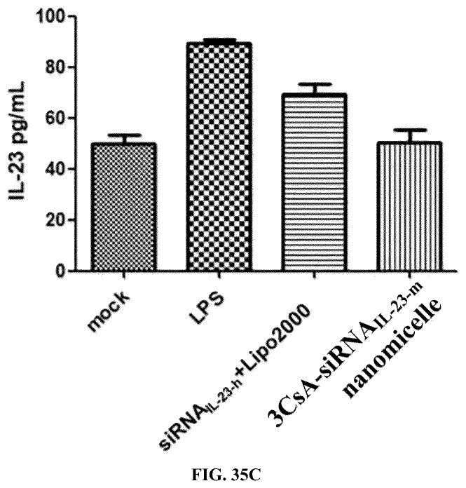

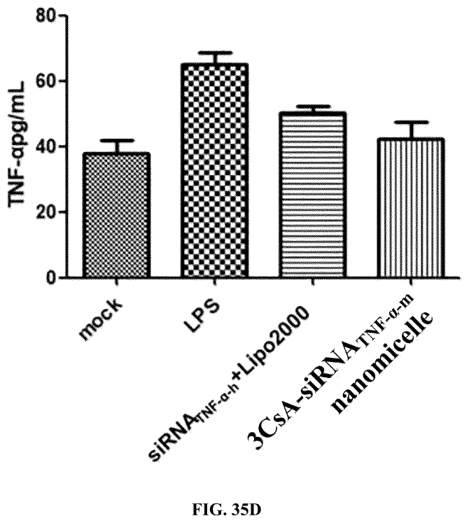

HCECs were inoculated in a 12-well plate at a density of 5×10⁴ cells/well and cultured overnight until adherence. The cells were then stimulated with 100 ng/ml LPS for 12 h. After removing the supernatant, 1 mL of Opti-MEM medium containing 3CsA-siRNA_{IL-17-h} nanomicelles, 3CSA-siRNA_{IL-1β-h} nanomicelles, 3CsA-siRNA_{IL-23-h} nanomicelles, and 3CsA-siRNA_{TNF-α-h} nanomicelles (all containing 10 μM siRNA) were added, respectively, while control group was 1 mL of Opti-MEM medium containing different target siRNA and Lipo2000 complex (the siRNA concentration was 10 μM). After incubation at 37° C. for 6 h, the cells were transferred into DMEM medium and incubated for another 48 h. The cells were collected and RNA was extracted for ELISA to detect the expression of different inflammatory factors in HCECs, and experimental results were shown in FIGS. 35A-35D.

After LPS stimulation, the expression of various inflammatory factors in HCECs increased significantly, with the increase range being about 1.5 to 2.5 times that of normal cells; after adding different target siRNAs transfected with Lipo2000, the expression of inflammatory factors decreased; the expression of inflammatory factors in HCECs treated with various nanomicelle materials decreased the most and returned to the expression level of normal cells. This was because the drug-nucleic acid conjugate assembled into nanomicelles had better cellular entry ability; and secondly, due to the synergistic effect of CsA and siRNA, a better inflammation inhibition effect was achieved.

### 7.4 Therapeutic Effects of 3CsA-siRNA Nanomicelles Targeting Different Targets on Xerophthalmia

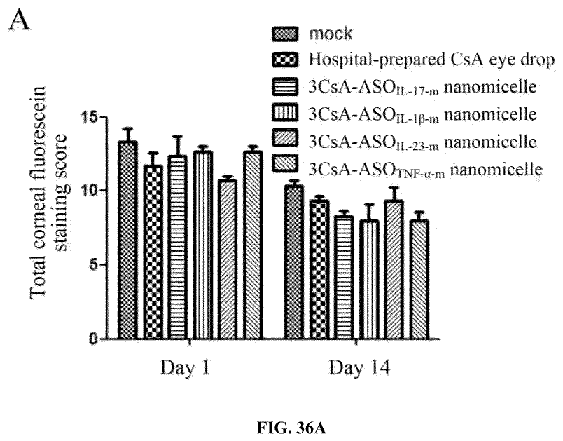

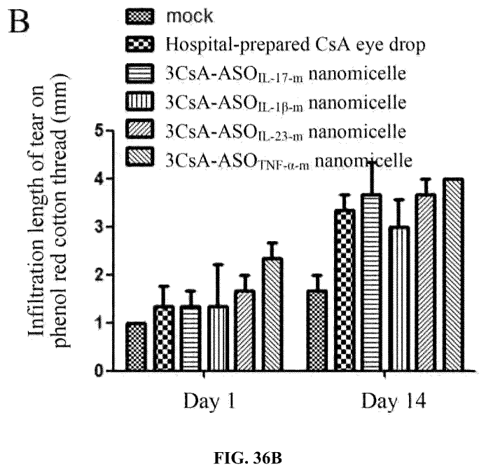

The ocular xerophthalmia model was established in 6-8 weeks old mice (C57BL/6, Suzhou Xishan Biotechnology Co., Ltd.) using 0.2% benzalkonium chloride. 5 μL of 0.2% benzalkonium chloride was dripped into each eye of the mouse, once in the morning and once in the evening every day for two weeks. After successful modeling, the hospital-prepared CsA eye drops (400 UM CsA, provided by the Eye, Ear, Nose and Throat Hospital Affiliated to Fudan University School of Medicine, Shanghai) and 3CsA-siRNA_{IL-17-m} nanomicelles, 3CsA-siRNA_{IL-1β-m} nanomicelles, 3CsA-siRNA_{IL-23-m} nanomicelles, and 3CsA-siRNA_{TNF-α-m} nanomicelles (the CsA concentration in each group of materials was 400 μM) were used for eye drops treatment, 5 μL per eye, once in the morning and once in the evening. Sodium fluorescein staining and phenol red cotton thread tear secretion test were conducted on days 0 and 14 of treatment to evaluate the therapeutic effect. The experimental results were shown in FIGS. 36A-36B.

The results of sodium fluorescein staining showed that the eye score of mice in the untreated control group dropped from 13 points to about 10 points, and the infiltration length of tear on phenol red cotton thread increased from 1 mm to about 1.3 mm, with no significant improvement; the eye score of mice treated with CsA eye drops dropped from 12 points to about 9 points, and the infiltration length of tear on phenol red cotton thread increased from 1.5 mm to about 3 mm; the sodium fluorescein staining score of mice in the siRNA nanomicelle treatment group with different targets dropped from about 13 points to about 8 points, and the infiltration length of tear on phenol red cotton thread increased from 1.5 mm to about 4 mm. The data showed that 3CsA-siRNA conjugate nanomicelles targeting different inflammatory factor targets had a certain therapeutic effect on xerophthalmia in mice within two weeks.

### Example 8 the Treatment of Xerophthalmia with Covalent Conjugates of CsA Molecule with siRNAs Targeting NLRP3, JAK1, and PDE4 and Self-Assembled Nanomicelles Thereof

8.1 Preparation of Conjugates of CsA and siRNAs with Different Targets and their Self-Assembled Nanomicelles

In this example, the siRNA targeting genes of different inflammatory genes in the cell were purchased from Shanghai Sangon Biotech Co., Ltd., and 3 DBCO modifications were introduced at the 3′ end of the sense strand of siRNA to enable efficient coupling with the azide group modified on CsA-N₃ through a click reaction, and the sequences were as follows:

the siRNA sequences targeting different human inflammation-related genes were:

NLRP3 human (3DBCO-siRNA_{NLRP3-h})
Sense
(SEQ ID NO: 39)
5′-rGrUrGrGrArCrUrUrGrArArGrArArArUrUrUrA
dTdT/iDBCOdT/dT/iDBCOdT/dTdT-DBCO-3′
Antisense
(SEQ ID NO: 40)
5′-rUrArArArUrUrUrCrUrUrCrArArGrUrCrCrArCdTdT-3′
JAK1 human (3DBCO-siRNA_{JAK1-h})
Sense
(SEQ ID NO: 41)
5′-rGrGrArUrUrArCrArArGrGrArUrGrArCrGrArArGr
GdTdT/iDBCOdT/dT/iDBCOdT/dTdT-DBCO-3′
Antisense
(SEQ ID NO: 42)
5′-rUrUrCrGrUrCrArUrCrCrUrUrGrUrArArUrCrCr
ArUdTdT-3′
PDE4 human (3DBCO-siRNA_{PDE4-h})
Sense
(SEQ ID NO: 43)
5′-rGrArGrUrCrGrGrUrCrUrGrGrArArArUrCrArAdTd
T/iDBCOdT/dT/iDBCOdT/dTdT-DBCO-3′
Antisense
(SEQ ID NO: 44)
5′-rUrUrGrArUrUrUrCrCrArGrArCrCrGrArCrUrCdTdT-3′

the siRNA sequences targeting different mouse inflammation-related genes were:

NLRP3 mouse (3DBCO-siRNA_{NLRP3-m})
Sense
(SEQ ID NO: 45)
5′-rGrGrUrGrArArArUrGrUrArCrUrUrArArArUrCdTdT/
iDBCOdT/dT/iDBCOdT/dTdT-DBCO-3′
Antisense
(SEQ ID NO: 46)
5′-rGrArUrUrUrArArGrUrArCrArUrUrUrCrArCrCdTdT-3′
JAK1 mouse (3DBCO-siRNA_{JAK1-m})
Sense
(SEQ ID NO: 47)
5′-rCrUrGrUrArUrGrGrCrGrArCrArUrUrCrUrCrCrArAdT
dT/iDBCOdT/dT/iDBCOdT/dTdT-DBCO-3′
Antisense
(SEQ ID NO: 48)
5′-rUrUrGrGrArGrArArUrGrUrCrGrCrCrArUrArCrArGdTdT-3′
PDE4 mouse (3DBCO-siRNA_{PDE4-m})
Sense
(SEQ ID NO: 49)
5′-rArUrGrArGrCrGrUrGrUrArGrArGrArGrGrArCrArAdT
dT/iDBCOdT/dT/iDBCOdT/dTdT-DBCO-3′
Antisense
(SEQ ID NO: 50)
5′-rUrUrGrUrCrCrUrCrUrCrUrArCrArCrGrCrUrCrAr
UdTdT-3′

The synthesis method of 3CsA-siRNA conjugates for different targets was as follows: 30 times the molar equivalent of CsA-N₃ was dissolved in DMSO, 5 OD of 3DBCO-siRNA Sense strands with different targets were added to make the reaction concentration 200 μM, and then placed at 37° C. for shaking reaction for 24 h. The water was added, and the excess CsA-N₃ in the reaction was removed by extraction with ethyl acetate, and then the aqueous solution was concentrated and evaporated to dryness to obtain the conjugate molecules of 3CsA-siRNA_{NLRP3} Sense strand, 3CsA-siRNA_{JAK1} Sense strand, and 3CsA-siRNA_{PDE4} Sense strand. The conjugate molecules were redissolved with 50 μL DMSO solution and then dropped into 500 μL PBS for assembly to form 3CsA-siRNA_{NLRP3} Sense strand nanomicelles, 3CsA-siRNA_{JAK1} Sense strand nanomicelles, and 3CsA-siRNA_{PDE4} Sense strand nanomicelles, and then dialyzed in PBS to remove DMSO. The nanomicelles were complementary-paired with the Antisense strand to obtain samples of 3CsA-siRNA_{NLRP3} nanomicelles, 3CsA-siRNA_{JAK1} nanomicelles, and 3CsA-siRNA_{PDE4} nanomicelles dispersed in PBS solution.

### 8.2 Regulatory Effects of 3CsA-siRNA Conjugate Nanomicelles for Different Targets on Target Gene

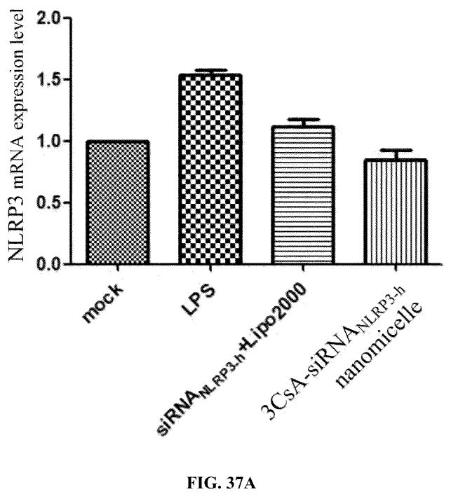

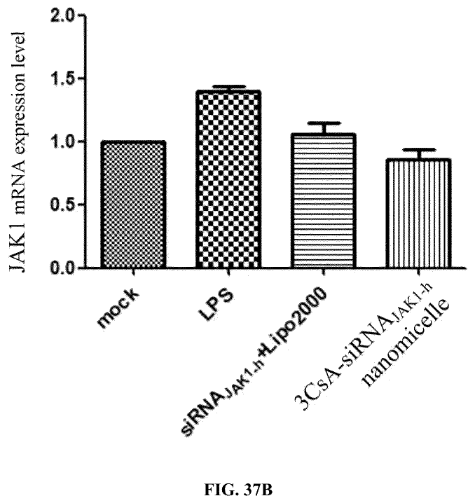

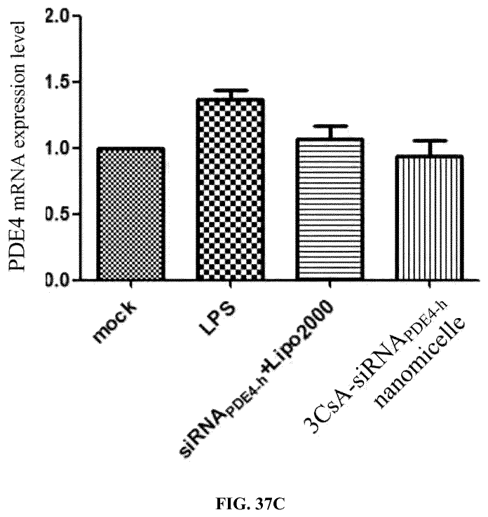

HCECs were inoculated in a 6-well plate at a density of 1×10⁵ cells/well and cultured overnight until adherence. The cells were then stimulated with 100 ng/ml LPS for 12 h. After removing the supernatant, 1 mL of Opti-MEM medium containing 3CsA-siRNA_{NLRP3-h} nanomicelles, 3CsA-siRNA_{JAK1-h} nanomicelles, and 3CsA-siRNA_{PDE4-h} nanomicelles (all containing 10 μM siRNA) were added, respectively, while control group was 1 mL of Opti-MEM medium containing different target siRNA and Lipo2000 complex (the siRNA concentration was 10 μM). After incubation at 37° C. for 6 h, the cells were transferred into DMEM medium and incubated for another 48 h. The cells were collected and RNA was extracted for RT-qPCR to evaluate the expression of mRNA of different targets in HCECs. The experimental results were shown in FIGS. 37A-37C.

After LPS stimulation, the mRNA expression levels of different intracellular inflammatory targets were significantly upregulated, which was about 1.4 times the expression level of each inflammatory mRNA in normal cells. After Lipo2000 transfecting siRNAs with different targets, the expression of inflammatory target genes in different cells was reduced to about 1.1 times the level of normal cells; after adding nanomicelle materials with different targets, the expression of inflammatory target genes in different cells was reduced to about 0.8 times the level of normal cells. The data showed that 3CsA-siRNA nanomicelle materials with different targets had a desirable effect of downregulating intracellular inflammatory genes.

### 8.3 Therapeutic Effects of 3CsA-siRNA Conjugate Nanomicelles Targeting Different Targets on Xerophthalmia

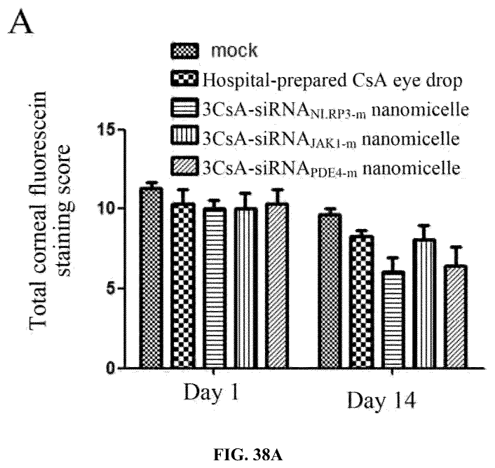

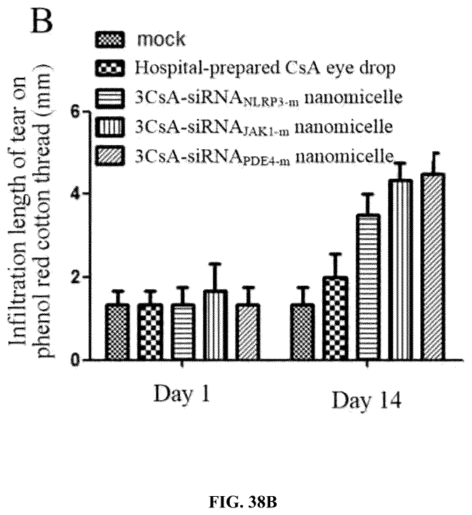

The ocular xerophthalmia model was established in 6-8 weeks old mice (C57BL/6, Suzhou Xishan Biotechnology Co., Ltd.) using 0.2% benzalkonium chloride. 5 μL of 0.2% benzalkonium chloride was dripped into each eye of the mouse, once in the morning and once in the evening every day for two weeks. After successful modeling, the hospital-prepared CsA eye drops (400 UM CsA, provided by the Eye, Ear, Nose and Throat Hospital Affiliated to Fudan University School of Medicine, Shanghai) and 3CsA-siRNA_{NLRP3-m} nanomicelles, 3CsA-siRNA_{JAK1}-m nanomicelles, and 3CsA-siRNA_{PDE4-m} nanomicelles (the CsA concentration in each group of materials was 400 μM) were used for eye drops treatment, 5 μL per eye, once in the morning and once in the evening. Sodium fluorescein staining and phenol red cotton thread tear secretion test were conducted on days 0 and 14 of treatment to evaluate the therapeutic effect. The experimental results were shown in FIGS. 38A-38B.

The results of sodium fluorescein staining showed that the eye score of mice in the untreated control group dropped from 12 points to about 10 points, and the infiltration length of tear on phenol red cotton thread was about 1 mm, with no obvious improvement; the eye score of mice treated with CsA eye drops dropped from 10 points to about 8 points, and the infiltration length of tear on phenol red cotton thread increased from 1 mm to about 2 mm; the sodium fluorescein staining score of mice in the siRNA nanomicelle material group with different targets dropped from about 10 points to about 7 points, and the infiltration length of tear on phenol red cotton thread increased from 1 mm to 3 mm to 4.5 mm. The data showed that drug-nucleic acid conjugate nanomicelles targeting different intracellular inflammatory targets showed significant therapeutic effects on xerophthalmia in mice over a two-week period.

### Example 9 the Treatment of Xerophthalmia with Triple CsA-ASO Conjugate Nanomicelles Targeting Different Targets of IL-17, IL-1β, IL-23, and TNF-α

9.1 Preparation of Conjugates of Three CsA Molecules and ASOs with Different Targets and their Self-Assembled Nanomicelles

In this example, the ASO targeting genes of different inflammatory factors were purchased from Shanghai Sangon Biotech Co., Ltd., and 3 DBCO modifications were introduced at the 3′ end of the ASO to enable efficient coupling with the azide group modified on CsA-N₃ through a click reaction, and the ASO sequences were as follows:

the ASO sequences targeting different human inflammatory factor genes were:

IL-17 human (3DBCO-ASO_{IL-17-h})
(SEQ ID NO: 51)
5′-TCGGACTAAACTCATTAGAGTT/iDBCOdT/T/iDBCOdT/
TT-DBCO-3′
IL-1β human (3DBCO-ASO_{IL-1β-h})
(SEQ ID NO: 52)
5′-GGTACTTCTGCCATGGCTGCTT/iDBCOdT/T/iDBCOdT/
TT-DBCO-3′
IL-23 human (3DBCO-ASO_{IL-23-h})
(SEQ ID NO: 53)
5′-CATTACAGCTCTGCTCCCCAGCATCTT/iDBCOdT/T/
iDBCOdT/TT-DBCO-3′
TNF-α human (ASO_{TNF-α-h})
(SEQ ID NO: 54)
5′-CAGTGCTCATGGTGTCTT/iDBCOdT/T/iDBCOdT/
TT-DBCO-3′

the ASO sequences targeting different mouse inflammatory factor genes were:

IL-17 mouse (3DBCO-ASO_{IL-17-m})
(SEQ ID NO: 55)
5′-TTCAGGACCAGGATCTCTTGCTT/iDBCOdT/T/
iDBCOdT/TT-DBCO-3′
IL-1β mouse (3DBCO-ASO_{IL-1β-m})
(SEQ ID NO: 56)
5′-ATGAGTGACACTGCCTTCCTT/iDBCOdT/T/
iDBCOdT/TT-DBCO-3′
IL-23 mouse (3DBCO-ASO_{IL-23-m})
(SEQ ID NO: 57)
5′-CCGTATCCAGTGTGGTGATGGTTGTTT/iDBCOdT/
T/iDBCOdT/TT-DBCO-3′
TNF-α mouse (3DBCO-ASO_{TNF-α-m})
(SEQ ID NO: 58)
5′-AACCCATCGGCTGGCACCACTT/iDBCOdT/T/
iDBCOdT/TT-DBCO-3′

The synthesis method of conjugates of the small molecule drug and the ASOs for different targets was as follows: 30 times the molar equivalent of CsA-N₃ was dissolved in DMSO, 5 OD of 3DBCO-ASOx with different targets were added to make the reaction concentration 200 μM, and then placed at 50° C. for shaking reaction for 24 h. After adding water, the excess CsA-N₃ in the reaction was removed by extraction with ethyl acetate, and the 3CsA-ASO_{IL-17}, 3CSA-ASO_{IL-18}, 3CSA-ASO_{LL-23}, and 3CsA-ASO_{TNF-α} conjugate molecules were obtained after concentration and evaporation. The conjugate molecules were redissolved with 50 μL DMSO solution and dropped into 500 μL PBS for assembly to form 3CsA-ASO_{IL-17} nanomicelles, 3CsA-ASO_{IL-18} nanomicelles, 3CsA-ASO_{IL-23} nanomicelles and 3CsA-ASO_{TNF-α} nanomicelles, and then dialyzed in PBS to remove DMSO to obtain different nanomicelle samples dispersed in PBS solution.

### 9.2 Knockdown Effect of Nanomicelles for Different Targets on Corresponding Genes

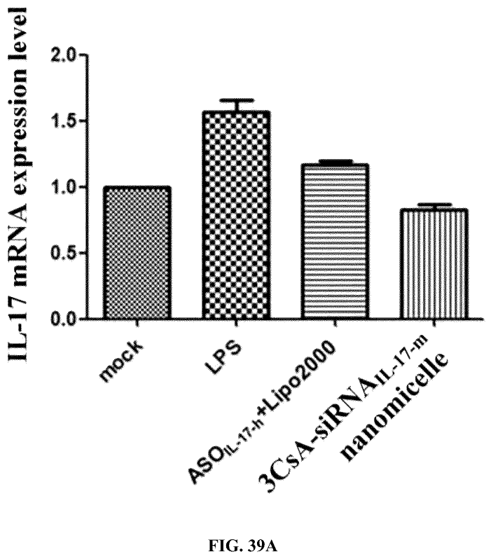

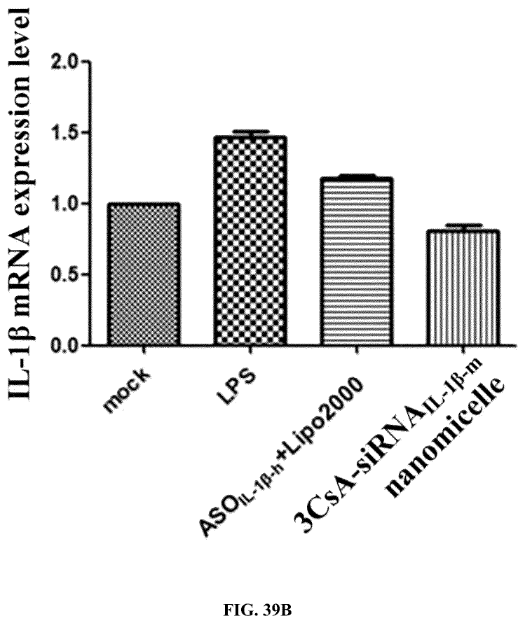

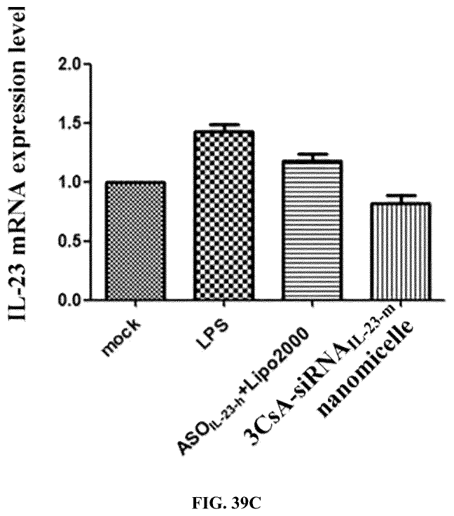

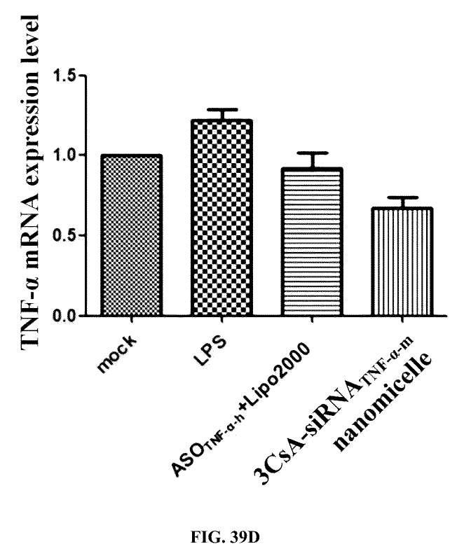

HCECs were inoculated in a 6-well plate at a density of 1×10⁵ cells/well and cultured overnight until adherence. The cells were then stimulated with 100 ng/mL LPS for 12 h. After removing the supernatant, 1 mL of Opti-MEM medium containing 3CsA-ASO_{IL-17-h} nanomicelles, 3CsA-ASO_{IL-18-h} nanomicelles, 3CsA-ASO_{IL-23-h} nanomicelles, and 3CsA-ASO_{TNF-α-h} nanomicelles (all containing 10 UM ASO) were added, respectively, while control group was 1 mL of Opti-MEM medium containing different target ASO and Lipo2000 complex (the ASO concentration was 10 μM). After incubation at 37° C. for 6 h, the cells were transferred into DMEM medium and incubated at 37° C. for another 48 h. The cells were collected and RNA was extracted for RT-qPCR to evaluate the expression of mRNA of different targets in HCECs. The experimental results were shown in FIGS. 39A-39D.

After LPS stimulation, the mRNA expression levels of different inflammatory factors were upregulated, which was about 1.3 to 1.5 times that of normal cells. After adding Lipo2000 to transfect ASOs of different targets, the expression of different inflammatory factors was reduced to 0.8-1.1 times the normal cell level; after adding nanomicelle materials of different targets, the expression of different inflammatory factors was reduced to 0.6-0.8 times the normal cell level. The data showed that 3CsA-ASO nanomicelle materials with different targets had a desirable effect of downregulating inflammatory factor genes.

### 9.3 Inhibitory Effects of Different Target Nanomicelles on Inflammatory Factors

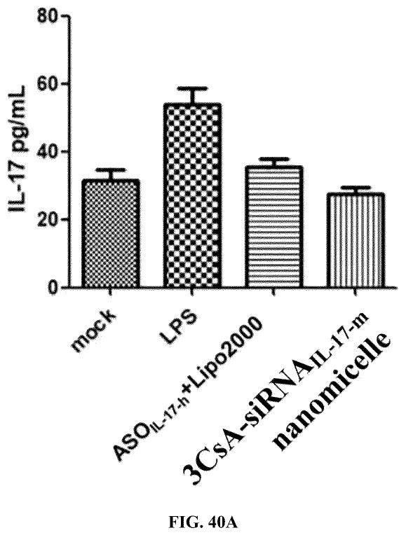

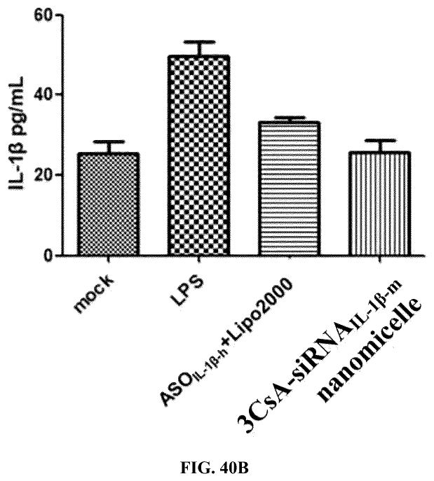

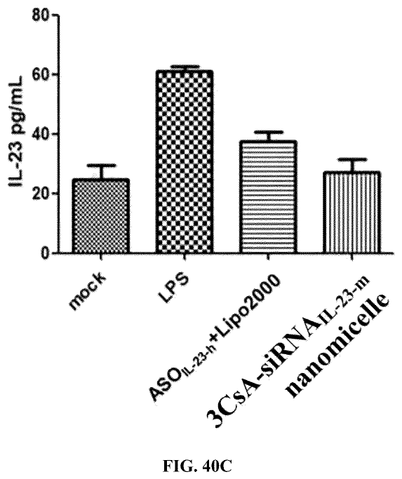

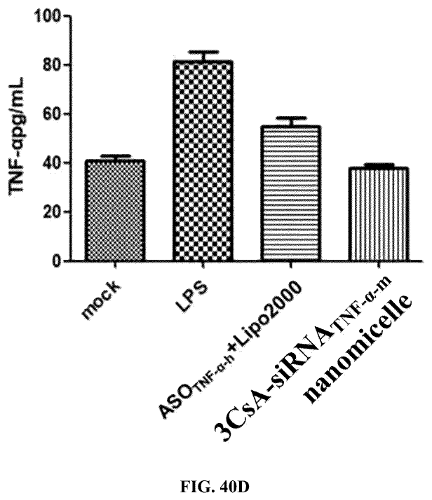

HCECs were inoculated in a 12-well plate at a density of 5×10⁴ cells/well and cultured overnight until adherence. The cells were then stimulated with 100 ng/ml LPS for 12 h. After removing the supernatant, 1 mL of Opti-MEM medium containing 3CsA-ASO_{IL-17-h} nanomicelles, 3CsA-ASO_{IL-18-h} nanomicelles, 3CsA-ASO_{IL-23-h} nanomicelles, and 3CsA-ASO_{TNF-α-h} nanomicelles (all containing 10 UM ASO) were added, respectively, while control group was 1 mL of Opti-MEM medium containing different target ASO and Lipo2000 complex (the ASO concentration was 10 μM). After incubation at 37° C. for 6 h, the cells were transferred into DMEM medium and incubated at 37° C. for another 48 h. The cells were collected and RNA was extracted for ELISA to detect the expression of different inflammatory factors in HCECs, and experimental results were shown in FIGS. 40A-40D.

After LPS stimulation, the expression of various inflammatory factors in HCECs increased significantly, with the increase range being about 1.5 to 2.5 times that of normal cells; after adding different target ASOs transfected with Lipo2000, the expression of inflammatory factors decreased; the expression of inflammatory factors in HCECs treated with various nanomicelle materials decreased the most and returned to the level of normal cells. The data showed that 3CsA-ASO nanomicelle materials with different targets had a desirable effect of downregulating inflammatory factors.

### 9.4 Therapeutic Effects of Nanomicelles Targeting Different Targets on Xerophthalmia

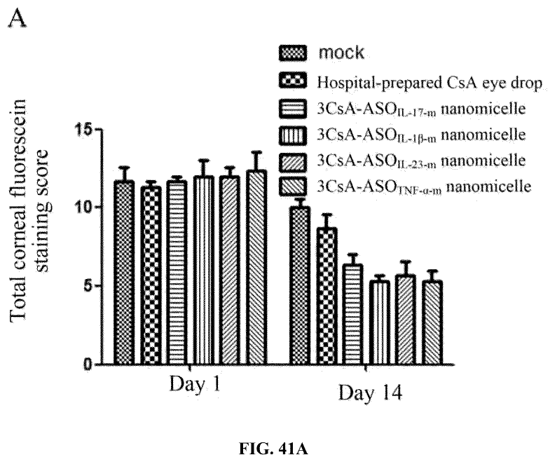

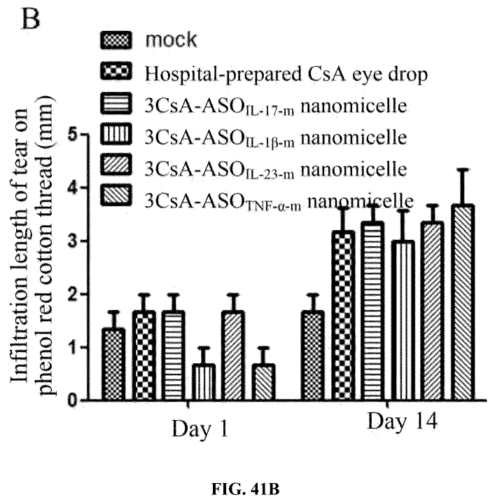

The ocular xerophthalmia model was established in 6-8 weeks old mice (C57BL/6, Suzhou Xishan Biotechnology Co., Ltd.) with 0.2% benzalkonium chloride. 5 μL of 0.2% benzalkonium chloride was dripped into each eye of the mice, once in the morning and once in the evening every day, for two consecutive weeks. After successful modeling, the hospital-prepared CsA eye drops (400 UM CsA, provided by the Eye, Ear, Nose and Throat Hospital Affiliated to Fudan University School of Medicine, Shanghai) and 3CsA-ASO_{IL-17-m} nanomicelles, 3CsA-ASO_{IL-18-m} nanomicelles, 3CsA-ASO_{IL-23-m} nanomicelles, and 3CsA-ASO_{TNF-α-m} nanomicelles (the CsA concentration in each group of materials was 400 μM) were used for eye drops treatment, 5 μL per eye, once in the morning and once in the evening. Sodium fluorescein staining and phenol red cotton thread tear secretion test were conducted on days 0 and 14 of treatment to evaluate the therapeutic effect. The experimental results were shown in FIGS. 41A-41B.

The results of sodium fluorescein staining showed that the eye score of mice in the untreated control group dropped from 11 points to about 10 points, and the infiltration length of tear on phenol red cotton thread increased from 1.5 mm to about 1.8 mm, with no significant improvement; the eye score of mice treated with CsA eye drops dropped from 11 points to about 8 points, and the infiltration length of tear on phenol red cotton thread increased from 1.5 mm to about 3 mm; the sodium fluorescein staining score of mice in the 3CsA-ASO nanomicelle material group with different targets dropped from about 11 points to about 5.5 points, and the infiltration length of tear on phenol red cotton thread increased from 1 mm to about 3 mm. The data showed that drug-ASO conjugate nanomicelles targeting different inflammatory factor targets showed significant therapeutic effects on xerophthalmia in mice within two weeks.

### Example 10 the Treatment of Xerophthalmia with CsA-Grafted ASO Self-Assembled Nanomicelles Targeting Different Targets of NLRP3, JAK1, and PDE4

### 10.1 Synthesis and Characterization of Bromo-CsA (CsA-Br)

The synthesis method of CsA-Br was conducted according to the corresponding steps in Example 6.

10.2 Grafting of CsA-Br and PS-ASOs with Different Targets and Preparation and Characterization of their Nanomicelles

In this example, the ASO sequences used to target different inflammatory pathway genes in cells were purchased from Shanghai Sangon Biotech Co., Ltd. The phosphate backbone of the ASO was modified with thio (PS) to enable ASO to undergo substitution with a bromoacetyl bromide group modified on the CsA-Br for efficient coupling. The sequence was as follows:

the ASO sequences targeting different human inflammatory factor genes were:

NLRP3 human (20PS-ASO_{NLRP3-h})
(SEQ ID NO: 59)
5′-AGCTGCTGCCCCGACCCAAACCTTTTTT*T*T*T*T*T*
T*T*T*T*T*T*T*T*T*T*T*T*T*T*T-3′
JAK1 human (20PS-ASO_{JAK1-h})
(SEQ ID NO: 60)
5′-TTCGTCATCCTTGTAATCCATTTTTTT*T*T*T*T*T*T*
T*T*T*T*T*T*T*T*T*T*T*T*T*T-3′
PDE4 human (20PS ASO_{PDE4-h})
(SEQ ID NO: 61)
5′-TTGATTTCCAGACCGACTTTTTT*T*T*T*T*T*T*T*T*
T*T*T*T*T*T*T*T*T*T*T*T-3′

the ASO sequences targeting different mouse inflammatory factor genes were:

NLRP3 mouse (20PS-ASO_{NLRP3-m})
(SEQ ID NO: 62)
5′-GATTTAAGTACATTTCACCTTTTTT*T*T*T*T*T*
T*T*T*T*T*T*T*T*T*T*T*T*T*T*T-3′
JAK1 mouse (20PS-ASO_{JAK1-m})
(SEQ ID NO: 63)
5′-TTGGAGAATGTCGCCATACAGTTTTTT*T*T*T*T*
T*T*T*T*T*T*T*T*T*T*T*T*T*T*T*T-3′
PDE4 mouse (20PS ASO_{PDE4-m})
(SEQ ID NO: 64)
5′-TTGTCCTCTCTACACGCTCATTTTTT*T*T*T*T*T*T*
T*T*T*T*T*T*T*T*T*T*T*T*T*T-3′

The synthesis method was as follows: 400 times the molar equivalent of CsA-Br was dissolved in DMSO, 5 OD of 20PS-ASO with different targets were added to make the reaction concentration 200 μM, and then placed at 50° C. for shaking reaction for 24 h. After adding water, the excess CsA-Br in the reaction was removed by extraction with ethyl acetate, and the 20CsA-ASO_{NLRP3}, 20CSA-ASO_{JAK1}, and 20CsA-ASO_{PDE4} conjugate molecules were obtained after concentration and evaporation. The conjugate molecules were redissolved with 50 μL DMSO solution and dropped into 500 μL PBS for assembly to form 20CsA-ASO_{NLRP3} nanomicelles, 20CsA-ASO_{JAK1} nanomicelles and 20CsA-ASO_{PDE4} nanomicelles, and then dialyzed in PBS to remove DMSO to obtain different nanomicelle samples dispersed in PBS solution.

### 10.3 Regulatory Effects of Nanomicelles for Different Targets on Target Gene

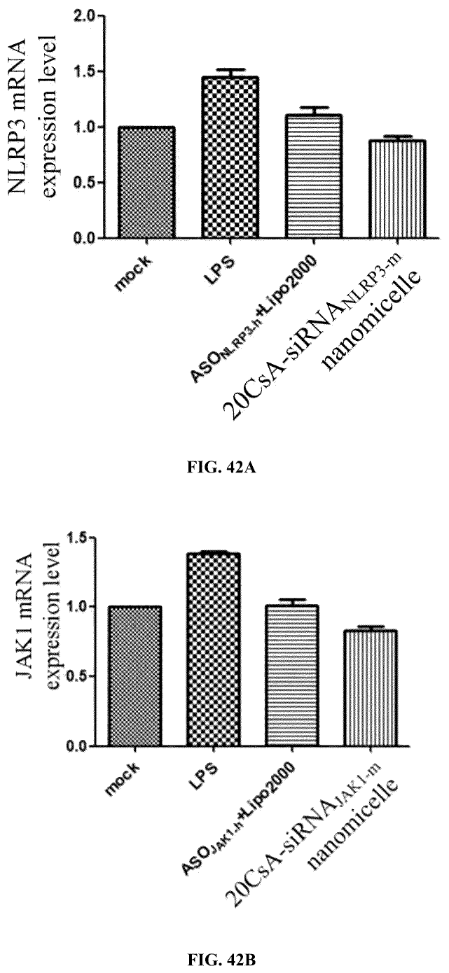

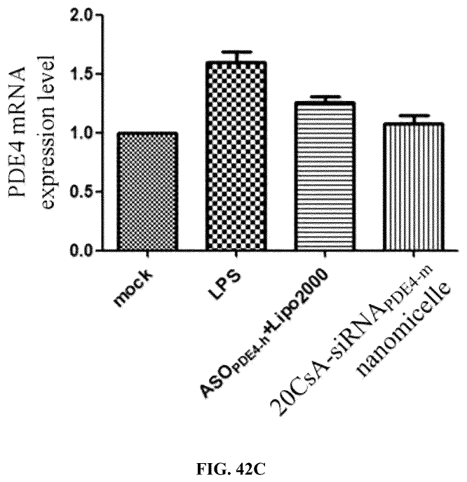

HCECs were inoculated in a 6-well plate at a density of 1×10⁵ cells/well and cultured overnight until adherence. The cells were then stimulated with 100 ng/ml LPS for 12 h. After removing the supernatant, 1 mL of Opti-MEM medium containing 20CsA-ASO_{NLRP3-h} nanomicelles, 20CsA-ASO_{JAK1-h} nanomicelles, and 20CsA-ASO_{PDE4-h} nanomicelles (all containing 10 UM ASO) were added, respectively, while control group was 1 mL of Opti-MEM medium containing different target ASO and Lipo2000 complex (the ASO concentration was 10 μM). After incubation at 37° C. for 6 h, the cells were transferred into DMEM medium and incubated for another 48 h. The cells were collected and RNA was extracted for RT-qPCR to evaluate the expression of mRNA of different targets in HCECs. The experimental results were shown in FIGS. 42A-42C.

After LPS stimulation, the mRNA expression levels of different inflammatory factors were upregulated, which was about 1.3 to 1.5 times that of normal cells. After adding Lipo2000 to transfect ASOs of different targets, the expression of different inflammatory factors was reduced to 1-1.2 times the normal cell level; after adding nanomicelle materials of different targets, the expression of different inflammatory factors was reduced to about 0.8 times the normal cell level. The data showed that 20CsA-ASO nanomicelle materials with different targets had a desirable effect of downregulating inflammatory factor genes.

### 10.4 Therapeutic Effects of Nanomicelles Targeting Different Targets on Xerophthalmia

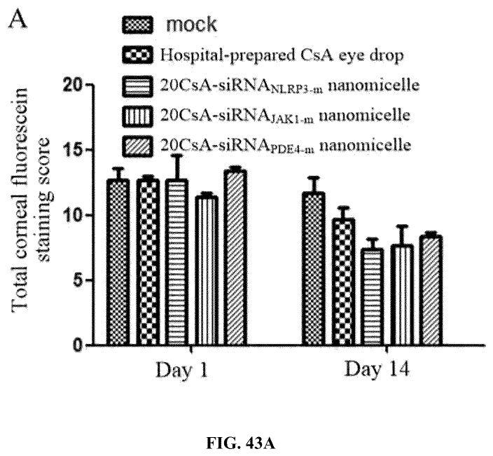

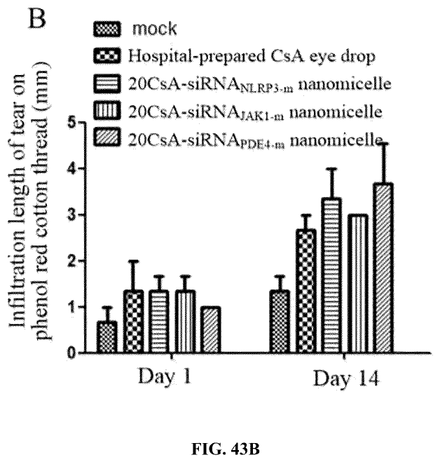

The ocular xerophthalmia model was established in 6-8 weeks old mice (C57BL/6, Suzhou Xishan Biotechnology Co., Ltd.) using 0.2% benzalkonium chloride. 5 μL of 0.2% benzalkonium chloride was dripped into each eye of the mouse, once in the morning and once in the evening every day for two weeks. After successful modeling, the hospital-prepared CsA eye drops (400 UM CsA, provided by the Eye, Ear, Nose and Throat Hospital Affiliated to Fudan University School of Medicine, Shanghai) and 20CsA-ASO_{NLRP3-m} nanomicelles, 20CsA-ASO_{JAK1}-m nanomicelles, and 20CsA-ASO_{PDE4-m} nanomicelles (the CsA concentration in each group of materials was 400 μM) were used for eye drops treatment, 5 L per eye, once in the morning and once in the evening. Sodium fluorescein staining and phenol red cotton thread tear secretion test were conducted on days 0 and 14 of treatment to evaluate the therapeutic effect. The experimental results were shown in FIGS. 43A-43B.

The results of sodium fluorescein staining showed that the eye score of mice in the untreated control group dropped from 13 points to about 12 points, and the infiltration length of tear on phenol red cotton thread increased from 0.5 mm to about 1 mm, with no significant improvement; the eye score of mice treated with CsA eye drops dropped from 13 points to about 10 points, and the infiltration length of tear on phenol red cotton thread increased from 1.5 mm to about 2.5 mm; the sodium fluorescein staining score of mice in the 20CsA-ASO nanomicelle material group with different targets dropped from about 13 points to about 7.5 points, and the infiltration length of tear on phenol red cotton thread increased from 1 mm to about 3.5 mm. The data showed that drug-ASO conjugate nanomicelles targeting different intracellular inflammatory targets showed significant therapeutic effects on xerophthalmia in mice within two weeks.

### Example 11 the Treatment of Xerophthalmia with Rapamycin-ASO Conjugate and its Nanomicelle Assembly

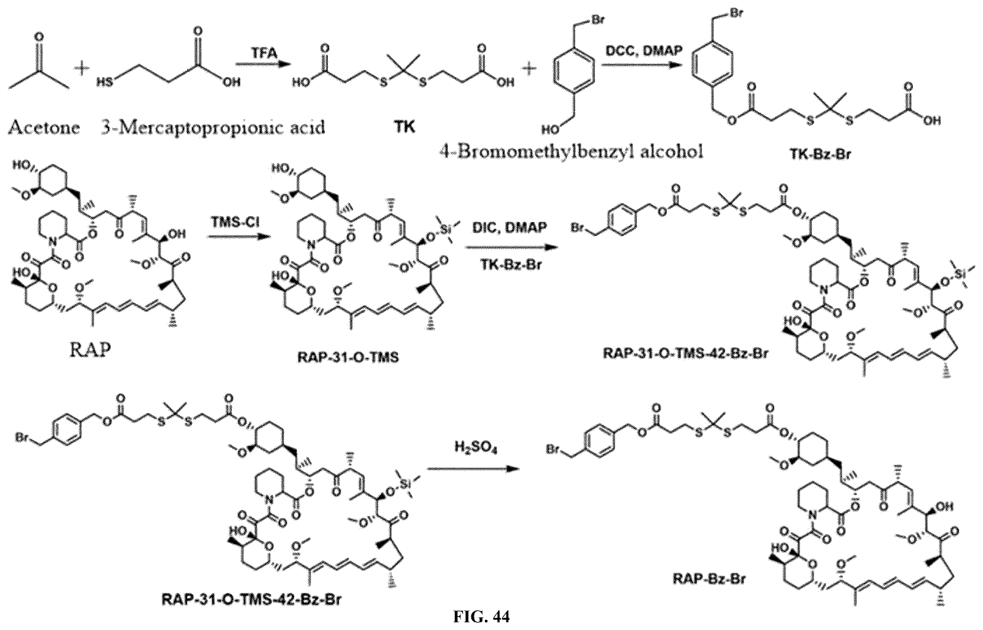

### 11.1 Synthesis of Benzyl Bromide-Modified Rapamycin (RAP-Bz-Br), as Shown in FIG. 44

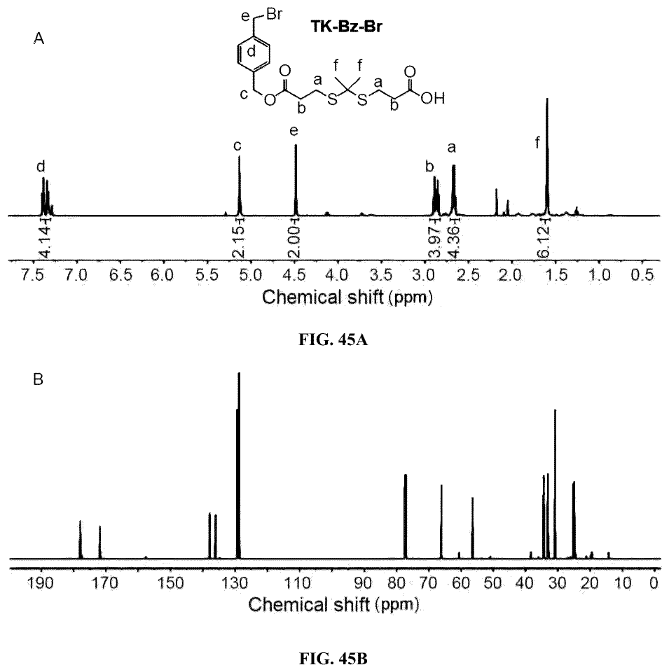

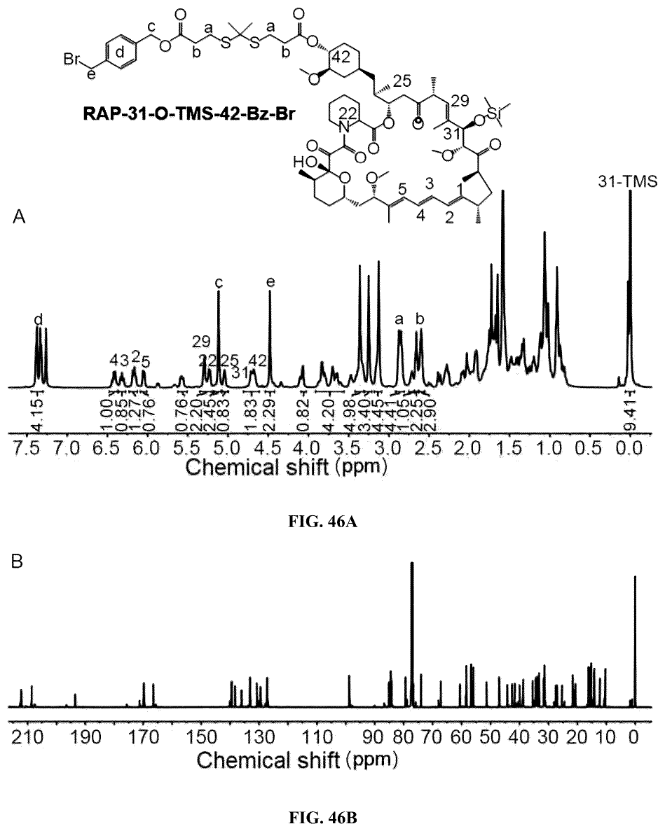

a) Synthesis of benzyl bromide-modified thioketal (TK-Bz-Br). 3-Mercaptopropionic acid (2.2 eq.), acetone (1 eq.), and a catalytic amount of TFA were stirred at room temperature overnight, and then a resulting mixture was cooled in an ice bath for crystallization. The prepared crystals were filtered and further washed with n-hexane, and then dried in a vacuum oven at 45° C. overnight to obtain a white product, thereby obtaining a thioketal compound (compound TK) having reactive oxygen species (ROS) responsive cleavage characteristics. 4-Bromomethylbenzyl alcohol (1 eq.) and the compound TK (3 eq.) were dissolved in ultra-dry DCM, and then 4-dimethylaminopyridine (DMAP, 0.5 eq.) was added. After stirring at room temperature for 5 min, dicyclohexylcarbodiimide (DCC, 1.2 eq., dissolved in ultra-dry DCM) was added dropwise. During the reaction, silica gel thin layer chromatography was conducted to monitor a reaction progress. After the reaction, impurities were removed by filtration, the solvent in the filtrate was evaporated to dryness using a rotary evaporator, and the product was purified by silica gel column chromatography using a mixed solution of petroleum ether/ethyl acetate as an eluent to obtain the compound TK-Bz-Br with a final yield of 65%. The product was analyzed and detected by NMR spectroscopy, and the NMR spectrum and the attribution of each peak were shown in FIGS. 45A-45B.
b) 1 g of rapamycin (RAP, 1 eq.) was dissolved in 20 mL of ethyl acetate. The mixture was placed in an ice bath and stirred, and imidazole (10 eq.) was added and stirred until dissolved. Trimethylsilyl chloride (TMS-Cl) was dissolved in 5 mL of ethyl acetate and then dripped into the above reaction solution dropwise. The mixture was stirred for about 2 h to obtain an intermediate product (compound RAP-31, 42-bis-O-TMS) in which positions 31 and 42 of rapamycin were both protected by TMS. 6 mL of 0.5 M sulfuric acid (H₂SO₄) solution was added dropwise into the above mixture and placed in an ice bath overnight. 60 mL of ethyl acetate was added to the reaction solution, and extraction was conducted with saturated sodium bicarbonate (NaHCO₃, 2 times) and deionized water (3 times), and a pH value of the aqueous solution layer was controlled at 6.0 to 7.0. The ethyl acetate solution was collected by extraction, dried over anhydrous sodium sulfate, and then evaporated to dryness by rotary evaporation to obtain rapamycin in which only the 31-position hydroxyl group was protected by TMS (compound RAP-31-O-TMS). The compound RAP-31-O-TMS had a yield of 90%, and the m/z obtained by mass spectrometry was 1,008.61, which could be attributed to [M+Na]⁺. The compound TK-Bz-Br (1 eq.) obtained in a) and the compound RAP-31-O-TMS (0.8 eq.) were dissolved in anhydrous DCM, and DMAP (0.5 equivalent) was added at room temperature and stirred until dissolved. The mixture was added with diisopropylcarbodiimide (DIC, 2 eq.) dissolved in DCM and stirred at room temperature overnight. The product was purified by silica gel column chromatography to obtain a compound RAP-31-O-TMS-42-Bz-Br. The compound RAP-31-O-TMS-42-Bz-Br had a yield of 35%, and the m/z obtained by mass spectrometry was 1,419.64/1,421.64, which could be attributed to [M+NH₄]⁺. The results of H NMR and C NMR were shown in FIGS. 46A-46B. 0.4 g of the compound RAP-31-O-TMS-42-Bz-Br was dissolved in a mixed solution of 10 mL of acetonitrile and 20 mL of ethyl acetate, 3 mL of 1 M H₂SO₄ was slowly added dropwise and stirred continuously for 2 h, and finally 50 mL of ethyl acetate was added, followed by washing twice with 50 ml of water and extraction. The ethyl acetate layer was collected, dried over anhydrous sodium sulfate, and then rotary evaporated to dryness to obtain the final product compound RAP-Bz-Br, where the compound RAP-Bz-Br had a yield of 85%, and the m/z obtained by mass spectrometry was 1,347.59/1,349.59, which could be attributed to [M+NH₄]⁺.

### 11.2 Synthesis of 10 Rapamycin Molecules-NFKBIZ ASO Conjugate (10RAP-ASO_{NFKBIZ})

In this example, the ASO sequences used to target NFKBIZ gene were purchased from Shanghai Sangon Biotech Co., Ltd. The phosphate backbone of the ASO was modified with thio (PS) to enable undergo substitution with a bromoacetyl bromide group modified on the CsA-Br for efficient coupling. The sequence was as follows:

the sequence of the ASO targeting human NFKBIZ gene (ASO_{NFKBIZ-h}) was

Human:
(SEQ ID NO: 65)
5′-ATCAGACAACGAATCGGGCTTTT*T*T*T*T*T*T*T*
T*T*T-3′;

the sequence of the ASO targeting mouse NFKBIZ gene (ASO_{NFKBIZ-m}) was:

Mouse:
(SEQ ID NO: 66)
5′-AATACTGGTACATTGACGCCTTTT*T*T*T*T*T*T*
T*T*T*T-3′.

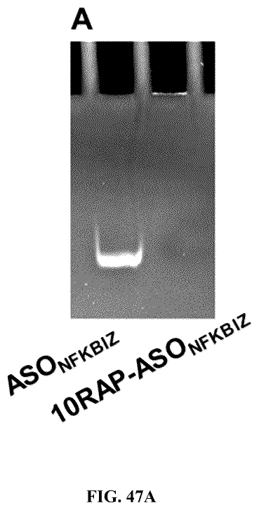

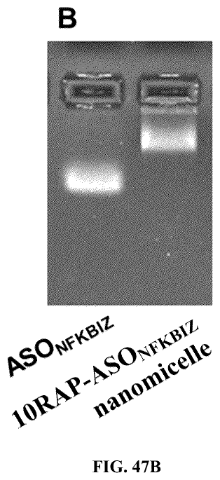

The synthesis method of 10 rapamycin molecules-NFKBIZ ASO conjugate (10RAP-ASO_{NFKBIZ}) was as follows: 2.2 mg of the compound RAP-Bz-Br (1.63 μmol) was dissolved in 100 μL of DMSO, and then 5 OD of ASO (20.55 nmol) was added, and the mixture was reacted by shaking at 50° C. for 1 h. Water was added, and the excess RAP-Bz-Br in the reaction was removed by extraction with ethyl acetate, and then the aqueous solution was concentrated and evaporated to dryness to obtain the 10RAP-ASO_{NFKBIZ} conjugate molecule. The obtained product was verified by 15% denatured polyacrylamide gel electrophoresis to confirm that the ASO was successfully grafted with rapamycin, as shown in FIGS. 47A-47B.

### 11.3 Preparation and Characterization of 10 Rapamycin -NFKBIZ ASO Conjugate (10RAP-ASO_{NFKBIZ}) Nanomicelles

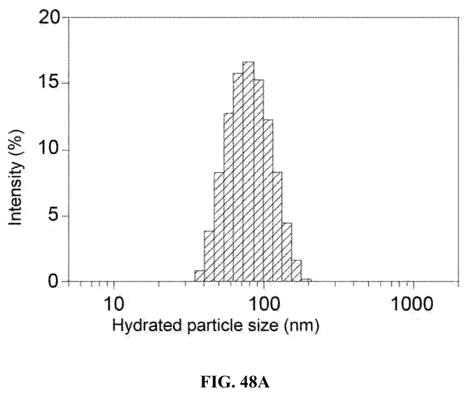

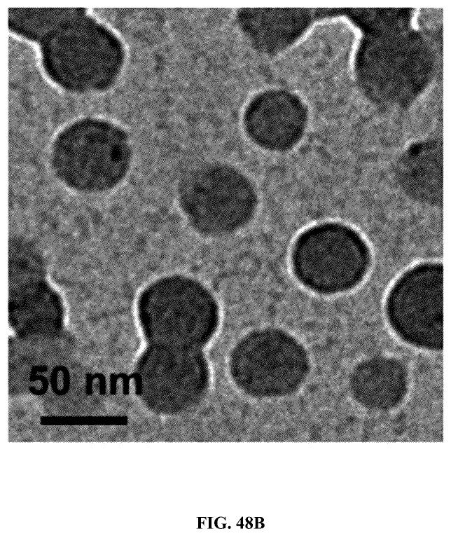

10RAP-ASO_{NFKBIZ} conjugate (10 OD ASO) was dissolved in 100 μL of DMSO and added dropwise to 300 μL of PBS with continuous stirring. The solution was dialyzed overnight in a dialysis bag with PBS to remove DMSO. 10RAP-ASO_{NFKBIZ} nanomicelles were obtained by centrifugation at 3,000 rpm for 5 min. The hydrated particle size and morphology of the nanomicelles were characterized by dynamic light scattering (DLS) and transmission electron microscopy (TEM), as shown in FIGS. 48A-48B.

### 11.4 Gene Regulation of 10RAP-ASO_{NFKBIZ-h} Nanomicelles

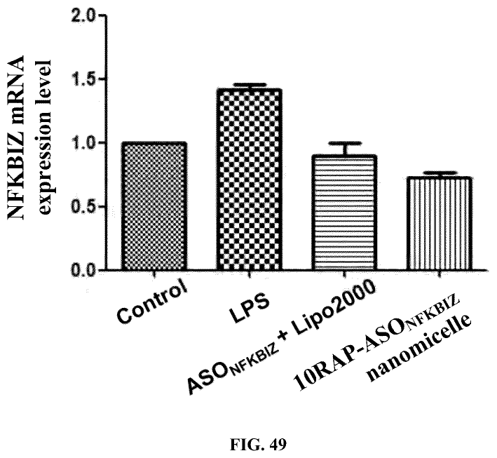

HCECs were inoculated in a 6-well plate at a density of 1×10⁵ cells/well and cultured until adherence. The cells were then stimulated with 100 ng/mL LPS overnight. 1 mL of Opti-MEM medium ASO_{NFKBIZ-h}+Lipo2000 complex and 10RAP-ASO_{NFKBIZ-h} (all containing 10 UM ASO) were added, respectively. After incubation at 37° C. for 6 h, the cells were transferred into DMEM medium and incubated at 37° C. for another 48 h. The cells were collected and RNA was extracted for RT-qPCR to analyze the expression level of the NFKBIZ mRNA in HCECs, and experimental results were shown in FIG. 49. The expression level of NFKBIZ mRNA in HCECs after LPS stimulation was about 1.4 times that of normal cells. After ASO_{NFKBIZ-h} was transfected with Lipo2000, its expression level was reduced to 0.75 times that of normal cells. After adding 10RAP-ASO_{NFKBIZ-h} nanomicelles for co-incubation, the expression of NFKBIZ mRNA was reduced to about 0.6 times that of normal cells. 10RAP-ASO_{NFKBIZ-h} nanomicelles could effectively regulate the expression of NFKBIZ gene in HCECs and significantly knock down the expression of NFKBIZ mRNA.

### 11.5 Therapeutic Effect 10RAP-ASO_{NFKBIZ-m} Nanomicelles on Xerophthalmia

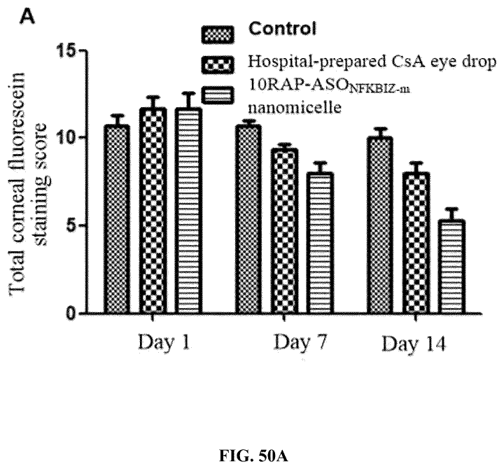

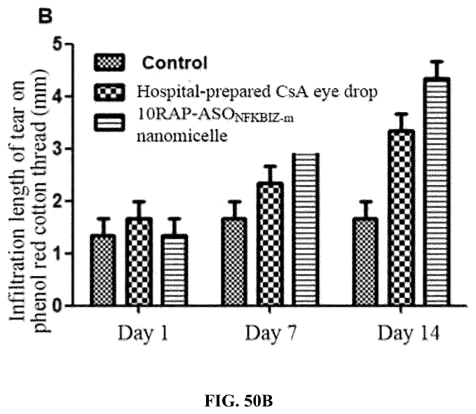

The ocular xerophthalmia model was established in 6-8 weeks old mice (C57BL/6, Suzhou Xishan Biotechnology Co., Ltd.) with 0.2% benzalkonium chloride. 5 μL of the 0.2% benzalkonium chloride was dripped into each eye of the mice, once in the morning and once in the evening every day, for two consecutive weeks. After successful modeling, different drugs were used for eye drop treatment, and the groups were divided into: an untreated group (mock), hospital-prepared CsA eye drops (400 UM CsA, provided by the Eye, Ear, Nose and Throat Hospital Affiliated to Fudan University School of Medicine), and 10RAP-ASO_{NFKBIZ-m} nanomicelles (400 μM rapamycin). 5 μL was dropped into each eye once in the morning and once in the evening. Sodium fluorescein staining and phenol red cotton thread tear secretion test were conducted on days 0, 7, and 14 of treatment to evaluate the therapeutic effect. The experimental results were shown in FIGS. 50A-50B. For the untreated control group (control group), the sodium fluorescein staining score and the infiltration length of tear on phenol red cotton thread after 14 d of treatment were not much different from those before treatment, indicating that the eyes of untreated mice with xerophthalmia did not return to normal on their own. For the CsA eye drops group, the fluorescein sodium staining score was slightly reduced after treatment, and the tear secretion measured by phenol red cotton thread was increased, and the effect was more obvious with the increase of treatment time. However, after 14 days of treatment, the tear secretion measured by phenol red cotton thread was an average of 3 mm, which did not return to normal. The therapeutic effect was most significant in the 10RAP-ASO_{NFKBIZ-m} nanomicelle experimental group. After 14 d of treatment, the sodium fluorescein staining score was the lowest, with an average of 5, and the tear secretion was the highest, with an average of 4 mm.

### Example 12 the Treatment of Psoriasis with CsA Trimer (CsA₃)-NFKBIZ ASO covalent conjugate (CsA₃-ASO_{NFKBIZ}) and its Nanomicelle Assembly Structure

12.1 Synthesis of CsA₃-ASO_{NFKBIZ} Covalent Conjugate

The CsA₃-ASO_{NFKBIZ} covalent conjugate was the same as the conjugate in Example 5, and its specific synthesis steps referred to 5.1 to 5.2 in Example 5.

12.2 Preparation and Characterization of CsA₃-ASO_{NFKBIZ} Nanomicelles

The preparation and characterization steps of CsA₃-ASO_{NFKBIZ} nanomicelles referred to 5.3 in Example 5.

12.3 Regulatory Effect of CsA₃-ASO_{NFKBIZ-h} Nanomicelles on Target Gene

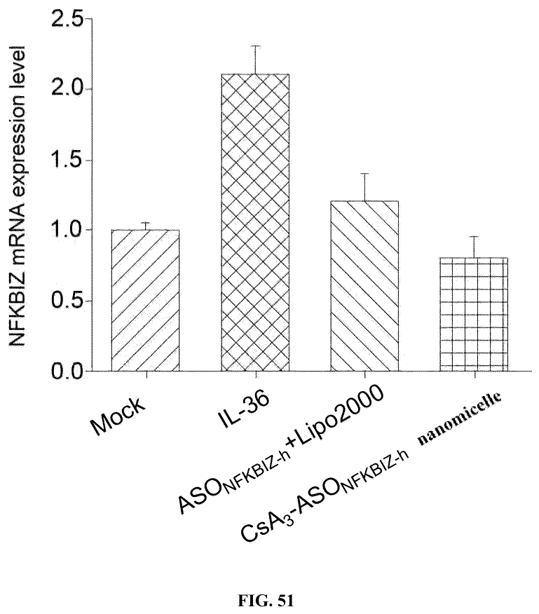

HaCaT cells from human keratinocyte cell line were inoculated in a 6-well plate at a density of 1×10⁵ cells/well and cultured until adherence. The cells were then stimulated with 100 ng/ml interleukin-36 (IL-36) for 2 h. After washing with 1×PBS buffer, 1 mL of Opti-MEM medium containing ASO_{NFKBIZ-h}+Lipo2000 complex and CsA₃-ASO_{NFKBIZ-h} nanomicelles (all containing 10 μM ASO_{NFKBIZ-h}) were added, respectively. After incubation with HaCaT cells for 6 h, the cells were transferred into fresh DMEM medium, while cells without any treatment or stimulated with IL-36 alone were used as a control, all cells were incubated at 37° C. for 48 h. The cells were collected and RNA was extracted for RT-qPCR to analyze the expression level of the NFKBIZ mRNA in HaCaT cells, and experimental results were shown in FIG. 51.

After IL-36 stimulation, the NFKBIZ mRNA expression of HaCaT cells increased to about 2.1 times that of normal cells. ASO_{NFKBIZ-h} after Lipo2000 transfection could downregulate it to about 1.2 times. CsA₃-ASO_{NFKBIZ-h} nanomicelles downregulated the NFKBIZ gene to about 0.8 times. Therefore, compared to single ASO_{NFKBIZ-h}, CsA₃-ASO_{NFKBIZ-h} nanomicelles had a better ability to down-regulate the expression of NFKBIZ gene.

12.4 Regulatory Effect of CsA₃-ASO_{NFKBIZ} Nanomicelles on Inflammatory Factors

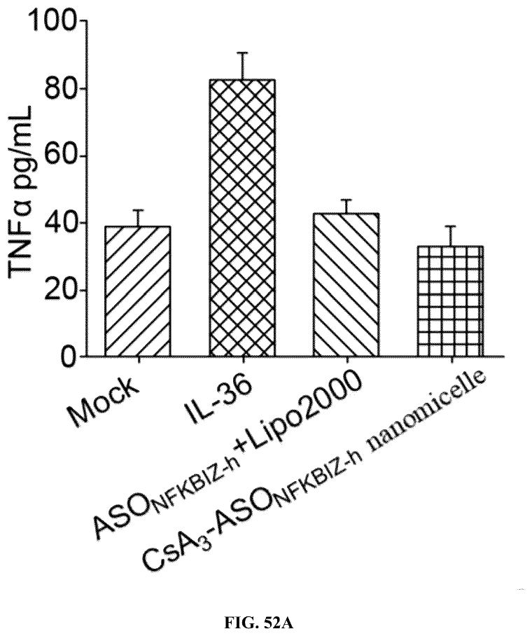

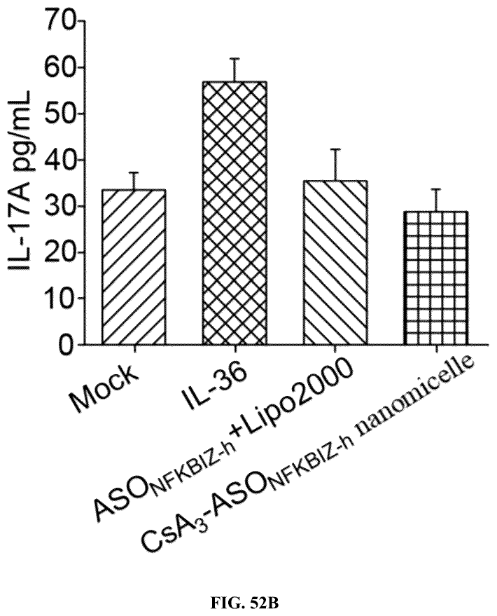

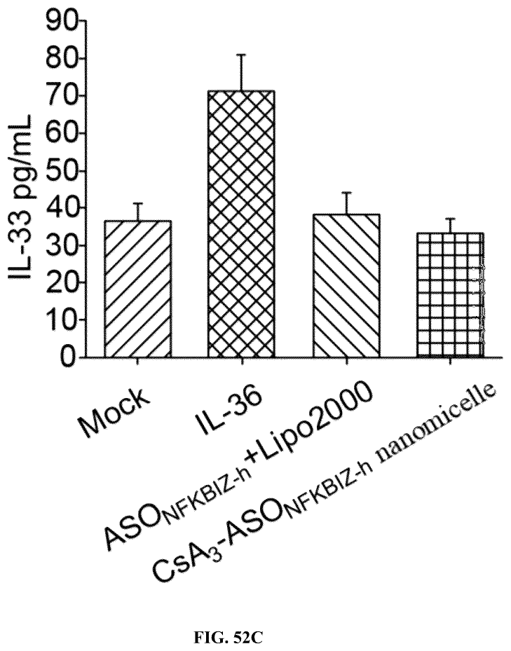

HaCaT cells from human keratinocyte cell line were inoculated in a 12-well plate at a density of 5×10⁴ cells/well and cultured until adherence. The cells were then stimulated with 100 ng/ml interleukin-36 (IL-36) for 2 h. After washing with 1×PBS buffer, 1 mL of Opti-MEM medium containing ASO_{NFKBIZ-h}+Lipo2000 complex and CsA₃-ASO_{NFKBIZ-h} nanomicelles (all containing 10 μM ASO_{NFKBIZ-h}) were added, respectively. After incubation with HaCaT cells for 6 h, the cells were transferred into fresh DMEM medium, while cells without any treatment or stimulated with IL-36 alone were used as a control, all cells were incubated at 37° C. for 48 h. The supernatant was collected and the secretion levels of TNFα, IL-17A, and IL-33 were detected using ELISA kit, and experimental results were shown in FIGS. 52A-52C.

After IL-36 stimulation, the levels of TNFα, IL-17A, and IL-33 in HaCaT cells increased to about 2.1 times, 1.7 times, and 2.0 times of those in unstimulated cells, respectively. ASO_{NFKBIZ-h} transfected with Lipo2000 could downregulate the levels of TNFα, IL-17A, and IL-33 to about 1.1 times, 1.0 times, and 0.9 times, respectively. CsA₃-ASO_{NFKBIZ-h} nanomicelles could downregulate the levels of TNFα, IL-17A, and IL-33 to about 0.8 to 0.9 times. Therefore, compared to a single ASO_{NFKBIZ-h}, CsA₃-ASO_{NFKBIZ-h} nanomicelles had a stronger inhibitory effect on inflammatory factors such as TNFα, IL-17A, and IL-33.

12.5 Therapeutic Effect of CsA₃-ASO_{NFKBIZ-m} Nanomicelles on Psoriasis

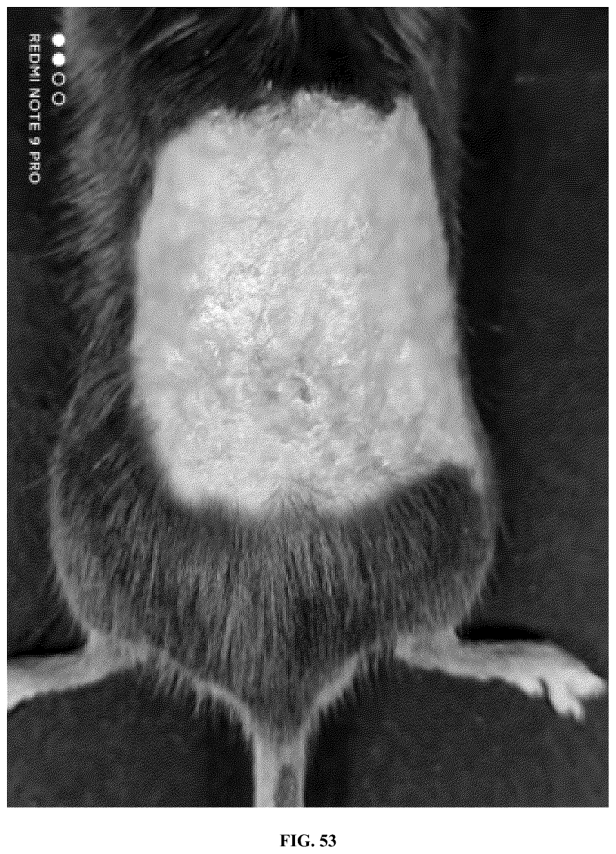

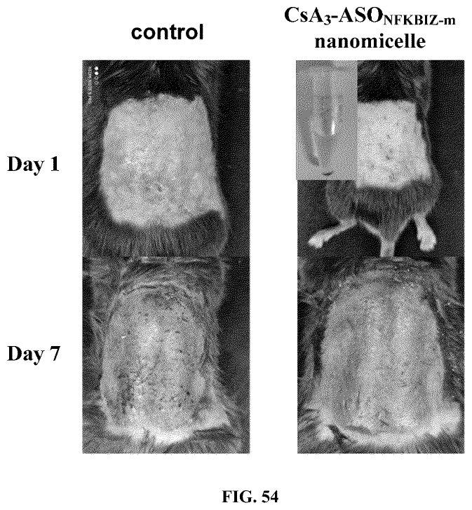

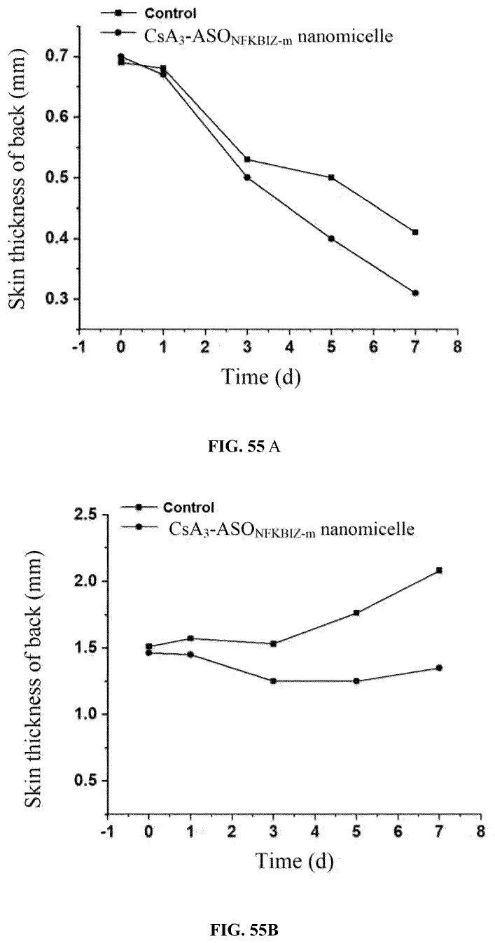

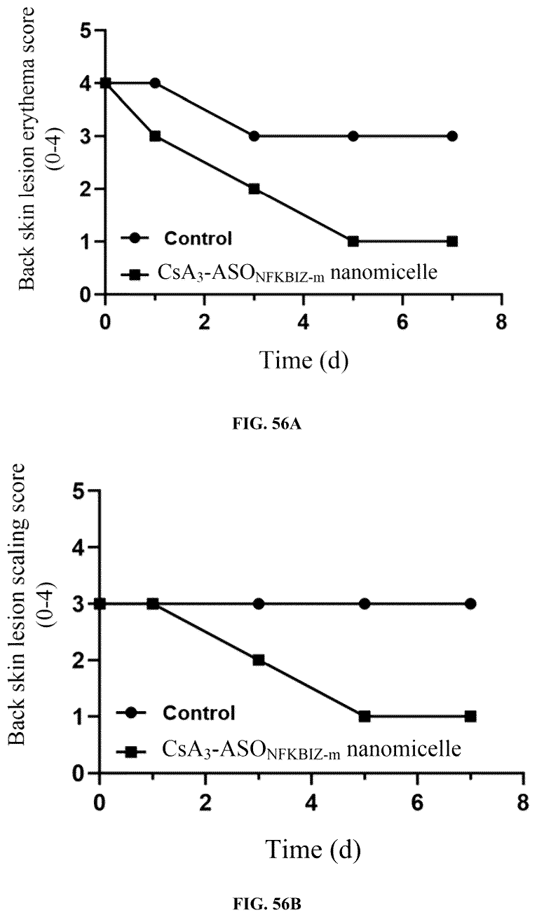

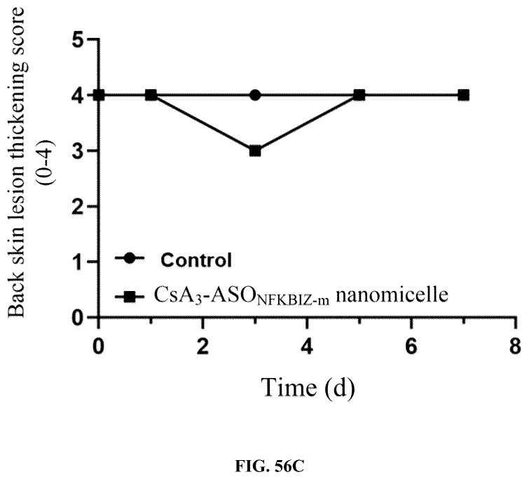

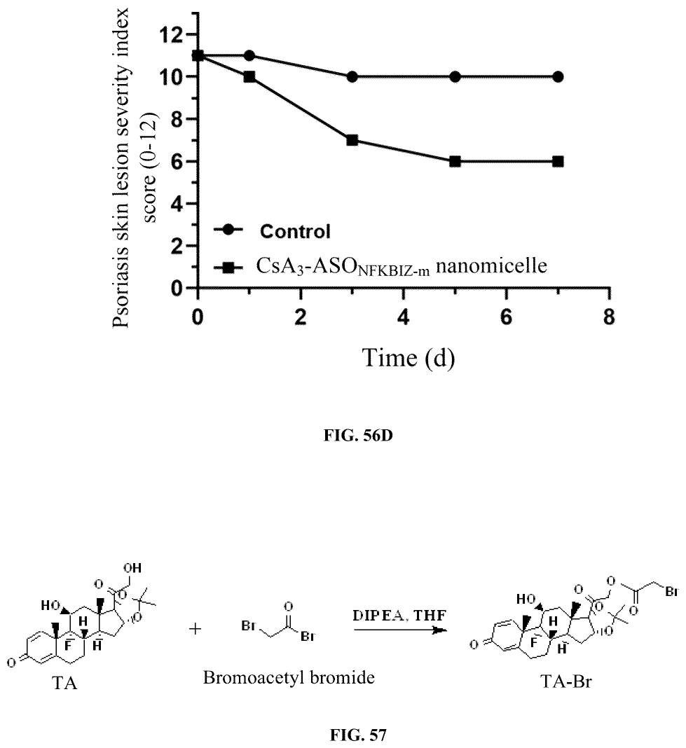

The psoriasis model on the back and ears of 6-8 weeks old mice (C57BL/6, Suzhou Xishan Biotechnology Co., Ltd.) was established with 5% imiquimod cream. After removing the hair on the back of the mice, 60 mg of 5% imiquimod cream was applied to the back and ears of each mouse for 6 consecutive days. 6 d after modeling, FIG. 53 showed that the mice developed obvious psoriasis-like phenotypes such as erythema, scaling, and obvious thickening of the skin on their backs and ears, indicating that the model was successfully constructed. CsA₃-ASO_{NFKBIZ-m} nanomicelles were mixed with carbomer gel and applied to the skin lesions for treatment. The carbomer gel without CsA₃-ASO_{NFKBIZ-m} nanomicelles was used as a control group (control) for efficacy testing. On the 7 d of treatment, the thickness of the skin at the back lesions and the thickness of the ears were measured, and the erythema, scaling, skin thickening, and severity of the psoriasis lesions were scored. The results were shown in FIG. 54, FIGS. 55A-55B, and FIGS. 56A-56D. After 7 d of treatment, compared to the control group, the erythema and scaling of the skin lesions in the CsA₃-ASO_{NFKBIZ-m} nanomicelle group were significantly reduced, and the skin thickness of the ears and lesions was reduced, indicating that CsA₃-ASO_{NFKBIZ-m} nanomicelles could effectively alleviate the psoriasis-like phenotype of diseased mice.

### Example 13 Synergistic Treatment of Psoriasis with TA-NFKBIZ ASO Covalent Conjugate (10TA-ASO_{NFKBIZ}) and its Nanomicelle Assembly

### 13.1 Synthesis of Carboethyl Bromo-Triamcinolone Acetonide (TA-Br)

The specific synthesis steps of the TA-Br were shown in FIG. 57. Under nitrogen protection, TA (200 mg) was dissolved in 50 mL of anhydrous DCM in a dry 250 mL round-bottom flask, 130 μL of DIPEA was added dropwise, and 105 mg of bromoacetyl bromide was added dropwise under ice bath conditions. The mixture was reacted by stirring at room temperature for 12 h. After the reaction was completed, the product was washed 3 times with 50 mL of saturated sodium bicarbonate solution and once with 50 mL of saturated sodium chloride solution. The organic phase was collected, dried over anhydrous sodium sulfate, and concentrated by vacuum distillation. The organic phase was separated by silica gel column chromatography, with an eluent of petroleum ether/ethyl acetate to obtain a light yellow solid (140 mg), namely the TA-Br.

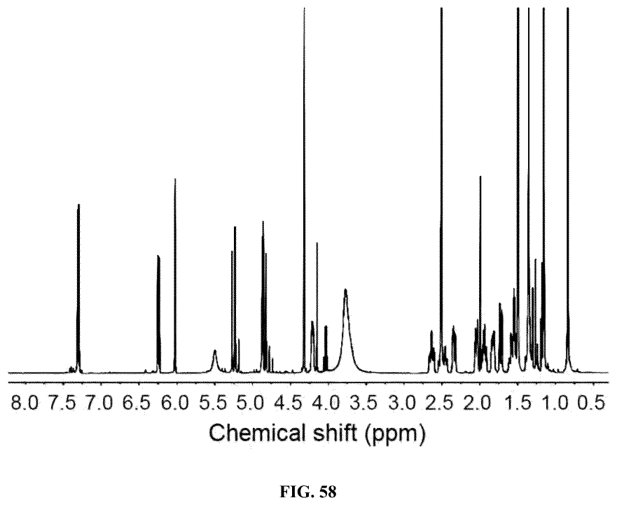

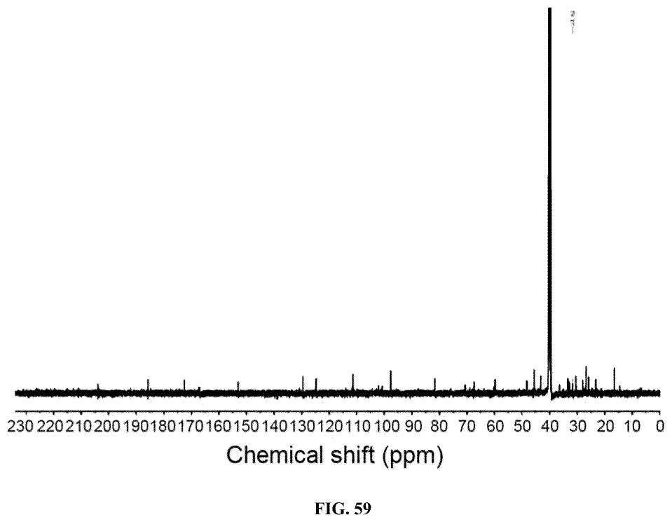

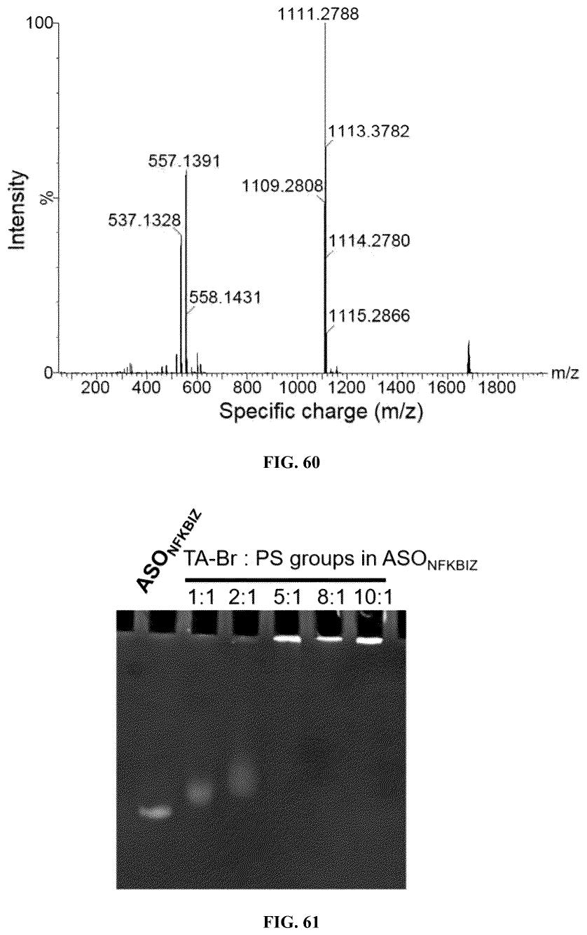

The ¹H NMR spectrum of TA-Br was shown in FIG. 58, the test solvent was DMSO-d6, and the attribution of each proton peak was as follows: δ (ppm): 7.30 (1H, d), 6.25 (1H, dd), 6.02 (1H, s), 5.50 (1H, s), 5.27 (1H, d), 4.86 (1H, d), 4.82 (1H, d), 4.32-4.15 (2H, d), 4.21 (1H, s), 3.78 (6H, s), 2.64 (1H, m), 2.34 (1H, dd), 2.05 (1H, m), 1.95 (1H,q), 1.82 (1H, t), 1.73 (1H, d), 1.55 (2H, m), 1.50 (3H, s), 1.35 (4H, s), 1.26 (1H, t), 1.16 (3H, s), 0.80 (3H, s). The ¹³C NMR spectrum of TA-Br was shown in FIG. 59, the test solvent was DMSO-d6, and the attribution of the characteristic carbon after coupling with carbonylethyl bromide was as follows: 210.50, 185.68, 167.04, 153.19, 129.78, 124.69, 110.83, 102.06, 97.28, 81.62, 70.66, 66.28, 48.43, 45.14, 42.95, 35.48, 33.47, 32.89, 30.57, 27.95, 26.77, 25.72, 23.26, 16.83. The theoretical molecular weight of TA-Br was 555.43, the m/z values measured by HPLC/quadrupole rod flight mass spectrometry were 557.1391 and 1,113.2782, which were attributed to the [M+1]⁺ and [2M+1]⁺ peaks of TA-Br, confirming that the target product was successfully synthesized, as shown in FIG. 60.

### 13.2 Synthesis of Triamcinolone Acetonide-NFKBIZ ASO Conjugate (10TA-ASO_{NFKBIZ})

In this example, the ASO sequences used to target NFKBIZ gene were purchased from Shanghai Sangon Biotech Co., Ltd. The phosphate backbone of the ASO was modified with thio (PS) to enable undergo substitution with a carbonylethyl bromide group modified on the TA-Br for efficient coupling. The sequence was as follows:

the sequence of the ASO targeting human NFKBIZ gene (ASO_{NFKBIZ-h}) was:

Human:
(SEQ ID NO: 67)
5′-ATCAGACAACGAATCGGGCTTTT*T*T*T*T*T*T*T*
T*T*T-3′;

a sequence of the ASO targeting mouse NFKBIZ gene (ASO_{NFKBIZ-m}) was:

Mouse:
(SEQ ID NO: 68)
5′-AATACTGGTACATTGACGCCTTTT*T*T*T*T*T*T*
T*T*T*T-3′.

The synthesis method of 10TA-ASO_{NFKBIZ} covalent conjugate was as follows: the TA-Br compound was dissolved in 20 μL of DMSO solution, added with 2 μL of thiophosphate group-modified ASO solution, where the ASO concentration was 200 μM (a ratio of thiophosphate group to TA-Br was 1:1 to 1:50), and a reaction was conducted by shaking at 55° C. overnight. After the reaction, 500 μL of ultrapure water was added, and the excess TA-Br in the reaction was removed by multiple extractions with ethyl acetate. The solvent was removed by vacuum distillation, and the mixture was redissolved in ultrapure water to obtain the TA-ASO conjugate. 15% denatured polyacrylamide gel electrophoresis was conducted to verify that the TA-Br was successfully grafted to the phosphorothioate-modified ASO, as shown in FIG. 61.

### 13.3 Preparation and Characterization of 10TA-ASO_{NFKBIZ} Nanomicelles

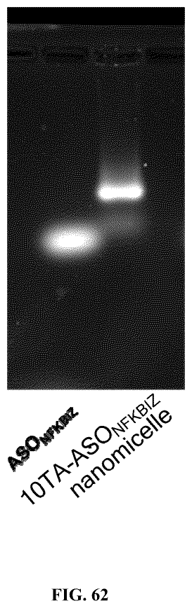

The 10TA-ASO_{NFKBIZ} covalent conjugate (10 OD ASO) was dissolved in 100 μL of DMSO and added dropwise to 300 μL of 1×PBS buffer with continuous stirring. Then, the mixture was placed in a dialysis bag and dialyzed in PBS overnight to remove DMSO, thereby obtaining the 10TA-ASO_{NFKBIZ} nanomicelle structure. The successful preparation of the 10TA-ASO_{NFKBIZ} nanomicelles was characterized by 1% agarose gel electrophoresis, as shown in FIG. 62.

### 13.4 Gene Regulation of 10TA-ASO_{NFKBIZ} Nanomicelles

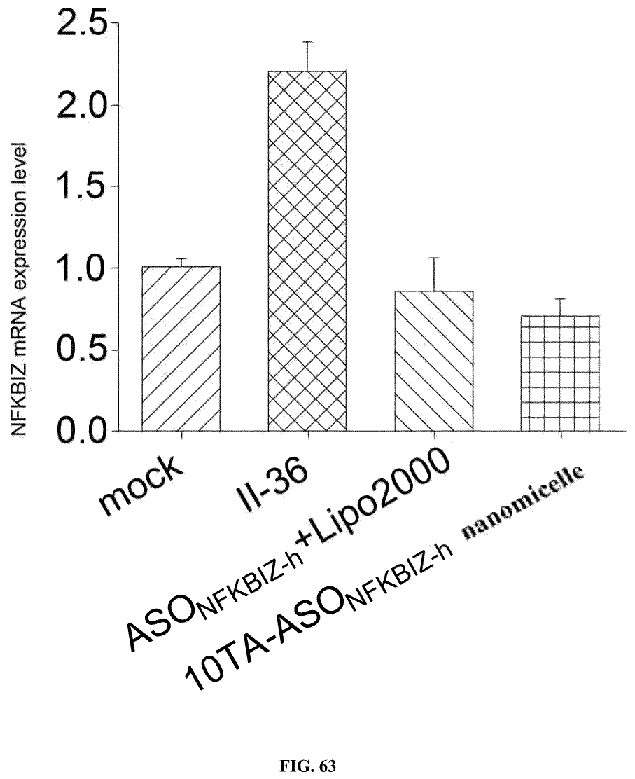

HaCaT cells from human keratinocyte cell line were inoculated in a 6-well plate at a density of 1×10⁵ cells/well and cultured until adherence. The cells were then stimulated with 100 ng/ml interleukin-36 (IL-36) for 2 h. After washing with 1×PBS buffer, 1 mL of Opti-MEM medium containing ASO_{NFKBIZ-h}+Lipo2000 complex and 10TA-ASO_{NFKBIZ-h} nanomicelles (all containing 10 μM ASO_{NFKBIZ-h}) were added, respectively. After incubation with HaCaT cells for 6 h, the cells were transferred into fresh DMEM medium, while cells without any treatment or stimulated with IL-36 alone were used as a control, all cells were incubated at 37° C. for 48 h. The cells were collected and RNA was extracted for RT-qPCR to analyze the expression level of the NFKBIZ mRNA in HaCaT cells, and experimental results were shown in FIG. 63.

As shown in FIG. 63, after IL-36 stimulation, the NFKBIZ mRNA expression of HaCaT cells increased to about 2.2 times that of normal cells. ASO_{NFKBIZ-h} after Lipo2000 transfection could downregulate it to about 0.85 times. 10TA-ASO_{NFKBIZ-h} nanomicelles downregulated the NFKBIZ gene to about 0.7 times. Therefore, compared to single ASO_{NFKBIZ-h}, 10TA-ASO_{NFKBIZ-h} nanomicelles had a better ability to downregulate the expression of NFKBIZ gene.

### 13.5 Therapeutic Effect of 10TA-ASO_{NFKBIZ-m} Nanomicelles on Psoriasis

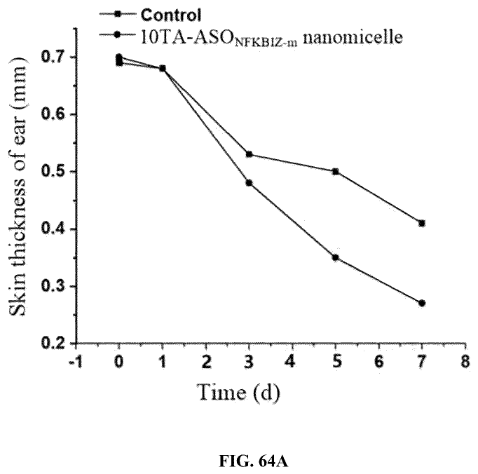

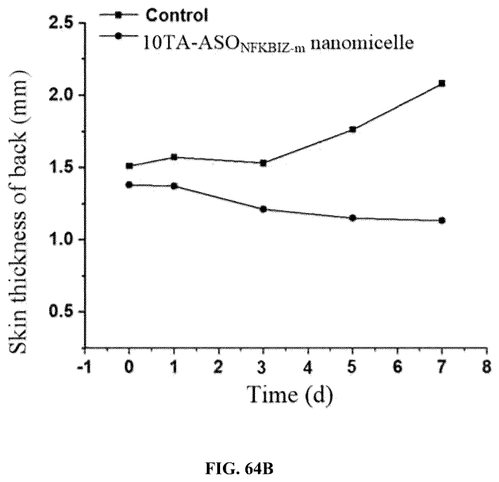

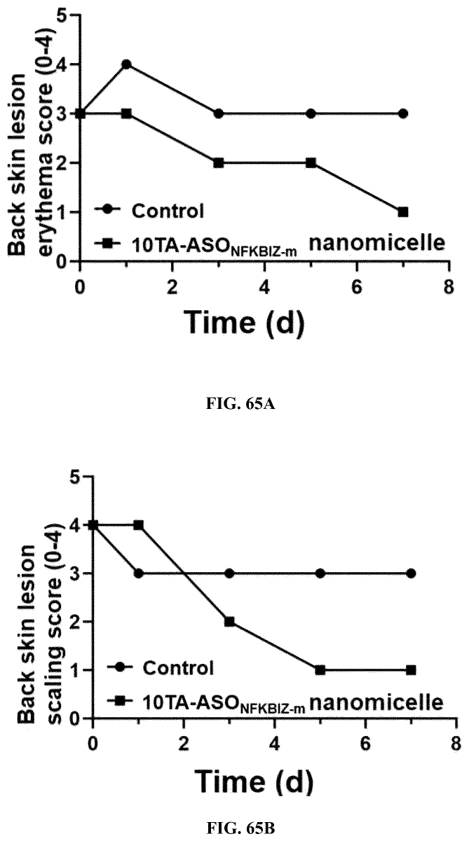

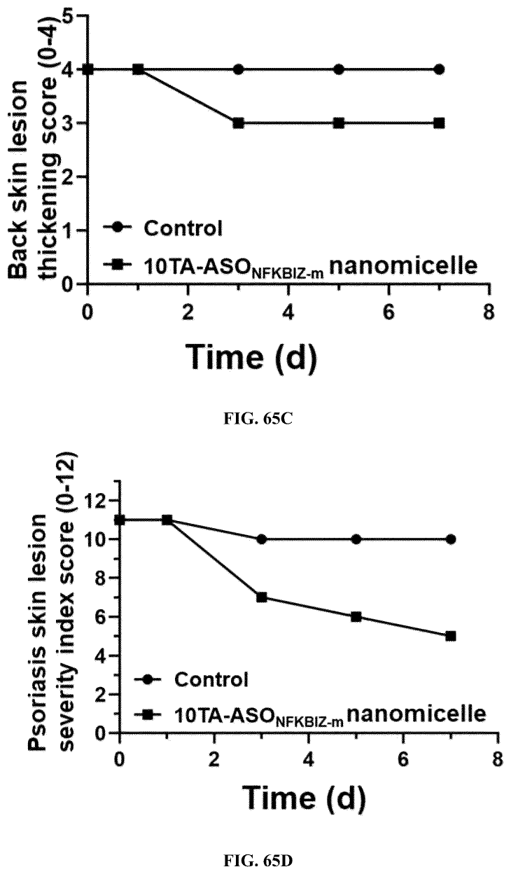

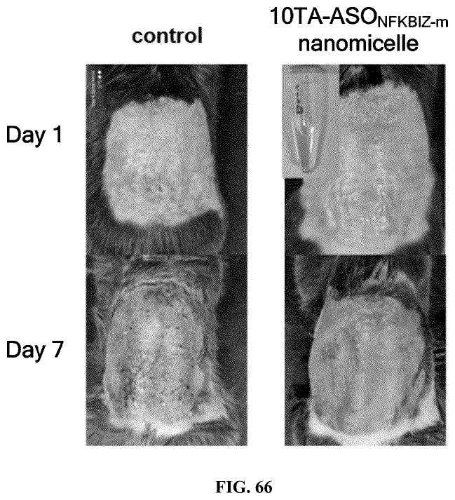

The psoriasis model on the back and ears of 6-8 weeks old mice was established with 5% imiquimod cream. After removing the hair on the back of the mice, 60 mg of 5% imiquimod cream was applied to the back and ears of each mouse for 6 consecutive days. 6 d after modeling, the mice developed obvious psoriasis-like phenotypes such as erythema, scaling, and obvious thickening of the skin on their backs and ears, indicating that the model was successfully constructed. 10TA-ASO_{NFKBIZ}-m nanomicelles were mixed with carbomer gel and applied to the skin lesions for treatment. The carbomer gel without 10TA-ASO_{NFKBIZ-m} nanomicelles was used as a control group (control) for efficacy testing. On the 7 d of treatment, the thickness of the skin at the back lesions and the thickness of the ears were measured, and the erythema, scaling, skin thickening, and severity of the psoriasis lesions were scored. The experimental results were shown in FIGS. 64A-64B, FIGS. 65A-65D, and FIG. 66. After 7 d of treatment, compared with the untreated group, the erythema and scaling of the skin lesions in the 10TA-ASO_{NFKBIZ-m} nanomicelle group were significantly reduced, and the skin thickness of the lesions was reduced, indicating that 10TA-ASO_{NFKBIZ-m} nanomicelles could effectively alleviate the psoriasis-like phenotype of diseased mice.

### Example 14 Synergistic Treatment of Psoriasis with Calcipotriol Molecule-NFKBIZ ASO Covalent Conjugate (10Cal-ASO_{NFKBIZ}) and its nanomicelle assembly

### 14.1 Synthesis of Benzyl Bromide-Modified Calcipotriol (Cal-Bz-Br)

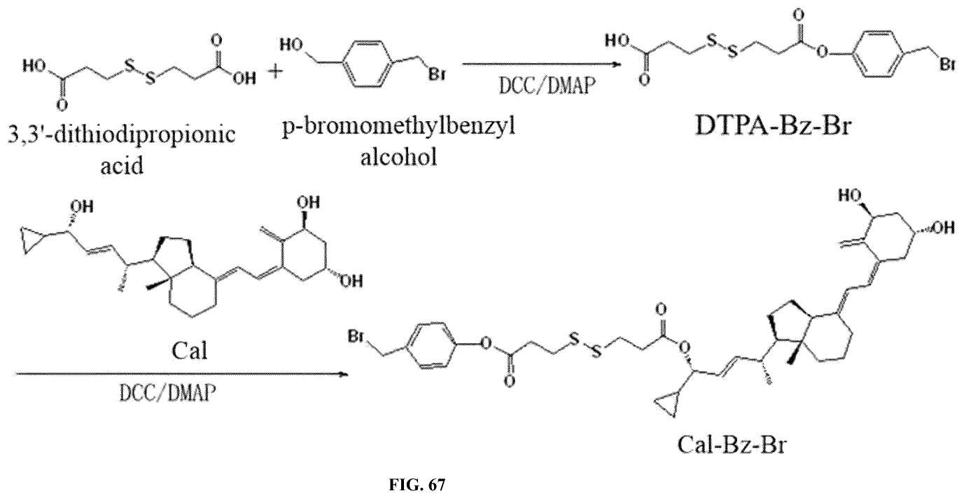

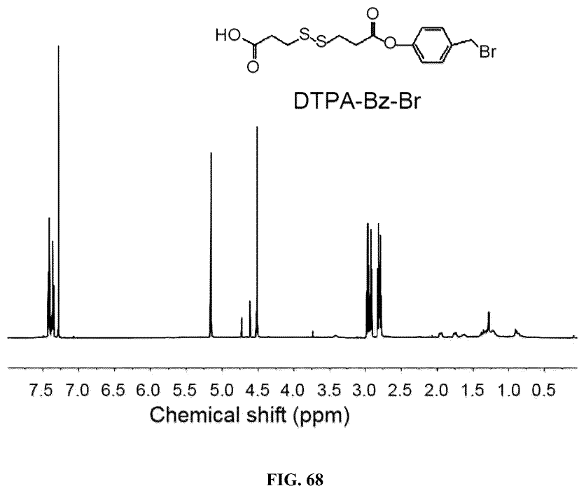

The synthesis steps of Cal-Bz-Br were shown in FIG. 67. a) Synthesis of benzyl bromide-modified dithiodipropionic acid (DTPA-Bz-Br): 500 mg of 4-bromomethylbenzyl alcohol and 2.6 g of 3,3′-dithiodipropionic acid (DTPA) were dissolved in a mixed solution of ultra-dry DCM and tetrahydrofuran (THF)(1/1, v/v); 91 mg of 4-dimethylaminopyridine was added, and 615 mg of dicyclohexylcarbodiimide (dissolved in ultra-dry DCM) was added dropwise after stirring for 5 min. The mixture was reacted at room temperature overnight, and the solvent was evaporated to dryness by rotary evaporator. The benzyl bromide structure (DTPA-Bz-Br) containing a disulfide bond was separated by silica gel column chromatography, with an eluent of petroleum ether/ethyl acetate. The ¹H NMR and attribution of the product were shown in FIG. 68.

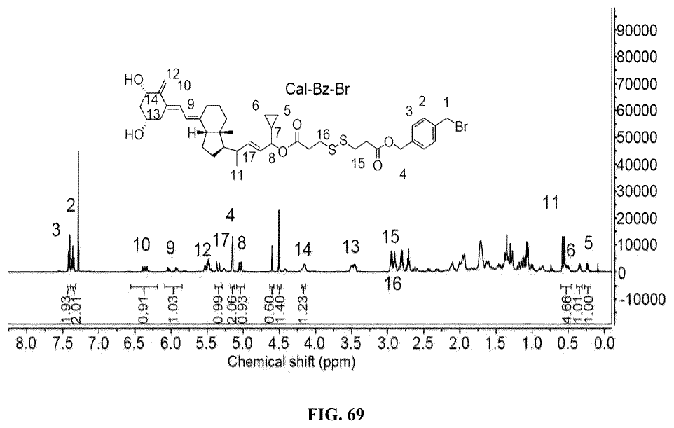

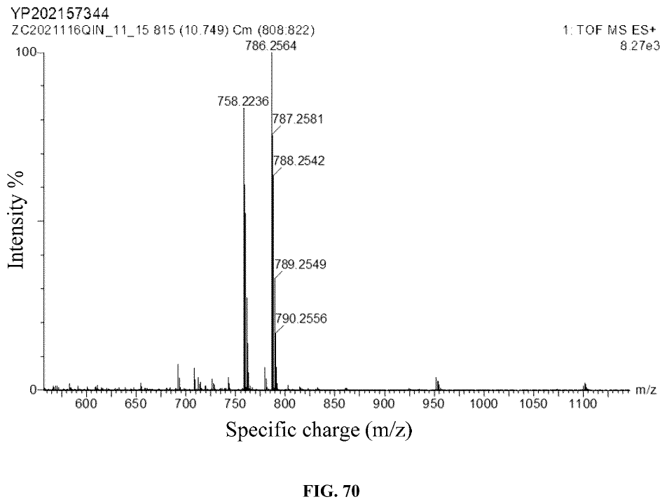

b) 135 mg of Cal and 81 mg of dicyclohexylcarbodiimide were added into 30 mL of anhydrous DCM and dissolved by stirring, 10 mg of 4-dimethylaminopyridine (dissolved in anhydrous DCM) was added, and 10 mg of DTPA-Bz-Br (dissolved in anhydrous DCM) was added dropwise. The mixture was stirred at room temperature for 3 h, the solvent was evaporated to dryness using a rotary evaporator, and the benzyl bromide-modified Cal prodrug molecule (Cal-Bz-Br) was separated by silica gel column chromatography, with an eluent of petroleum ether/ethyl acetate. The NMR of the product was shown in FIG. 69, the test solvent was DMSO-d6, and the attribution of each proton peak was as follows: δ (ppm): 7.43, H3 (2H, s), 7.36, H2 (2H, m), 6.35, H10 (1H, d), 6.02 H9 (1H,d), 5.52, H12 (2H,m), 5.35, H17 (1H, d), 5.14, H4 (2H,s), 5.06, H8 (1H, d), 4.51, H1 (2H, s), 4.15, H14 (1H, m), 3.48, H13 (1H, m), 2.94, H15 (2H, m), 2.91, H16 (2H, m), 0.58, H11 (3H, d), 0.52, H6 (2H,m), 0.33, H5 (1H, m), 0.25, H7 (1H, m). The theoretical molecular weight of Cal-Bz-Br was 785.2, the m/z value measured by HPLC/quadrupole rod flight mass spectrometry was 786.25, which were attributed to the [M+1]⁺ peaks of Cal-Bz-Br, confirming that the target product was successfully synthesized, as shown in FIG. 70.

### 14.2 Synthesis of Calcipotriol-NFKBIZ ASO Conjugate (10Cal-ASO_{NFKBIZ})

In this example, the ASO sequences used to target NFKBIZ gene were purchased from Shanghai Sangon Biotech Co., Ltd. The phosphate backbone of the ASO was modified with thio (PS) to enable undergo substitution with a benzyl bromide group modified on the Cal-Br for efficient coupling. The sequence was as follows:

the sequence of the ASO targeting human NFKBIZ gene (ASO_{NFKBIZ-h}) was:

Human:
(SEQ ID NO: 69)
5′-ATCAGACAACGAATCGGGCTTTT*T*T*T*T*T*T*
T*T*T*T-3′;

the sequence of the ASO targeting mouse NFKBIZ gene (ASO_{NFKBIZ-m}) was:

Mouse:
(SEQ ID NO: 70)
5′-AATACTGGTACATTGACGCCTTTT*T*T*T*T*T*T*
T*T*T*T-3′.

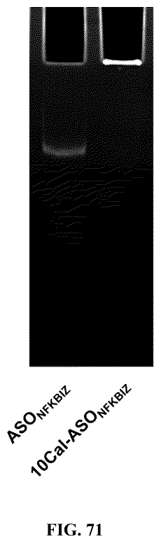

The synthesis method of calcipotriol-NFKBIZ ASO conjugate (10Cal-ASO_{NFKBIZ}) was as follows: the Cal-Bz-Br compound was dissolved in 20 μL of DMSO solution, added with 2 μL of thiophosphate group-modified ASO solution, where the ASO concentration was 200 μM (a ratio of thiophosphate group to Cal-Bz-Br was 1:5), and a reaction was conducted by shaking at 55° C. overnight. After the reaction, 500 μL of ultrapure water was added, and the excess Cal-Bz-Br in the reaction was removed by multiple extractions with ethyl acetate. The solvent was removed by vacuum distillation to obtain the calcipotriol-ASO conjugate prodrug. 15% denatured polyacrylamide gel electrophoresis (PAGE) was conducted to verify that the Cal was successfully grafted to the phosphorothioate-modified ASO, as shown in FIG. 71.

### 14.3 Preparation and Characterization of 10Cal-ASO_{NFKBIZ} Nanomicelles

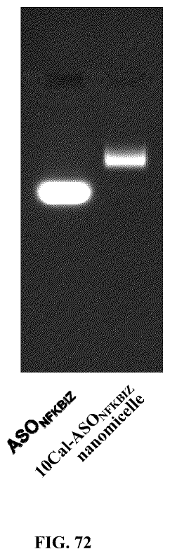

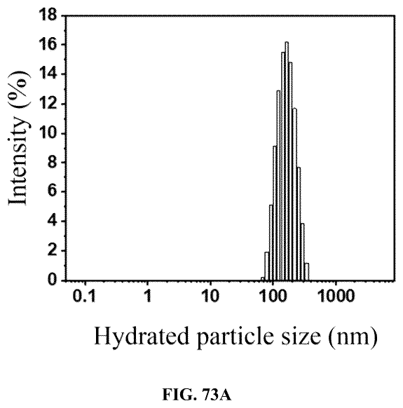

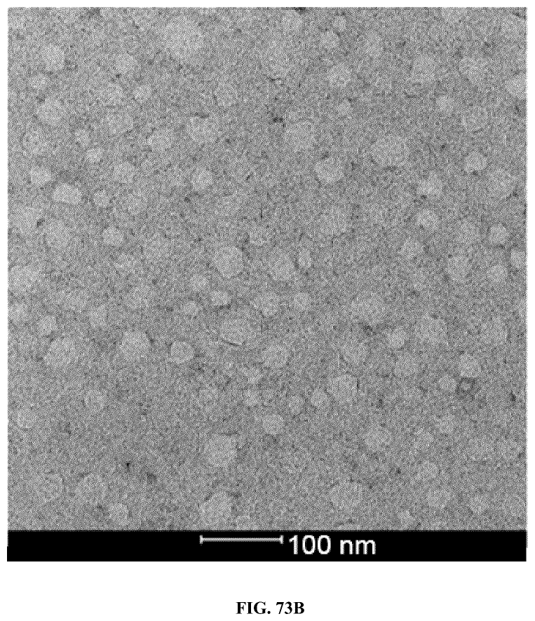

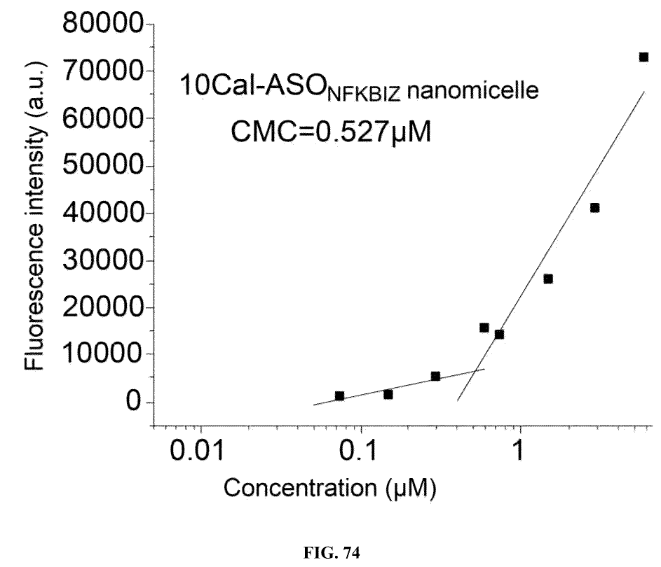

The 10Cal-ASO_{NFKBIZ} covalent conjugate (10 OD ASO) was dissolved in 100 μL of DMSO and added dropwise to 300 μL of 1×PBS buffer with continuous stirring. Then, the mixture was placed in a dialysis bag and dialyzed in PBS overnight to remove DMSO, thereby obtaining the 10Cal-ASO_{NFKBIZ} nanomicelle structure. The successful assembly was confirmed by 1% agarose gel electrophoresis, and the results were shown in FIG. 72. The hydrated particle size of the Cal-ASO conjugate self-assembled nanomicelles was measured by DLS. As shown in FIGS. 73A-73B, the hydrated particle size of the Cal-ASO conjugate self-assembled nanomicelles was about 150 nm. The TEM image in FIGS. 73A-73B revealed that the prepared nanomicelles had a spherical morphology. The critical micelle concentration of the 10Cal-ASO_{NFKBIZ} conjugate was determined to be 0.527 μM, as shown in FIG. 74.

### 14.4 Gene Regulation of 10Cal-ASO_{NFKBIZ} Nanomicelles

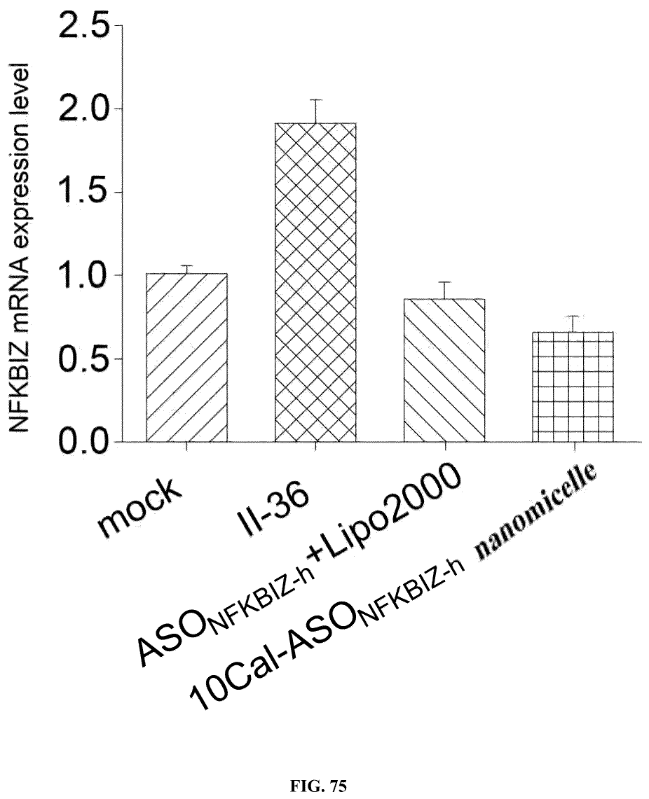

HaCaT cells from human keratinocyte cell line were inoculated in a 6-well plate at a density of 1×10⁵ cells/well and cultured until adherence. The cells were then stimulated with 100 ng/ml interleukin-36 (IL-36) for 2 h. After washing with 1×PBS buffer, 1 mL of Opti-MEM medium containing ASO_{NFKBIZ-h}+Lipo2000 complex and 10Cal-ASO_{NFKBIZ-h} nanomicelles (all containing 10 μM ASO_{NFKBIZ-h}) were added, respectively. After incubation with HaCaT cells for 6 h, the cells were transferred into fresh DMEM medium, while cells without any treatment or stimulated with IL-36 alone were used as a control, all cells were incubated at 37° C. for 48 h. The cells were collected and RNA was extracted for RT-qPCR to analyze the expression level of the NFKBIZ mRNA in HaCaT cells, and experimental results were shown in FIG. 75.

After IL-36 stimulation, the NFKBIZ mRNA expression of HaCaT cells increased to about 1.9 times that of normal cells. ASO_{NFKBIZ-h} after Lipo2000 transfection could downregulate it to about 0.85 times. 10Cal-ASO_{NFKBIZ-h} nanomicelles downregulated the NFKBIZ gene to about 0.65 times. Therefore, compared to the single ASO_{NFKBIZ-h}, 10Cal-ASO_{NFKBIZ-h} nanomicelles had a better ability to downregulate the expression of NFKBIZ gene.

### 14.5 Therapeutic Effect of 10Cal-ASO_{NFKBIZ-m} Nanomicelles on Psoriasis

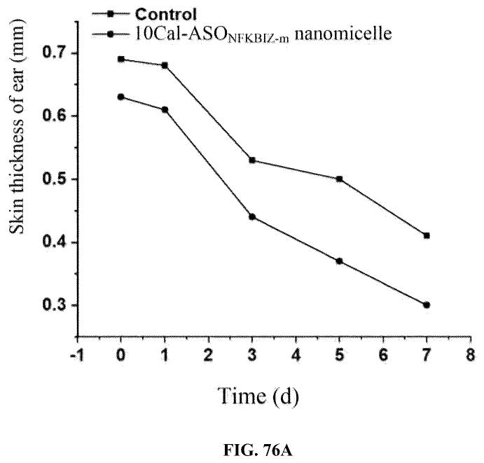

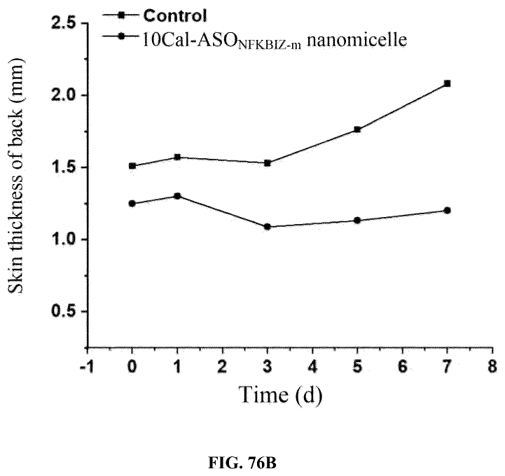

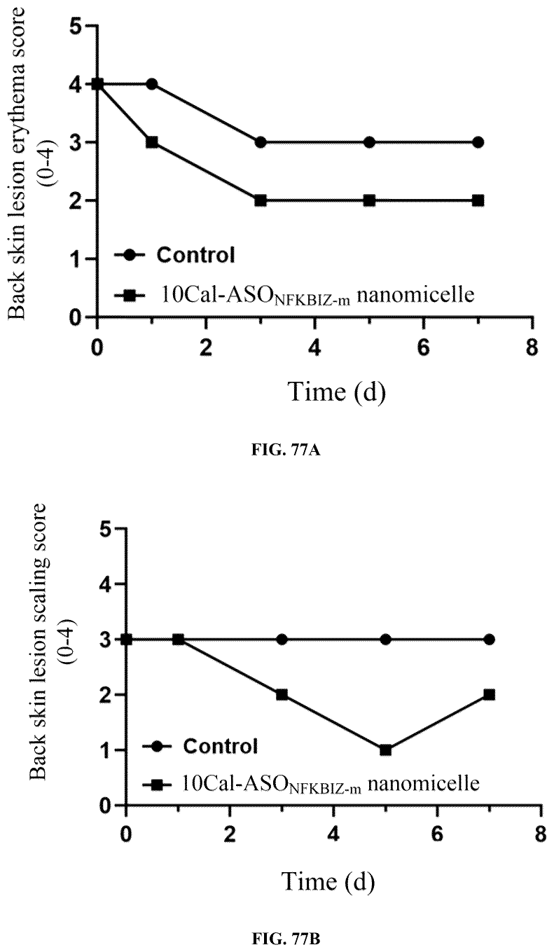

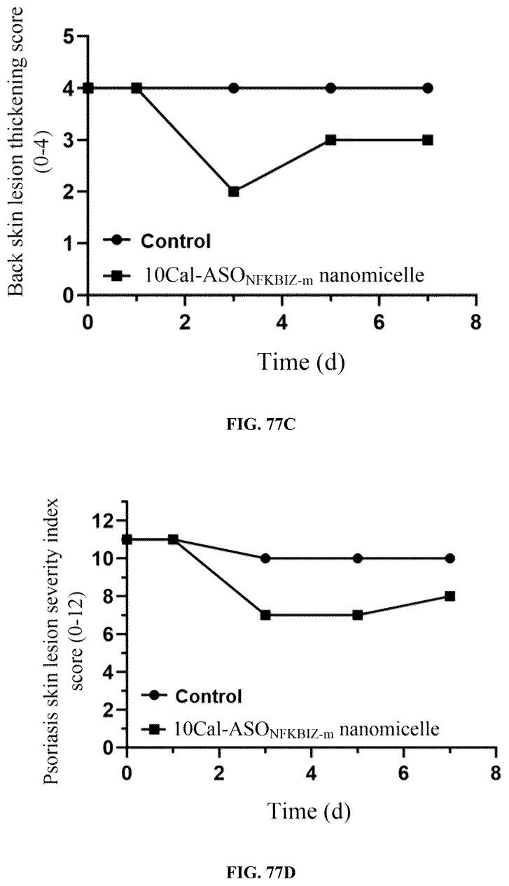

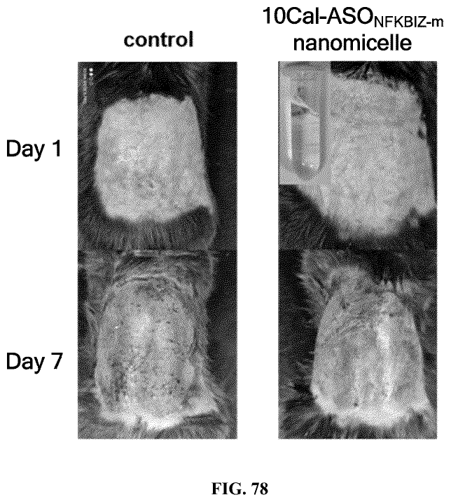

The psoriasis model on the back and ears of 6-8 weeks old mice was established with 5% imiquimod cream (Sichuan Med-Shine Pharmaceutical Co., Ltd.). After removing the hair on the back of the mice, 60 mg of 5% imiquimod cream was applied to the back and ears of each mouse for 6 consecutive days. 6 d after modeling, the mice developed obvious psoriasis-like phenotypes such as erythema, scaling, and obvious thickening of the skin on their backs and ears, indicating that the model was successfully constructed. 10Cal-ASO_{NFKBIZ-m} nanomicelles were mixed with carbomer gel and applied to the skin lesions for treatment. The carbomer gel without 10Cal-ASO_{NFKBIZ-m} nanomicelles was used as a control group (control) for efficacy testing. On the 7 d of treatment, the thickness of the skin at the back lesions and the thickness of the ears were measured, and the erythema, scaling, skin thickening, and severity of the psoriasis lesions were scored. The experimental results were shown in FIGS. 76A-76B, FIGS. 77A-77D, and FIG. 78. After 7 d of treatment, compared to the untreated group, the erythema and scaling of the skin lesions in the 10Cal-ASO_{NFKBIZ-m} nanomicelle group were significantly reduced, and the skin thickness of the lesions was reduced, indicating that 10Cal-ASO_{NFKBIZ-m} nanomicelles could effectively alleviate the psoriasis-like phenotype of diseased mice.

Although the above example has described the present disclosure in detail, they are only a part of, not all of, the examples of the present disclosure. Other examples may also be obtained by persons based on the examples without creative efforts, and all of these examples shall fall within the protection scope of the present disclosure.

1. A small molecule drug-oligonucleotide conjugate, wherein the small molecule drug-oligonucleotide conjugate is prepared by covalent coupling of a small molecule drug with an immunomodulatory function and a functional oligonucleotide molecule capable of regulating expression of an inflammation-related gene.

2. The small molecule drug-oligonucleotide conjugate according to claim 1, wherein the covalent coupling of the small molecule drug and the functional oligonucleotide molecule is implemented through a chemical linker.

3. The small molecule drug-oligonucleotide conjugate according to claim 1, wherein the small molecule drug is a small molecule drug that acts on an immune-related signaling pathway and is capable of regulating an immune response; and wherein the small molecule drug is one or more selected from the group consisting of a calcineurin inhibitor, a glucocorticoid, a mammalian target of rapamycin (mTOR) inhibitor, and a vitamin D analog.

4. The small molecule drug-oligonucleotide conjugate according to claim 1, wherein the inflammation-related gene is one or more selected from the group consisting of a tumor necrosis factor-a gene, an interleukin 1b gene, an interleukin 17 gene, an interleukin 23 gene, an NFKBIZ gene, an inflammasome NLRP3 gene, a JAK gene, and a PDE4 gene.

5. The small molecule drug-oligonucleotide conjugate according to claim 1, wherein the functional oligonucleotide molecule is one selected from the group consisting of a double-stranded small interfering RNA (siRNA), a microRNA (miRNA), and a single-stranded antisense oligonucleotide (ASO).

6. The small molecule drug-oligonucleotide conjugate according to claim 5, wherein when the functional oligonucleotide molecule is the double-stranded siRNA or the miRNA, the small molecule drug is covalently coupled to a 3′ end of a sense strand of the functional oligonucleotide molecule; and when the functional oligonucleotide molecule is the single-stranded ASO, the small molecule drug is covalently coupled to a 3′ end or a 5′ end of the single-stranded ASO.

7. The small molecule drug-oligonucleotide conjugate according to claim 6, wherein the number of the small molecule drugs covalently coupled to each functional oligonucleotide molecule is in a range of 1 to 40.

8. The small molecule drug-oligonucleotide conjugate according to claim 7, wherein the small molecule drug is covalently coupled to a terminal of the functional oligonucleotide molecule, or the small molecule drug is covalently coupled to a side chain base or a phosphate backbone of an extended sequence at a 3′ end or a 5′ end of the functional oligonucleotide molecule. 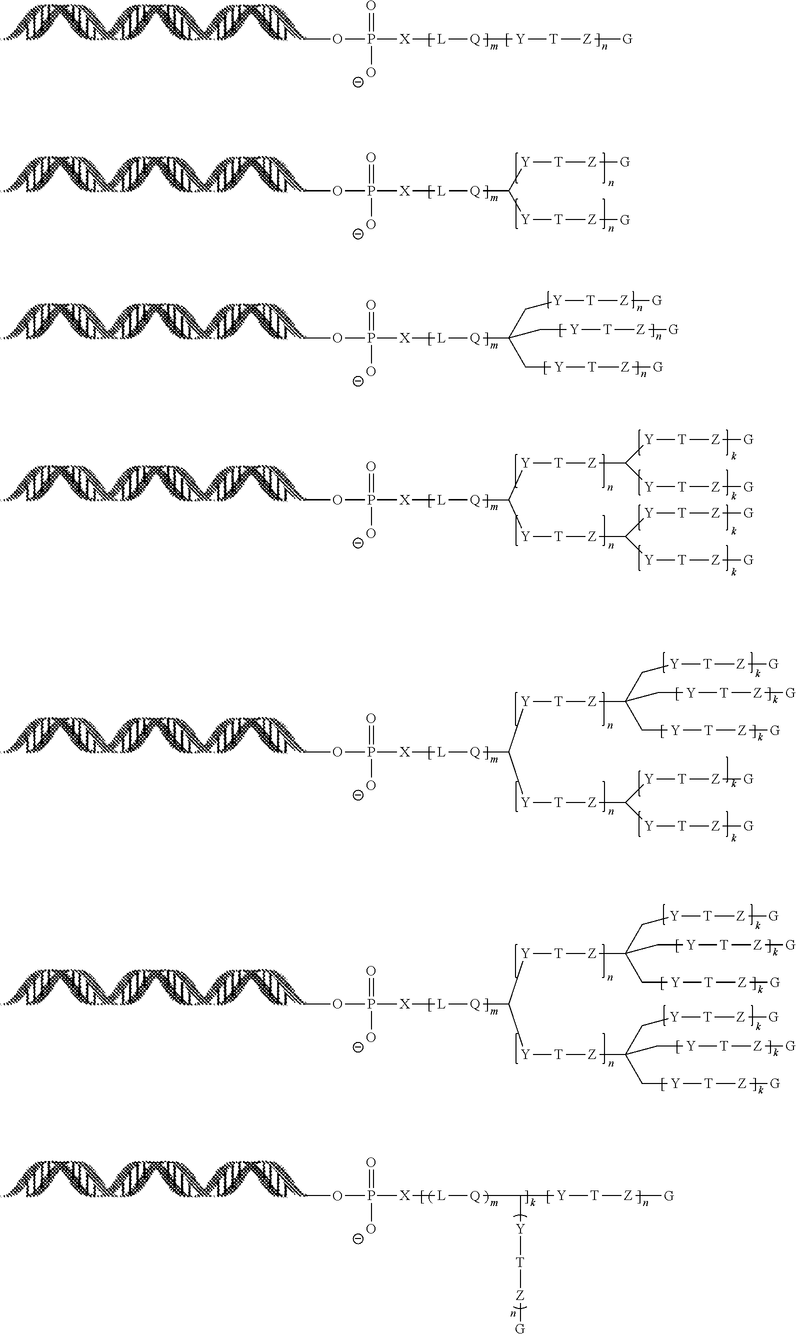 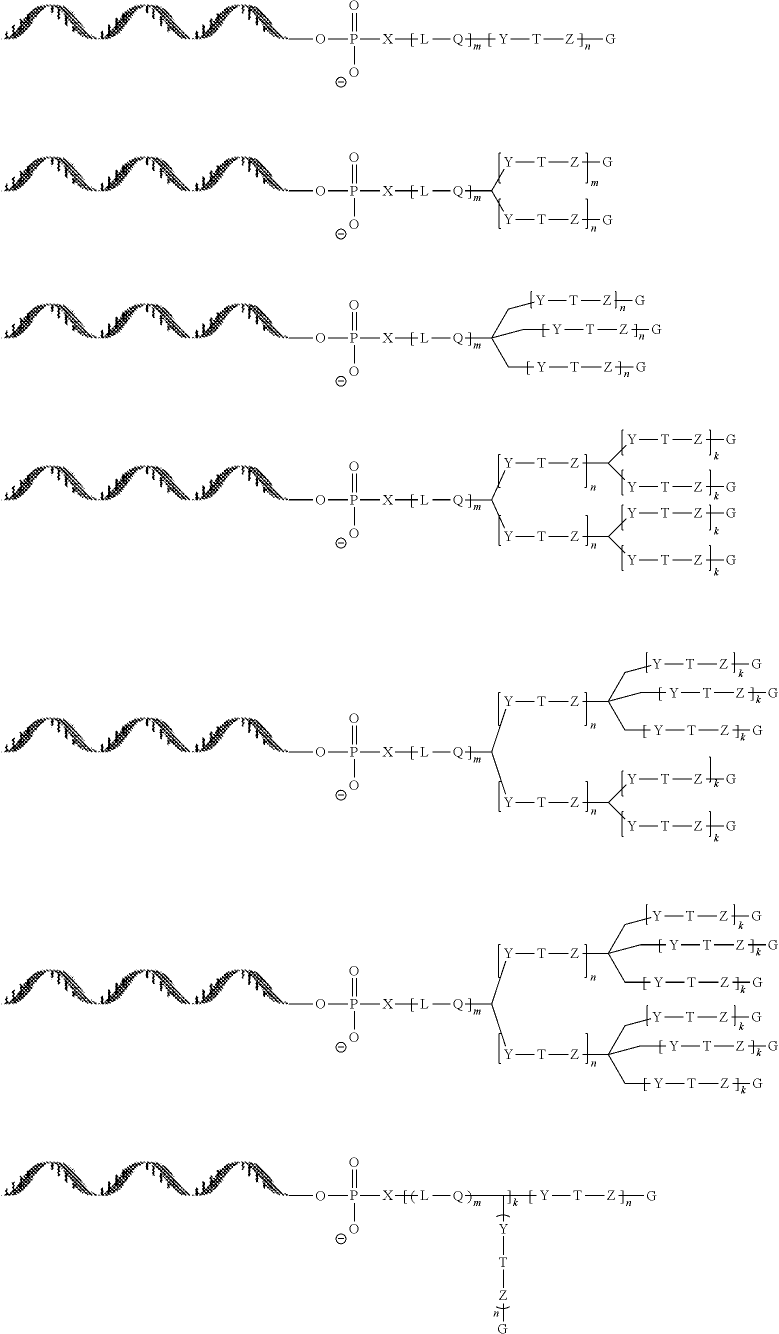 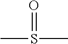 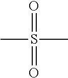 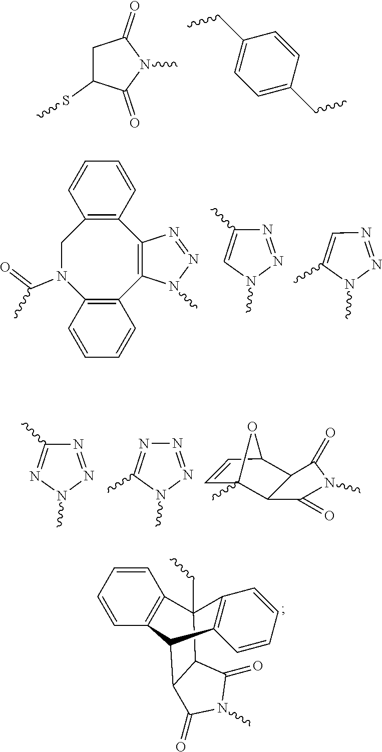 [symbol] 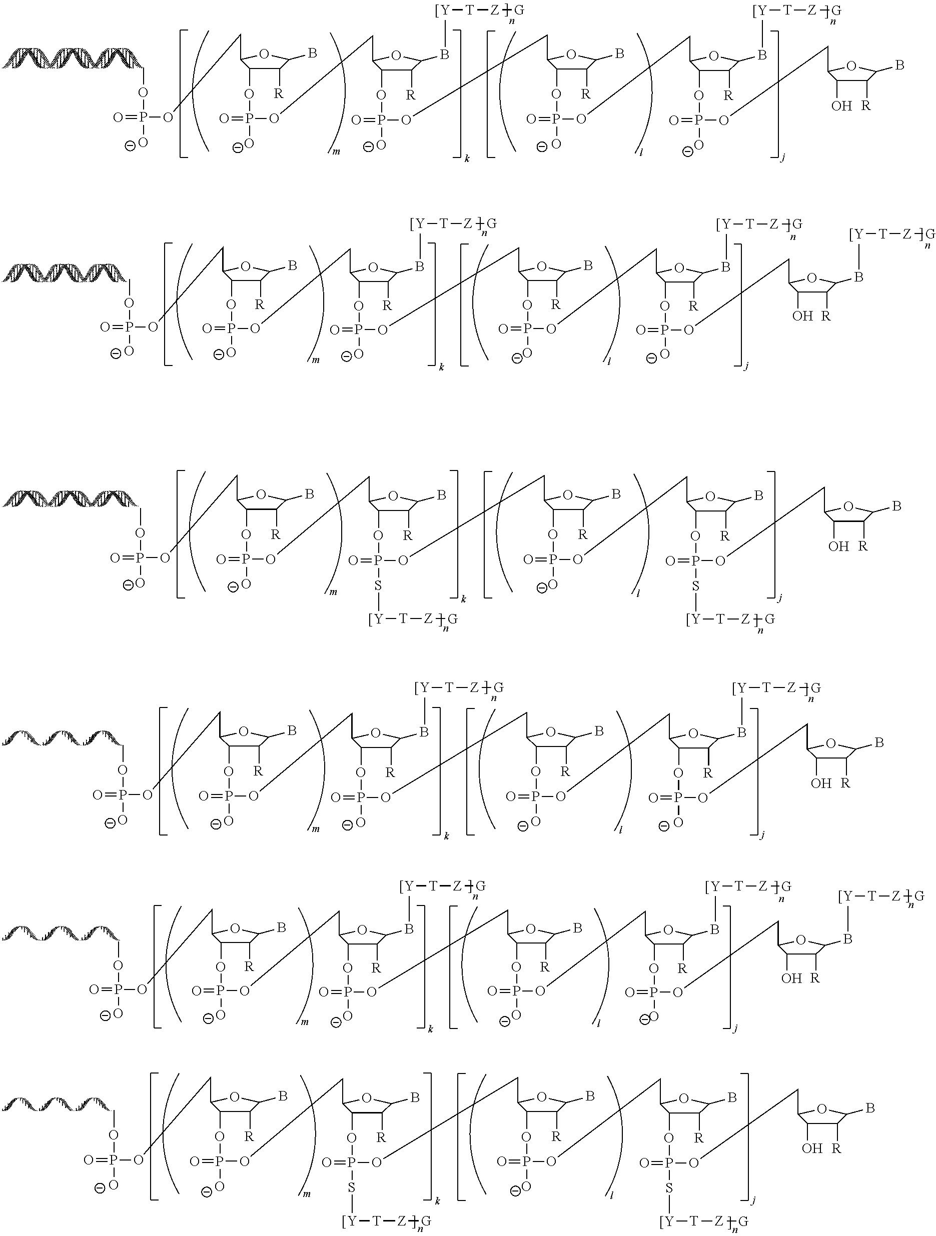 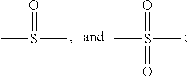 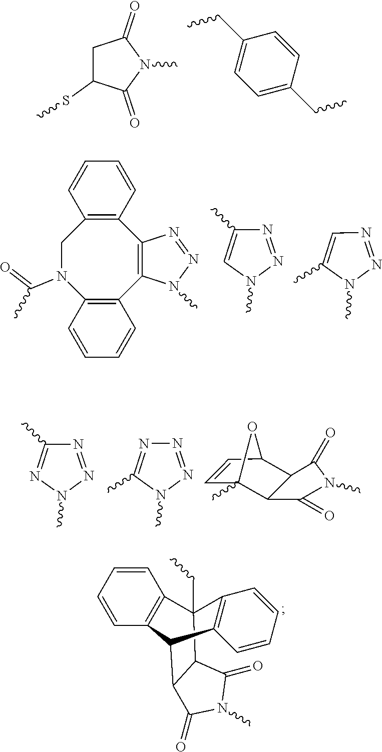 [symbol]
.

9. A method for treating an inflammation-related disease, comprising administering the small molecule drug-oligonucleotide conjugate to a subject in need thereof according to claim 1.

10. The method according to claim 9, wherein the inflammation-related disease is one or more selected from the group consisting of xerophthalmia, psoriasis, Sjögren's syndrome, uveitis, keratitis, conjunctivitis, atopic dermatitis, rheumatoid arthritis, inflammatory bowel disease, and Crohn's disease.

11. (canceled)

12. The small molecule drug-oligonucleotide conjugate according to claim 8, wherein when the small molecule drug is covalently coupled to the terminal of the functional oligonucleotide molecule, the small molecule drug-oligonucleotide conjugate has a chemical structure shown in Formula 1 to Formula 14: 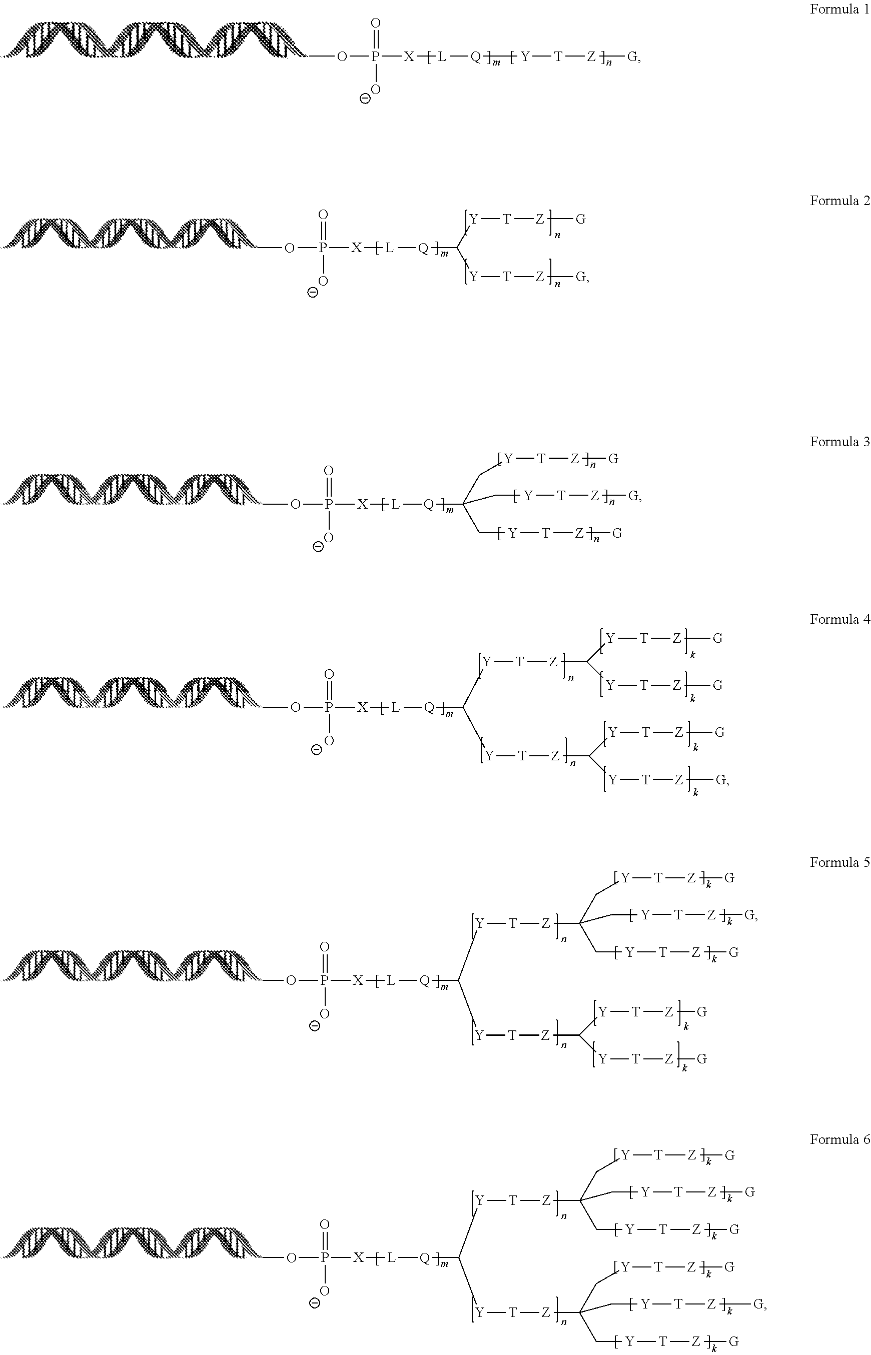 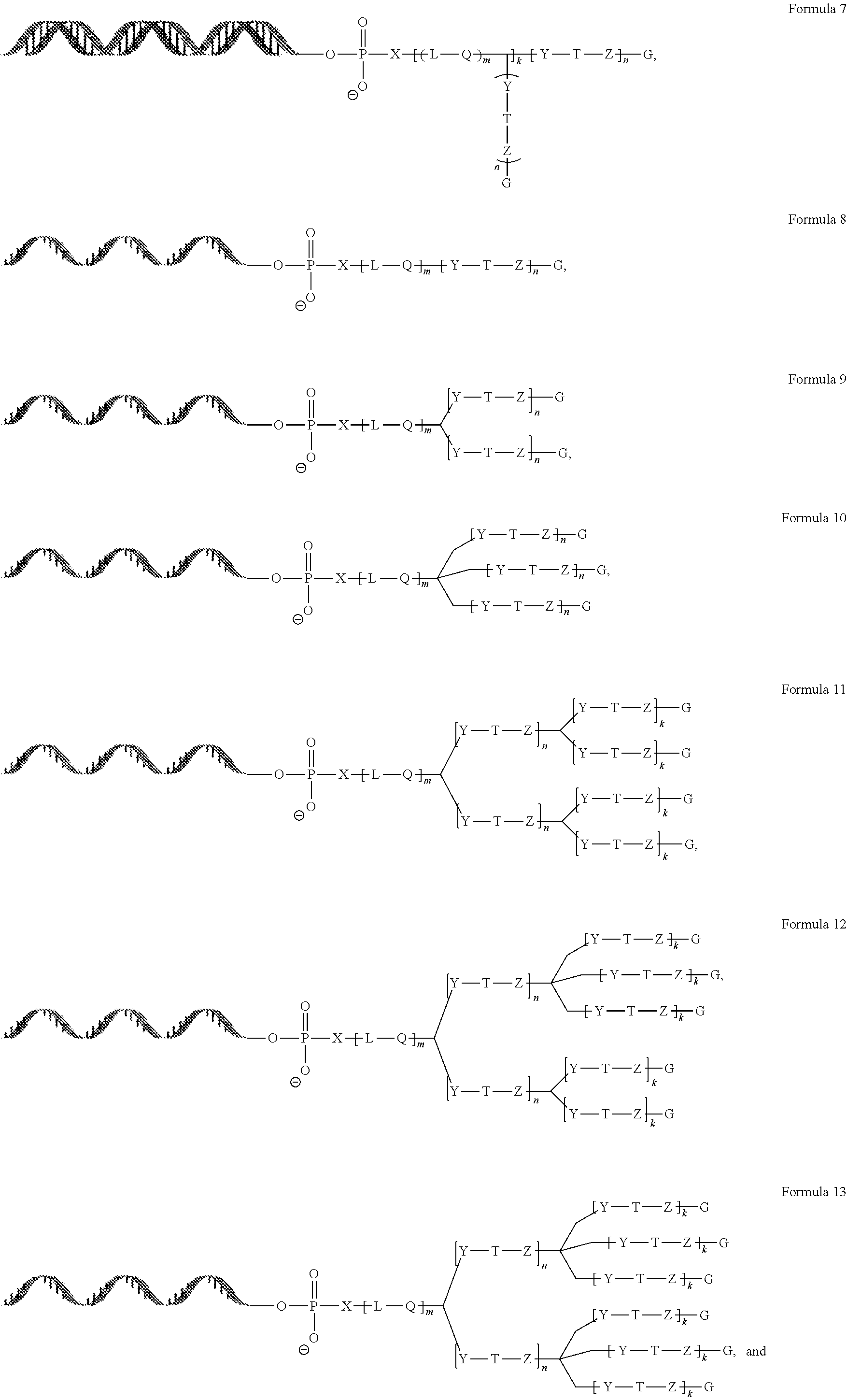 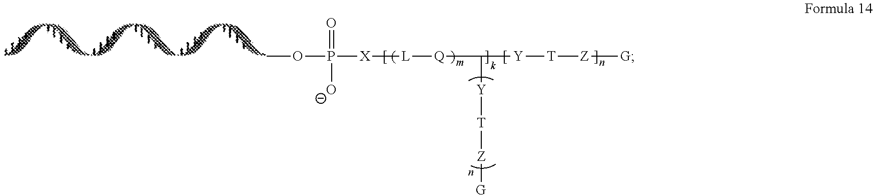
wherein
in Formula 1 to Formula 14, L and T are independently absent, or selected from the group consisting of —(CH₂)h- and a group formed by substituting any one or more alkylene groups in —(CH₂)h- with a group A; h is 0 to 15; the group A is selected from the group consisting of —O—, —S—, —C(O)—, —C(O)O—, —C(O)NH—, —CH(R^{C})—, —C(R′)(R″)—, —NH—, —N(R^{N})—, —S—S—, —C(R′)═C(R″)—, —C═C— 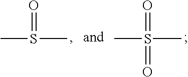
R^{C}, R^{N}, R′, and R″ in the group A represent that any one or more hydrogen atoms on a designated atom are substituted by a group B on condition that a normal valence of the designated atom is not exceeded and a stable compound is generated by substitution, and the designated atom is selected from the group consisting of a carbon atom and a nitrogen atom; the group B is selected from the group consisting of C1-C6 alkyl, C2-C6 alkenyl, C2-C6 alkynyl, cyano, hydroxyl, oxo, carboxyl, cycloalkyl, cycloalkenyl, heterocyclyl, heteroaryl, aryl, keto, alkoxycarbonyl, aryloxycarbonyl, heteroaryloxycarbonyl, and a halogen; the halogen is selected from the group consisting of F, Cl, Br, and I; and (O) in the group A represents a carbonyl oxygen atom;
in Formula 1 to Formula 14, Q, Y, and Z are independently absent, or selected from the group consisting of —O—, —S—, —C(O)—, —NH—, —CH₂—, —C(O)NH—, —NHC(O)—, —C(O)O—, —OC(O)—, —OC(O)O—, —OC(O)NH—, —NHC(O)O—, and 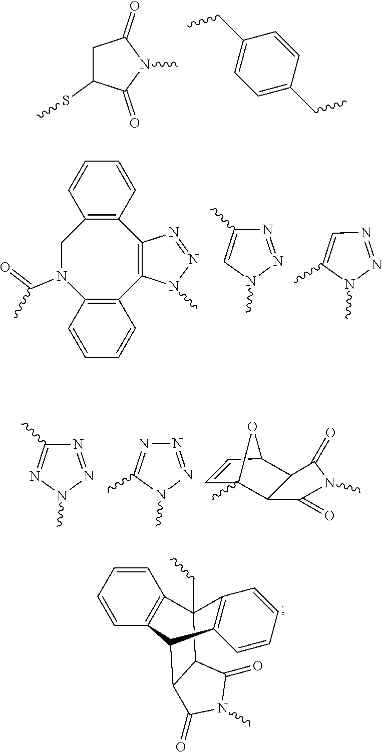
(O) In the Groups Q, Y, and Z Represents a Carbonyl Oxygen Atom; and [symbol] Represents a Ligation Site;
in Formula 1 to Formula 14, G represents a small molecule immunomodulatory drug; m, n, and k are independently 1 to 15; and X represents O or S.

13. The small molecule drug-oligonucleotide conjugate according to claim 8, wherein, when the small molecule drug is covalently coupled to the side chain base or the phosphate backbone of the extended sequence at the 3′ end or the 5′ end of the functional oligonucleotide molecule, the small molecule drug-oligonucleotide conjugate has a chemical structure shown in Formula 15 to Formula 20: 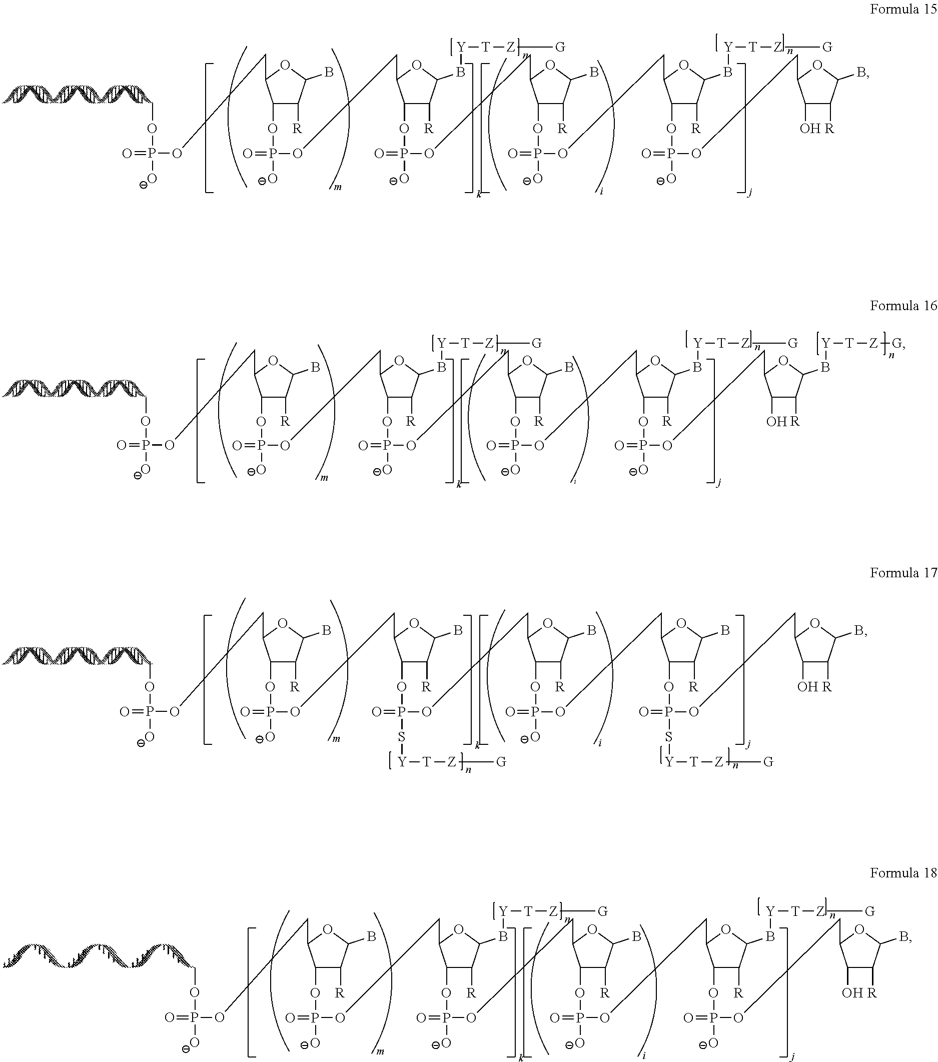 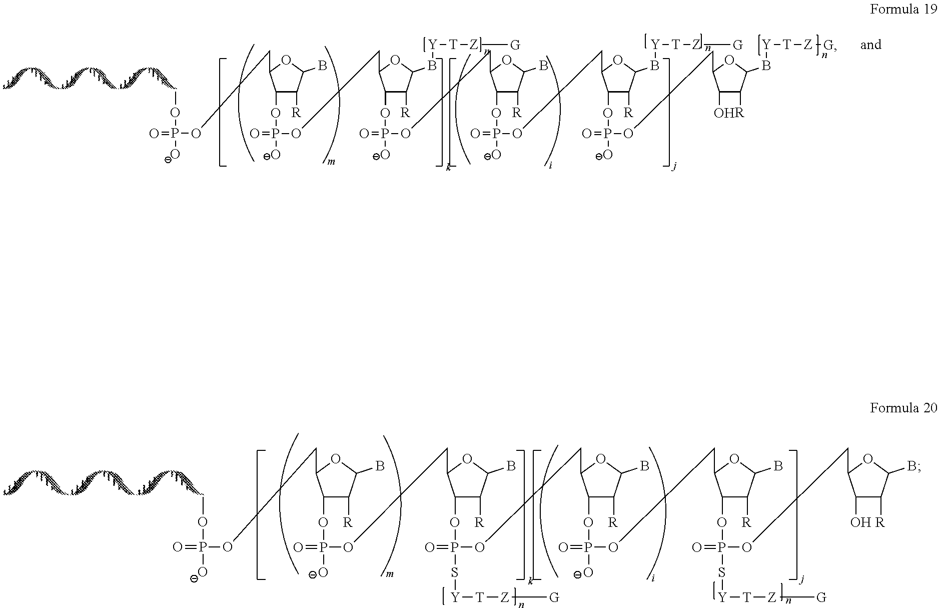
wherein
in Formula 15 to Formula 20, T is absent, or selected from the group consisting of —(CH₂)h- and a group formed by substituting any one or more alkylene groups in —(CH₂)h- with a group A; h is 0 to 15; the group A is selected from the group consisting of —O—, —S—, —C(O)—, —C(O)O—, —C(O)NH—, —CH(R^{C})—, —C(R′)(R″)—, —NH—, —N(R^{N})—, —S—S—, —C(R′)═C(R″)—, —C═C—, 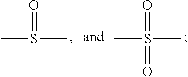
R^{C}, R^{N}, R′, and R″ in the group A represent that any one or more hydrogen atoms on a designated atom are substituted by a group B on condition that a normal valence of the designated atom is not exceeded and a stable compound is generated by substitution, and the designated atom is selected from the group consisting of a carbon atom and a nitrogen atom; the group B is selected from the group consisting of C1-C6 alkyl, C2-C6 alkenyl, C2-C6 alkynyl, cyano, hydroxyl, oxo, carboxyl, cycloalkyl, cycloalkenyl, heterocyclyl, heteroaryl, aryl, keto, alkoxycarbonyl, aryloxycarbonyl, heteroaryloxycarbonyl, and a halogen; the halogen is selected from the group consisting of F, Cl, Br, and I; and (O) in the group A represents a carbonyl oxygen atom;
in Formula 15 to Formula 20, Y and Z are independently absent, or selected from the group consisting of O, S, C(O), NH, CH₂, C(O)NH, NHC(O), C(O) O, OC(O), OC(O) O, OC(O)NH, NHC(O) O, and 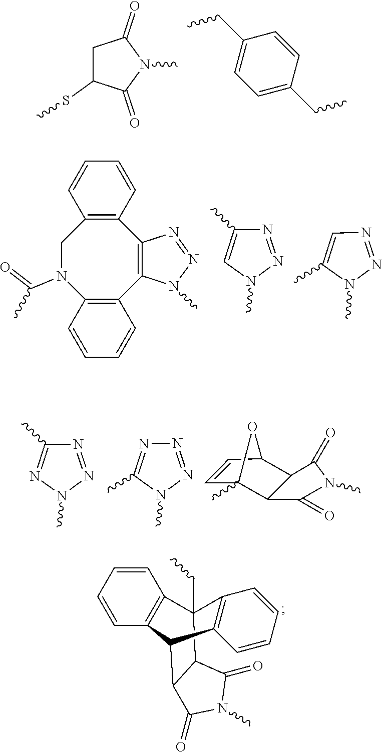
(O) in the groups Y and Z represents a carbonyl oxygen atom; and [symbol] represents a ligation site; and wherein
in Formula 15 to Formula 20, G represents an immunomodulatory inhibitor; n is 1 to 15; m and i are independently 0 to 5, and k and j are independently 1 to 20; R represents H; and B represents a nucleic acid base.

14. A method for treating an inflammation-related disease, comprising administering the small molecule drug-oligonucleotide conjugate to a subject in need thereof according to claim 3.

15. A method for treating an inflammation-related disease, comprising administering the small molecule drug-oligonucleotide conjugate to a subject in need thereof according to claim 4.

16. A method for treating an inflammation-related disease, comprising administering the small molecule drug-oligonucleotide conjugate to a subject in need thereof according to claim 5.

17. A method for treating an inflammation-related disease, comprising administering the small molecule drug-oligonucleotide conjugate to a subject in need thereof according to claim 6.

18. A method for treating an inflammation-related disease, comprising administering the small molecule drug-oligonucleotide conjugate to a subject in need thereof according to claim 7.

19. A method for treating an inflammation-related disease, comprising administering the small molecule drug-oligonucleotide conjugate to a subject in need thereof according to claim 8.

20. A method for treating an inflammation-related disease, comprising administering the small molecule drug-oligonucleotide conjugate to a subject in need thereof according to claim 12.

21. A method for treating an inflammation-related disease, comprising administering the small molecule drug-oligonucleotide conjugate to a subject in need thereof according to claim 13.


SEQUENCE LISTING (70 sequences)

SEQ ID NO: 1 - DNA, 19 bp, synthetic construct
  source 1..19: mol_type = other DNA
atcagacaacgaatcgggc

SEQ ID NO: 2 - DNA, 20 bp, synthetic construct
  source 1..20: mol_type = other DNA
aatactggtacattgacgcc

SEQ ID NO: 3 - DNA, 22 bp, synthetic construct
  source 1..22: mol_type = other DNA
  modified_base 20: mod_base = OTHER; note = n is t base modified with DBCO moiety
atcagacaacgaatcgggcntt

SEQ ID NO: 4 - DNA, 23 bp, synthetic construct
  source 1..23: mol_type = other DNA
  modified_base 21: mod_base = OTHER; note = n is t base modified with DBCO moiety
aatactggtacattgacgccntt

SEQ ID NO: 5 - DNA, 24 bp, synthetic construct
  source 1..24: mol_type = other DNA
  modified_base 20: mod_base = OTHER; note = n is t base modified with DBCO moiety
  modified_base 22: mod_base = OTHER; note = n is t base modified with DBCO moiety
atcagacaacgaatcgggcntntt

SEQ ID NO: 6 - DNA, 25 bp, synthetic construct
  source 1..25: mol_type = other DNA
  modified_base 21: mod_base = OTHER; note = n is t base modified with DBCO moiety
  modified_base 23: mod_base = OTHER; note = n is t base modified with DBCO moiety
aatactggtacattgacgccntntt

SEQ ID NO: 7 - DNA, 19 bp, synthetic construct
  source 1..19: mol_type = other DNA
atcagacaacgaatcgggc

SEQ ID NO: 8 - DNA, 20 bp, synthetic construct
  source 1..20: mol_type = other DNA
aatactggtacattgacgcc

SEQ ID NO: 9 - RNA, 21 nt, synthetic construct
  source 1..21: mol_type = other RNA
gcccgattcgttgtctgattt

SEQ ID NO: 10 - RNA, 21 nt, synthetic construct
  source 1..21: mol_type = other RNA
atcagacaacgaatcgggctt

SEQ ID NO: 11 - RNA, 24 nt, synthetic construct
  source 1..24: mol_type = other RNA
  modified_base 22: mod_base = OTHER; note = n is t base modified with DBCO moiety
gcccgattcgttgtctgatttntt

SEQ ID NO: 12 - RNA, 21 nt, synthetic construct
  source 1..21: mol_type = other RNA
atcagacaacgaatcgggctt

SEQ ID NO: 13 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
gcccgattcgttgtctgatttntntt

SEQ ID NO: 14 - RNA, 21 nt, synthetic construct
  source 1..21: mol_type = other RNA
atcagacaacgaatcgggctt

SEQ ID NO: 15 - RNA, 21 nt, synthetic construct
  source 1..21: mol_type = other RNA
gcgtcaatgtaccagtatttt

SEQ ID NO: 16 - RNA, 21 nt, synthetic construct
  source 1..21: mol_type = other RNA
aatactggtacattgacgcct

SEQ ID NO: 17 - RNA, 24 nt, synthetic construct
  source 1..24: mol_type = other RNA
  modified_base 22: mod_base = OTHER; note = n is t base modified with DBCO moiety
gcgtcaatgtaccagtattttntt

SEQ ID NO: 18 - RNA, 21 nt, synthetic construct
  source 1..21: mol_type = other RNA
aatactggtacattgacgcct

SEQ ID NO: 19 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
gcgtcaatgtaccagtattttntntt

SEQ ID NO: 20 - RNA, 21 nt, synthetic construct
  source 1..21: mol_type = other RNA
aatactggtacattgacgcct

SEQ ID NO: 21 - DNA, 45 bp, synthetic construct
  source 1..45: mol_type = other DNA
atcagacaacgaatcgggctttttttttttttttttttttttttt

SEQ ID NO: 22 - DNA, 46 bp, synthetic construct
  source 1..46: mol_type = other DNA
aatactggtacattgacgcctttttttttttttttttttttttttt

SEQ ID NO: 23 - RNA, 27 nt, synthetic construct
  source 1..27: mol_type = other RNA
  modified_base 23: mod_base = OTHER; note = n is t base modified with DBCO moiety
  modified_base 25: mod_base = OTHER; note = n is t base modified with DBCO moiety
ctctaatgagtttagtccgattntntt

SEQ ID NO: 24 - RNA, 22 nt, synthetic construct
  source 1..22: mol_type = other RNA
tcggactaaactcattagagtt

SEQ ID NO: 25 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
aggctgatctgttgccgtattntntt

SEQ ID NO: 26 - RNA, 21 nt, synthetic construct
  source 1..21: mol_type = other RNA
tacggcaacagatcagccttg

SEQ ID NO: 27 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
cagcaaccctgagtccctattntntt

SEQ ID NO: 28 - RNA, 21 nt, synthetic construct
  source 1..21: mol_type = other RNA
tagggactcagggttgctgtt

SEQ ID NO: 29 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
gacaaccaactagtggtgcttntntt

SEQ ID NO: 30 - RNA, 21 nt, synthetic construct
  source 1..21: mol_type = other RNA
gcaccactagttggttgtctt

SEQ ID NO: 31 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
aagagatcctggtcctgaattntntt

SEQ ID NO: 32 - RNA, 21 nt, synthetic construct
  source 1..21: mol_type = other RNA
ttcaggaccaggatctcttgc

SEQ ID NO: 33 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
ggaaggcagtgtcactcatttntntt

SEQ ID NO: 34 - RNA, 21 nt, synthetic construct
  source 1..21: mol_type = other RNA
atgagtgacactgccttcctt

SEQ ID NO: 35 - RNA, 32 nt, synthetic construct
  source 1..32: mol_type = other RNA
  modified_base 28: mod_base = OTHER; note = n is t base modified with DBCO moiety
  modified_base 30: mod_base = OTHER; note = n is t base modified with DBCO moiety
acaaccatcaccacactggatacggttntntt

SEQ ID NO: 36 - RNA, 27 nt, synthetic construct
  source 1..27: mol_type = other RNA
ccgtatccagtgtggtgatggttgttt

SEQ ID NO: 37 - RNA, 29 nt, synthetic construct
  source 1..29: mol_type = other RNA
  modified_base 25: mod_base = OTHER; note = n is t base modified with DBCO moiety
  modified_base 27: mod_base = OTHER; note = n is t base modified with DBCO moiety
gtctcagcctcttctcattcctttntntt

SEQ ID NO: 38 - RNA, 24 nt, synthetic construct
  source 1..24: mol_type = other RNA
aggaatgagaagaggctgagactt

SEQ ID NO: 39 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
gtggacttgaagaaatttattntntt

SEQ ID NO: 40 - RNA, 21 nt, synthetic construct
  source 1..21: mol_type = other RNA
taaatttcttcaagtccactt

SEQ ID NO: 41 - RNA, 28 nt, synthetic construct
  source 1..28: mol_type = other RNA
  modified_base 24: mod_base = OTHER; note = n is t base modified with DBCO moiety
  modified_base 26: mod_base = OTHER; note = n is t base modified with DBCO moiety
ggattacaaggatgacgaaggttntntt

SEQ ID NO: 42 - RNA, 23 nt, synthetic construct
  source 1..23: mol_type = other RNA
ttcgtcatccttgtaatccattt

SEQ ID NO: 43 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
gagtcggtctggaaatcaattntntt

SEQ ID NO: 44 - RNA, 21 nt, synthetic construct
  source 1..21: mol_type = other RNA
ttgatttccagaccgactctt

SEQ ID NO: 45 - RNA, 26 nt, synthetic construct
  source 1..26: mol_type = other RNA
  modified_base 22: mod_base = OTHER; note = n is t base modified with DBCO moiety
  modified_base 24: mod_base = OTHER; note = n is t base modified with DBCO moiety
ggtgaaatgtacttaaatcttntntt

SEQ ID NO: 46 - RNA, 21 nt, synthetic construct
  source 1..21: mol_type = other RNA
gatttaagtacatttcacctt

SEQ ID NO: 47 - RNA, 28 nt, synthetic construct
  source 1..28: mol_type = other RNA
  modified_base 24: mod_base = OTHER; note = n is t base modified with DBCO moiety
  modified_base 26: mod_base = OTHER; note = n is t base modified with DBCO moiety
ctgtatggcgacattctccaattntntt

SEQ ID NO: 48 - RNA, 23 nt, synthetic construct
  source 1..23: mol_type = other RNA
ttggagaatgtcgccatacagtt

SEQ ID NO: 49 - RNA, 28 nt, synthetic construct
  source 1..28: mol_type = other RNA
  modified_base 24: mod_base = OTHER; note = n is t base modified with DBCO moiety
  modified_base 26: mod_base = OTHER; note = n is t base modified with DBCO moiety
atgagcgtgtagagaggacaattntntt

SEQ ID NO: 50 - RNA, 23 nt, synthetic construct
  source 1..23: mol_type = other RNA
ttgtcctctctacacgctcattt

SEQ ID NO: 51 - DNA, 27 bp, synthetic construct
  source 1..27: mol_type = other DNA
  modified_base 23: mod_base = OTHER; note = n is t base modified with DBCO moiety
  modified_base 25: mod_base = OTHER; note = n is t base modified with DBCO moiety
tcggactaaactcattagagttntntt

SEQ ID NO: 52 - DNA, 27 bp, synthetic construct
  source 1..27: mol_type = other DNA
  modified_base 23: mod_base = OTHER; note = n is t base modified with DBCO moiety
  modified_base 25: mod_base = OTHER; note = n is t base modified with DBCO moiety
ggtacttctgccatggctgcttntntt

SEQ ID NO: 53 - DNA, 32 bp, synthetic construct
  source 1..32: mol_type = other DNA
  modified_base 28: mod_base = OTHER; note = n is t base modified with DBCO moiety
  modified_base 30: mod_base = OTHER; note = n is t base modified with DBCO moiety
cattacagctctgctccccagcatcttntntt

SEQ ID NO: 54 - DNA, 23 bp, synthetic construct
  source 1..23: mol_type = other DNA
  modified_base 19: mod_base = OTHER; note = n is t base modified with DBCO moiety
  modified_base 21: mod_base = OTHER; note = n is t base modified with DBCO moiety
cagtgctcatggtgtcttntntt

SEQ ID NO: 55 - DNA, 28 bp, synthetic construct
  source 1..28: mol_type = other DNA
  modified_base 24: mod_base = OTHER; note = n is t base modified with DBCO moiety
  modified_base 26: mod_base = OTHER; note = n is t base modified with DBCO moiety
ttcaggaccaggatctcttgcttntntt

SEQ ID NO: 56 - DNA, 26 bp, synthetic construct
  source 1..26: mol_type = other DNA
  modified_base 22: mod_base = OTHER; note = n is t base modified with DBCO moiety
  modified_base 24: mod_base = OTHER; note = n is t base modified with DBCO moiety
atgagtgacactgccttccttntntt

SEQ ID NO: 57 - DNA, 32 bp, synthetic construct
  source 1..32: mol_type = other DNA
  modified_base 28: mod_base = OTHER; note = n is t base modified with DBCO moiety
  modified_base 30: mod_base = OTHER; note = n is t base modified with DBCO moiety
ccgtatccagtgtggtgatggttgtttntntt

SEQ ID NO: 58 - DNA, 27 bp, synthetic construct
  source 1..27: mol_type = other DNA
  modified_base 23: mod_base = OTHER; note = n is t base modified with DBCO moiety
  modified_base 25: mod_base = OTHER; note = n is t base modified with DBCO moiety
aacccatcggctggcaccacttntntt

SEQ ID NO: 59 - DNA, 48 bp, synthetic construct
  source 1..48: mol_type = other DNA
agctgctgccccgacccaaacctttttttttttttttttttttttttt

SEQ ID NO: 60 - DNA, 47 bp, synthetic construct
  source 1..47: mol_type = other DNA
ttcgtcatccttgtaatccattttttttttttttttttttttttttt

SEQ ID NO: 61 - DNA, 43 bp, synthetic construct
  source 1..43: mol_type = other DNA
ttgatttccagaccgactttttttttttttttttttttttttt

SEQ ID NO: 62 - DNA, 45 bp, synthetic construct
  source 1..45: mol_type = other DNA
gatttaagtacatttcacctttttttttttttttttttttttttt

SEQ ID NO: 63 - DNA, 47 bp, synthetic construct
  source 1..47: mol_type = other DNA
ttggagaatgtcgccatacagtttttttttttttttttttttttttt

SEQ ID NO: 64 - DNA, 46 bp, synthetic construct
  source 1..46: mol_type = other DNA
ttgtcctctctacacgctcatttttttttttttttttttttttttt

SEQ ID NO: 65 - DNA, 33 bp, synthetic construct
  source 1..33: mol_type = other DNA
atcagacaacgaatcgggctttttttttttttt

SEQ ID NO: 66 - DNA, 34 bp, synthetic construct
  source 1..34: mol_type = other DNA
aatactggtacattgacgcctttttttttttttt

SEQ ID NO: 67 - DNA, 33 bp, synthetic construct; identical to SEQ ID NO: 65
  source 1..33: mol_type = other DNA

SEQ ID NO: 68 - DNA, 34 bp, synthetic construct; identical to SEQ ID NO: 66
  source 1..34: mol_type = other DNA

SEQ ID NO: 69 - DNA, 33 bp, synthetic construct; identical to SEQ ID NO: 65
  source 1..33: mol_type = other DNA

SEQ ID NO: 70 - DNA, 34 bp, synthetic construct; identical to SEQ ID NO: 66
  source 1..34: mol_type = other DNA